(19) United States
(12) Patent Application Publication
(10) Pub. No.: US 2019/0214577 A1
(43) Pub. Date: Jul. 11, 2019

(54) THERMALLY ACTIVATED DELAYED FLUORESCENCE MATERIAL, POLYMER, MIXTURE, FORMULATION, AND ORGANIC ELECTRONIC DEVICE

(71) Applicant: GUANGZHOU CHINARAY OPTOELECTRONIC MATERIALS LTD., Guangzhou, Guangdong (CN)

(72) Inventors: Junyou PAN, Guangzhou, Guangdong (CN); Hong HUANG, Guangzhou, Guangdong (CN)

(21) Appl. No.: 15/781,205

(22) PCT Filed: Sep. 28, 2016
(86) PCT No.: PCT/CN2016/100562

§ 371 Date: Jun. 4, 2018

(30) Foreign Application Priority Data
Dec. 4, 2015 (CN) 201510888883.9

(51) Int. Cl. H01L 51/00; C09K 11/06; C07D 209/86; C08G 61/12; C07D 403/14; C07F 5/02

(52) U.S. Cl. CPC H01L 51/0072; C09K 11/06; C07D 209/86; H01L 51/5016; C07D 403/14; H01L 51/0067; C07F 5/027; C08G 61/124

(57) ABSTRACT

Disclosed is a thermally activated delayed fluorescence material capable of improving luminous efficiency and stability, a polymer, a mixture, a formulation, and an organic electronic device comprising the same. This thermally activated delayed fluorescence material comprises a conjugated unit comprising at least two aromatic rings or heteroaromatic rings, so as to achieve thermally activated delayed fluorescence (TADF) characteristics. This thermally activated delayed fluorescence material can be used as a TADF luminescent material to improve the luminous efficiency and stability of the electroluminescent device containing such a thermally activated delayed fluorescence material by cooperating with a suitable host material, thereby providing a solution of the luminescent device which has low manufacturing cost, a high efficiency, a long life, and being low in roll-off.

### TECHNICAL FIELD

The present disclosure relates to the field of organic opto-electronic materials, especially to a thermally activated delayed fluorescence material, and a polymer, a mixture, a formulation, and an organic electronic device comprising the same.

### BACKGROUND

Organic light emitting diode (OLED) has great application potential in opto-electronic device (such as flat-panel display and lighting device) because of the diversity in synthesis, relative low manufacturing cost, and excellent opto-electronic properties of organic semiconductor materials.

Various luminescent materials based on fluorescence and phosphorescence have been developed to improve the luminous efficiency of the OLED. The OLED using a fluorescent material has a high reliability. However, since the single to triplet ratio of excitons is 1:3 under electrical excitation, its internal quantum efficiency for electroluminescence is limited to 25%. In contrast, the internal quantum efficiency for electroluminescence of the OLED using a phosphorescent material has reached almost 100%. However, the roll-off effect of the phosphorescent OLED, i.e., the luminous efficiency is rapidly decreased with an increase of current or luminance, is a significant issue which is particularly detrimental to applications at high luminance.

So far, practicable and useful phosphorescent materials are iridium and platinum complexes, whose costs are quite high since the raw materials are rare and expensive, and the syntheses of the complexes are rather complicated. In order to solve this problem, Adachi proposed the concept of reverse intersystem crossing so that an organic compound can be used to substitute the metal complexes to achieve the efficiency comparable to that of the phosphorescent OLED. Such concept has been realized in various combinations of materials, such as 1) by using an exciplex, see Adachi et al., Nature Photonics, Vol 6, p 253 (2012); and 2) by using a thermally activated delayed fluorescent (TADF) material, see Adachi et al., Nature Vol 492, 234 (2012). So far, a series of red and green TADF materials with a high luminous efficiency has been developed. However, the blue TADF material is rare, and the properties, especially the lifetime of the OLED device of the TADF material are far from enough for practical application. Furthermore, the donor groups and the acceptor groups are linked to each other in most organic compounds with TADF properties, leading a complete separation between the electron cloud distribution of the highest occupied molecular orbital (HOMO) and the electron cloud distribution of the lowest occupied molecular orbital (LOMO), and thus reducing the energy difference, ΔE(S₁−T₁), between the singlet state (S₁) and trilet state (T₁) of the organic compound, which is adverse to the luminous efficiency and the stability.

### SUMMARY

In view of this, it is necessary to provide a thermally activated delayed fluorescence material capable of improving the luminous efficiency and the stability, and a polymer, a mixture, a formulation, and an organic electronic device comprising the same.

A thermally activated delayed fluorescence material comprises a structure unit represented by the following general formula (1):

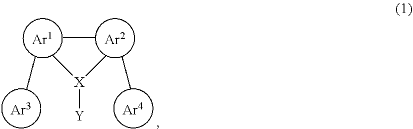

wherein Ar¹, Ar², Ar³, or Ar⁴ is selected from aromatic ring containing 3 to 20 carbon atoms, R₁ substituted aromatic ring containing 3 to 20 carbon atoms, heteroaromatic ring containing 2 to 20 carbon atoms, R₁ substituted heteroaromatic ring containing 2 to 20 carbon atoms, non-aromatic ring containing 1 to 20 carbon atoms, or R₁ substituted non-aromatic ring containing 1 to 20 carbon atoms;

X is a doubly bridging group or a triply bridging group linked to Ar¹, Ar², or Y by a single bond or a double bond;

Y is linked to X by the single bond or the double bond;

Y is selected from nothing, aromatic ring containing 3 to 20 carbon atoms, heteroaromatic ring containing 2 to 20 carbon atoms, non-aromatic ring containing 1 to 20 carbon atoms, B(OR₂)₂, Si(R₂)₃, alkyl ether group, alkyl thioether group containing 1 to 10 carbon atoms, alkyl, alkoxy, alkenyl, alkynyl, alkyl ether group containing 3 to 10 carbon atoms, deuterides thereof, fluorides thereof, R₁ substituted groups thereof, and a combination of two, three, or four thereof;

R₁ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, cycloalkyl, or alkyl ether group containing 3 to 10 carbon atoms;

R₂ is selected from H, D, aliphatic alkyl containing 1 to 10 carbon atoms, aromatic hydrocarbon group containing 1 to 10 carbon atoms, aromatic ring containing 5 to 10 ring atoms, substituted aromatic ring containing 5 to 10 ring atoms, heteroaryl containing 5 to 10 ring atoms, or substituted heteroaryl containing 5 to 10 ring atoms.

A polymer comprises one repeating unit containing the thermally activated delayed fluorescence material described above.

A mixture comprises the thermally activated delayed fluorescence material described above or the polymer described above.

The mixture further comprises an organic functional material selected from at least one of a hole injection material, a hole transport material, an electron injection material, an electron transport material, a hole blocking material, an electron blocking material, a luminescent material, a host material, and an organic dye.

A formulation comprises the thermally activated delayed fluorescence material described above or the polymer described above.

The formulation further comprises an organic solvent.

An organic electronic device comprises the thermally activated delayed fluorescence material described above, the polymer described above, or the mixture described above.

The thermally activated delayed fluorescence material comprises a conjugated unit having at least two aromatic rings or heteroaromatic rings, so as to achieve the thermally activated delayed fluorescence (TADF) property. The thermally activated delayed fluorescence material can be used as a TADF luminescent material to improve the luminous efficiency and the stability of the electroluminescent device containing the thermally activated delayed fluorescence material by cooperating with a suitable host material, thereby providing a technical solution of the electroluminescent device with a low manufacturing cost, a high efficiency, a long lifetime, and a low roll-off effect.

### BRIEF DESCRIPTION OF THE DRAWINGS

FIG. 1 shows a HOMO orbital distribution of the compound obtained in Example 1.

FIG. 2 shows a LOMO orbital distribution of the compound obtained in Example 1.

FIG. 3 shows the HOMO orbital distribution of the compound obtained in Example 3.

FIG. 4 shows the LOMO orbital distribution of the compound obtained in Example 3.

### DETAILED DESCRIPTION

In order to make the objects, technical solutions and advantages of the present disclosure to be understood more clearly, the present disclosure will be described in further details with the accompanying drawings and the following embodiments. Although the present disclosure is disclosed hereinafter with reference to preferred embodiments in detail, it also can be implemented in other different embodiments and those skilled in the art may modify and vary the embodiments without departing from the spirit and scope of the present disclosure. Therefore, the present disclosure should not be limited by the embodiments disclosed herein.

In the present disclosure, the terms “formulation” and “printing ink” (or “ink”) have the same meaning and are interchangeable in use.

In the present disclosure, the terms “host”, “matrix”, “host material” and “matrix material” have the same meaning and are interchangeable in use.

In the present disclosure, the terms “metal organic complex” and “metal organic coordination complex” and “organometallic complex” have the same meaning and are interchangeable in use.

A thermally activated delayed fluorescence material includes a structure unit represented by the following general formula (1):

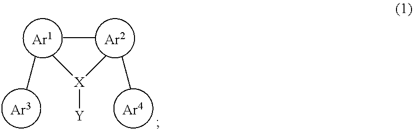

wherein Ar¹, Ar², Ar³, or Ar⁴ is selected from aromatic ring containing 3 to 20 carbon atoms, R₁ substituted aromatic ring containing 3 to 20 carbon atoms, heteroaromatic ring containing 2 to 20 carbon atoms, R₁ substituted heteroaromatic ring containing 2 to 20 carbon atoms, non-aromatic ring containing 1 to 20 carbon atoms, or R₁ substituted non-aromatic ring containing 1 to 20 carbon atoms;

X is a doubly bridging group or a triply bridging group linked to Ar¹, Ar², or Y by a single bond or a double bond;

Y is connected to X by a single bond or a double bond;

Y is selected from nothing, aromatic ring containing 3 to 20 carbon atoms, heteroaromatic ring containing 2 to 20 carbon atoms, non-aromatic ring containing 1 to 20 carbon atoms, B(OR₂)₂, Si(R₂)₃, alkyl ether group, alkyl thioether group containing 1 to 10 carbon atoms, alkyl (straight chain alkyl, branched chain alkyl, cycloalkyl), alkoxy, alkenyl, alkynyl, alkyl ether group containing 3 to 10 carbon atoms, deuterides thereof, fluorides thereof, R₁ substituted groups thereof, and a combination of two, three, or four thereof;

R₁ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, cycloalkyl, or alkyl ether group containing 3 to 10 carbon atoms;

R₂ is selected from H, D, aliphatic alkyl containing 1 to 10 carbon atoms, aromatic hydrocarbon group containing 1 to 10 carbon atoms, aromatic ring containing 5 to 10 ring atoms, substituted aromatic ring containing 5 to 10 ring atoms, heteroaryl containing 5 to 10 ring atoms, or substituted heteroaryl containing 5 to 10 ring atoms.

In one embodiment, the thermally activated delayed fluorescence material satisfies: ΔE(S₁−T₁)≤0.30 eV.

In one embodiment, X is selected from one of the following groups:

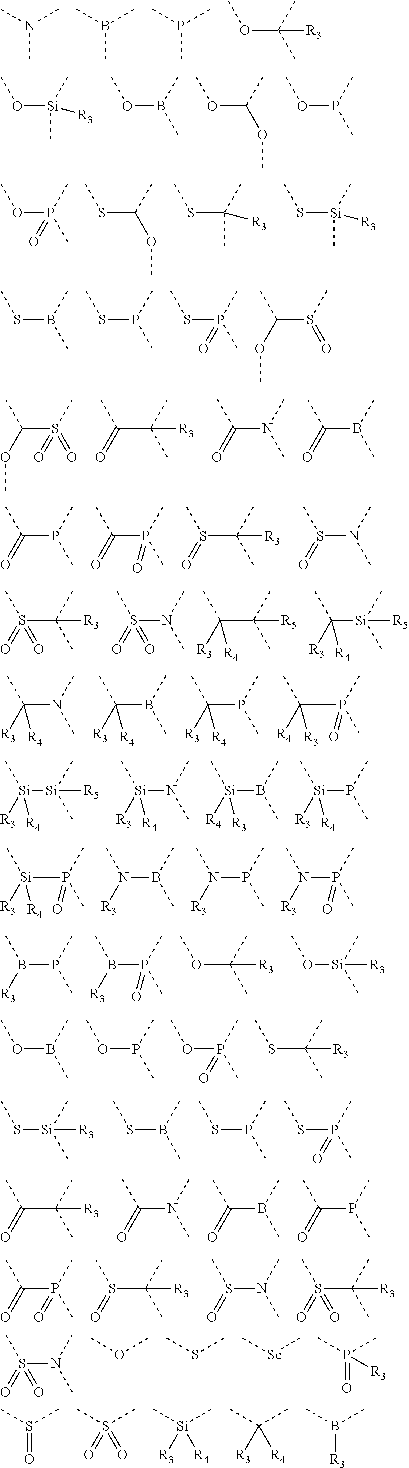

wherein R₃, R₄, or R₅ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, cycloalkyl, or alkyl ether group containing 3 to 10 carbon atoms;

the dashed line bonds in the above groups represent bonds linked to Ar¹, Ar², and Y.

In one embodiment, X is selected from one of the following groups:

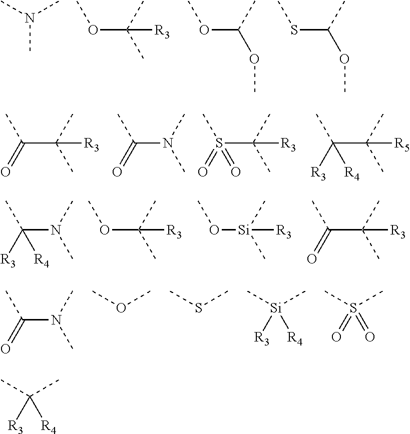

wherein R₃, R₄, or R₅ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, cycloalkyl, or alkyl ether group containing 3 to 10 carbon atoms;

the dashed line bonds in the above groups represent bonds linked to Ar¹, Ar², and Y.

In one embodiment, X is selected from one of the following groups:

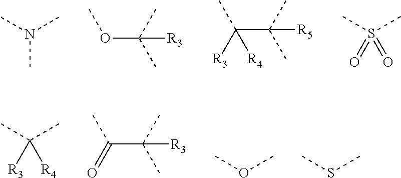

wherein R₃, R₄, or R₅ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, cycloalkyl, or alkyl ether group containing 3 to 10 carbon atoms;

the dashed line bonds in the above groups represent bonds linked to Ar¹, Ar², and Y. In one embodiment, the thermally activated delayed fluorescence material includes a structure unit represented by the following general formula (2):

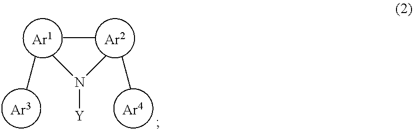

wherein all symbols in the general formula (2) is the same as that defined in the general formula (1).

In one embodiment, Ar¹, Ar², Ar³, or Ar⁴ is selected from aromatic ring containing 3 to 20 carbon atoms, R¹ substituted aromatic ring containing 3 to 20 carbon atoms, heteroaromatic ring containing 2 to 20 carbon atoms, or R¹ substituted heteroaromatic ring containing 2 to 20 carbon atoms.

In one embodiment, the aromatic ring contains 5 to 18 carbon atoms in its ring system.

In one embodiment, the heteroaromatic ring contains 2 to 18 carbon atoms and at least one heteroatom in its ring system, and a total number of the carbon atoms and the at least one heteroatom is at least 4.

In another embodiment, the aromatic ring contains 5 to 16 carbon atoms in its ring system.

In another embodiment, the heteroaromatic ring contains 2 to 16 carbon atoms and at least one heteroatom in its ring system.

In another embodiment, the aromatic ring contains 5 to 13 carbon atoms in its ring system.

In another embodiment, the heteroaromatic ring contains 2 to 13 carbon atoms and at least one heteroatom in its ring system.

In one embodiment, the heteroatom is selected from at least one of Si, N, P, O, S, and Ge.

In another embodiment, the heteroatom is selected from at least one of Si, N, P, O, and S.

In the present disclosure, the aromatic ring (aromatic group, aryl) refers to a hydrocarbyl including at least one aromatic cyclic structure such as a single ring group and a ring system with multiple rings. The heteroaromatic ring (heteroaromatic group, heteroaryl) refers to a hydrocarbyl including at least one aromatic heterocyclic structure (which includes a heteroatom) such as a single ring group and a ring system with multiple rings. The ring system with multiple rings may include two or more rings in which two carbon atoms are shared by two adjacent rings, that is the two or more rings form a condensed ring. At least one ring of the ring system with multiple rings belongs to aromatics or heteroaromatics. For the purpose of the present disclosure, the aromatic ring or the heteroaromatic ring may include an aryl system or a heteroaryl system in which a plurality of aryls or heteroaryls may be interrupted by a short non-aromatic unit. Therefore, for the purpose of the present disclosure, some systems such as 9,9′-spirobifluorene, 9,9′-diarylfluorene, triarylamine, and diaryl ether are also considered to be the aromatic rings.

In one embodiment, examples of the aromatic group include benzene, naphthalene, anthracene, phenanthrene, perylene, tetracene, pyrene, benzopyrene, triphenylene, acenaphthene, fluorene, and derivatives thereof.

In one embodiment, examples of the heteroaromatic group include furan, benzofuran, thiophene, benzothiophene, pyrrole, pyrazole, triazole, imidazole, oxazole, oxadiazole, thiazole, tetrazole, indole, carbazole, pyrroloimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, cinnoline, quinoxaline, phenanthridine, perimidine, quinazoline, quinazolinone, and derivatives thereof.

In one embodiment, at least one of Ar¹, Ar², Ar³, and Ar⁴ includes non-aromatic ring containing 1 to 20 carbon atoms or R₁ substituted non-aromatic ring containing 1 to 20 carbon atoms.

In one embodiment, the non-aromatic ring includes 1 to 10 (such as 1 to 6) carbon atoms in its ring system. The non-aromatic ring includes not only saturated ring system, but also partially unsaturated ring system, which can be unsubstituted or substituted with one or more R₁ groups. R₁ groups may be the same or different in multiple occurrences, and may further includes one or more heteroatoms which are selected from Si, N, P, O, S, and/or Ge in one embodiment, and are selected from Si, N, P, O, and/or S in another embodiment. The non-aromatic ring may be, for example, cyclohexyl-like system or piperidine-like system, and cyclooctadiene-like ring system. The term “non-aromatic ring” also includes condensed non-aromatic ring.

In the present disclosure, H atom in NH or bridging group CH₂ may be substituted with R¹ group, and R¹ may be selected from (1) C1˜C10 alkyl, such as methyl, ethyl, n-propyl, isopropyl, cyclopropyl, n-butyl, isobutyl, sec-butyl, tert-butyl, cyclobutyl, 2-methylbutyl, n-pentyl, n-hexyl, cyclohexyl, n-heptyl, cycloheptyl, n-octyl, cyclooctyl, 2-ethylhexyl, trifluoromethyl, pentafluoromethyl, 2,2,2-trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl and octynyl; (2) C1˜C10 alkoxy, such as methoxy, ethoxy, n-propoxy, isopropoxy, n-butoxy, isobutoxy, sec-butoxy, tert-butoxy or 2-methylbutoxy; (3) C2˜C10 aryl or heteroaryl, which may be univalent or bivalent according to use, in each case, may be substituted with the R¹ group as mentioned above, and may be linked to other aromatic rings or heteroaromatic rings on any desirable position, such as benzene, naphthalene, anthracene, pyrene, dihydropyrene, chrysene, perylene, fluoranthene, naphthacene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthaimidazole, phenanthroimidazole, imidazopyridine, imidazopyrazine, imidazoquinoxaline, oxazole, benzoxazole, naphthoxazole, anthraxoxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, 1,5-naphthyridine, nitrocarbazole, benzoporphyrin, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole. 1,2,4,5-tetrazine, 1,2,3,4-tetrazine, 1,2,3,5-tetrazine, purine, pteridine, indolizine, and benzothiadiazole. For the purpose of the present disclosure, except the aryl and heteroaryl as mentioned above, the aromatic and heteroaromatic rings also refer to biphenylene, triphenylene, fluorene, spirobifluorene, dihydrophenanthrene, tetrahydropyrene, and cis-indenofluorene or trans-indenofluorene.

In one embodiment, in the compound according to the general formula (1) or the general formula (2), Ar¹ or Ar² is selected from aromatic ring containing 3 to 10 carbon atoms, heteroaromatic ring containing 2 to 10 carbon atoms, or non-aromatic ring containing 2 to 10 carbon atoms, which can be unsubstituted or substituted with R₁. In one embodiment, the aromatic ring or the heteroaromatic ring is benzene, naphthalene, anthracene, phenanthrene, pyridine, pyrene or thiophene.

In one embodiment, Ar¹ or Ar² is selected from one of the following groups:

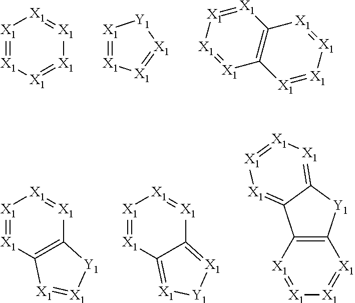

wherein X₁ is CR⁶ or N;

Y₁ is selected from CR⁷R⁸, SiR⁹R¹⁰, NR¹¹, C(═O) S, or O;

R₆, R₇, R₈, R₉, R₁₀, or R₁₁ is selected from H, D, straight chain alkyl containing 1 to 20 carbon atoms, alkoxy containing 1 to 20 carbon atoms, thioalkoxy containing 1 to 20 carbon atoms, branched chain alkyl containing 3 to 20 carbon atoms, cyclic alkyl containing 3 to 20 carbon atoms, alkoxy containing 3 to 20 carbon atoms, thioalkoxy containing 3 to 20 carbon atoms, silyl containing 3 to 20 carbon atoms, substituted ketone group containing 1 to 20 carbon atoms, alkoxycarbonyl containing 2 to 20 carbon atoms, aryloxycarbonyl containing 7 to 20 carbon atoms, cyano group, carbamoyl, haloformyl, formyl, isocyano group, isocyanate, thiocyanate, isothiocyanate, hydroxy, nitryl, CF₃, Cl, Br, F, crosslinkable group, aromatic ring containing 5 to 40 carbon atoms, substituted aromatic ring containing 5 to 40 carbon atoms, heteroaromatic ring containing 5 to 40 carbon atoms, substituted heteroaromatic ring containing 5 to 40 carbon atoms, aryloxy containing 5 to 40 carbon atoms, heteroaryloxy containing 5 to 40 carbon atoms, and combinations thereof;

wherein one or more of R₆, R₇, R₈, R₉, R₁₀, and R₁₁ may form a monocyclic or polycyclic aliphatic or aromatic ring between one another and/or with a ring linked to them.

In one embodiment, Ar¹ or Ar² is selected from the following groups and the following groups substituted with

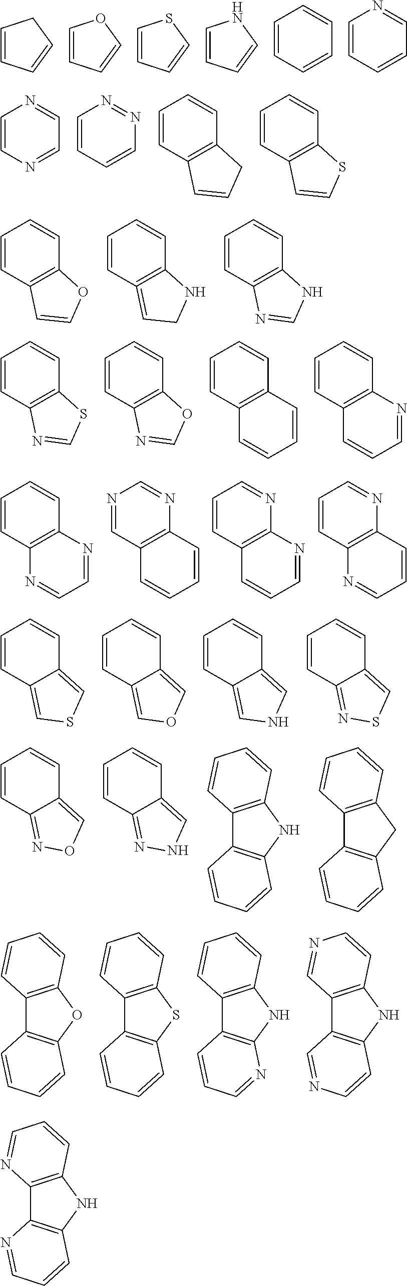

In one embodiment, the thermally activated delayed fluorescence material of the present disclosure has a relatively high triplet state energy level T₁ which is larger than or equal to 2.0 eV. In another embodiment, the triplet state energy level T₁ is larger than or equal to 2.2 eV. In another embodiment, the triplet state energy level T₁ is larger than or equal to 2.4 eV. In another embodiment, the triplet state energy level T₁ is larger than or equal to 2.6 eV. In another embodiment, the triplet state energy level T₁ is larger than or equal to 2.8 eV.

The triplet state energy level T₁ of the organic compound usually depends on its sub-structure with a largest conjugated system. In general, T₁ decreases with the conjugated system getting larger.

In one embodiment, a compound represented by the following general formula (1-a) has the largest conjugated system:

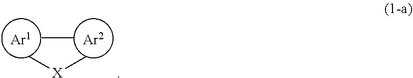

The number of the ring atoms of the compound represented by the general formula (1-a), excluding the substituent group, is not more than 30 in one embodiment, is not more than 26 in another embodiment, is not more than 22 in another embodiment, and is not more than 20 in another embodiment.

In one embodiment, the compound represented by the general formula (1) or the general formula (2) is selected from one of compounds represented by the following general formulas:

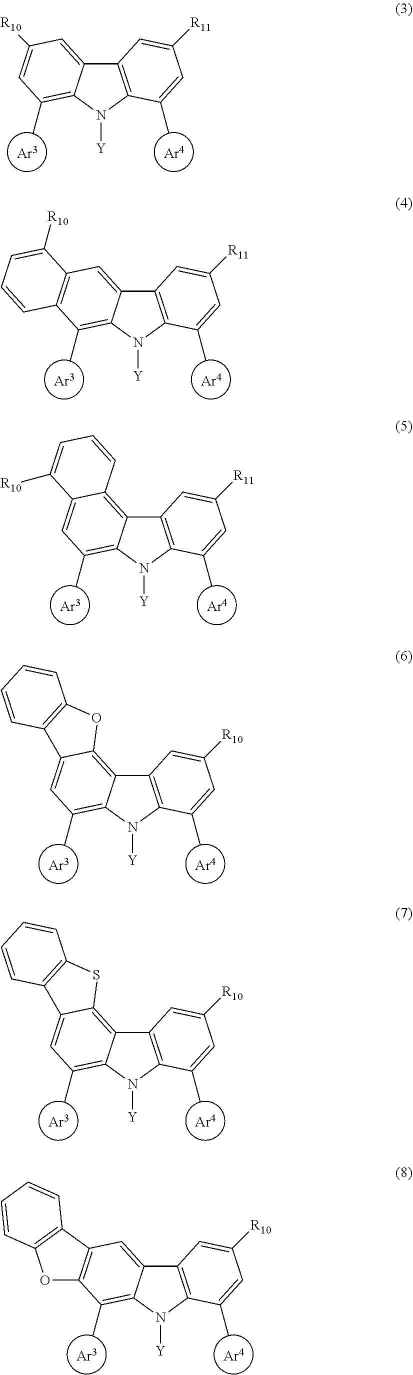

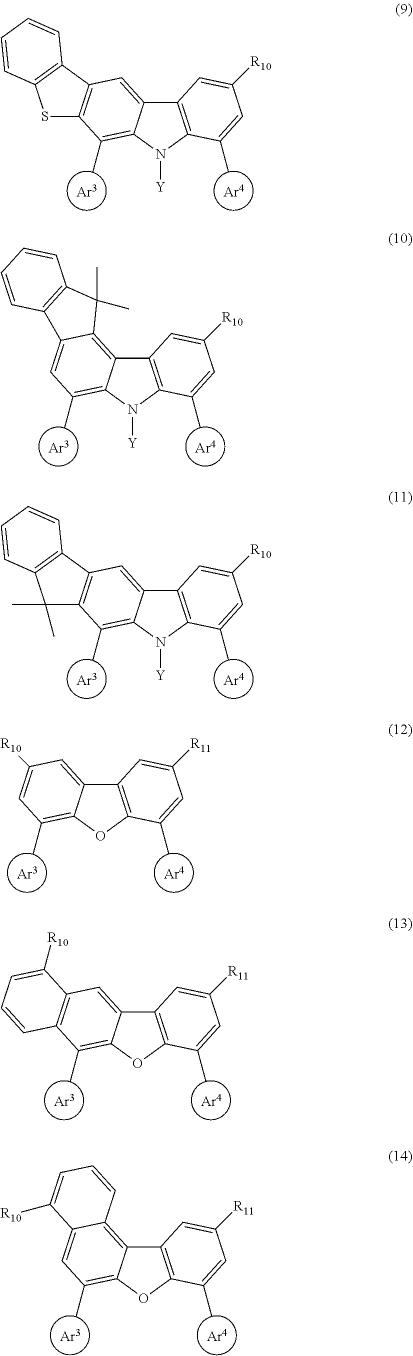

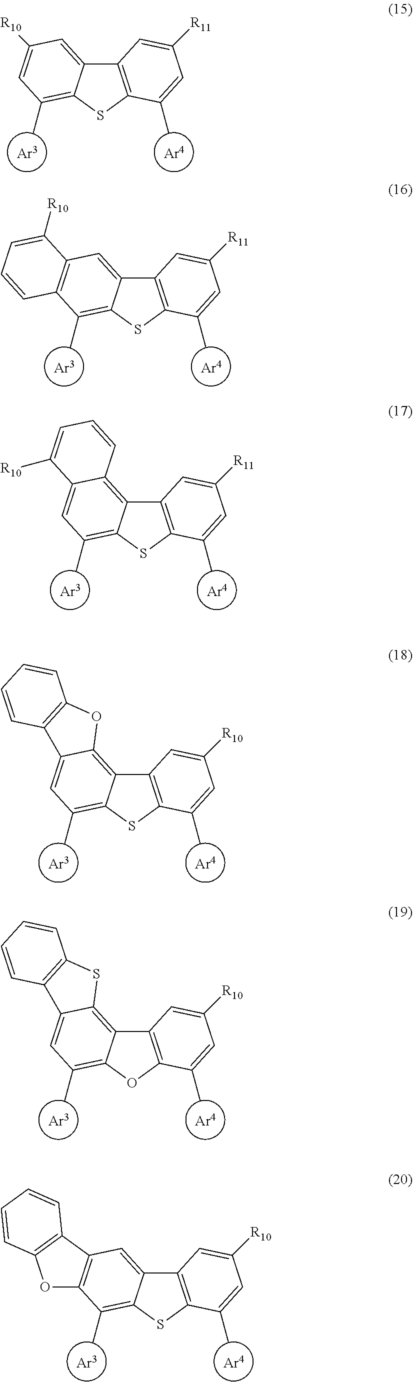

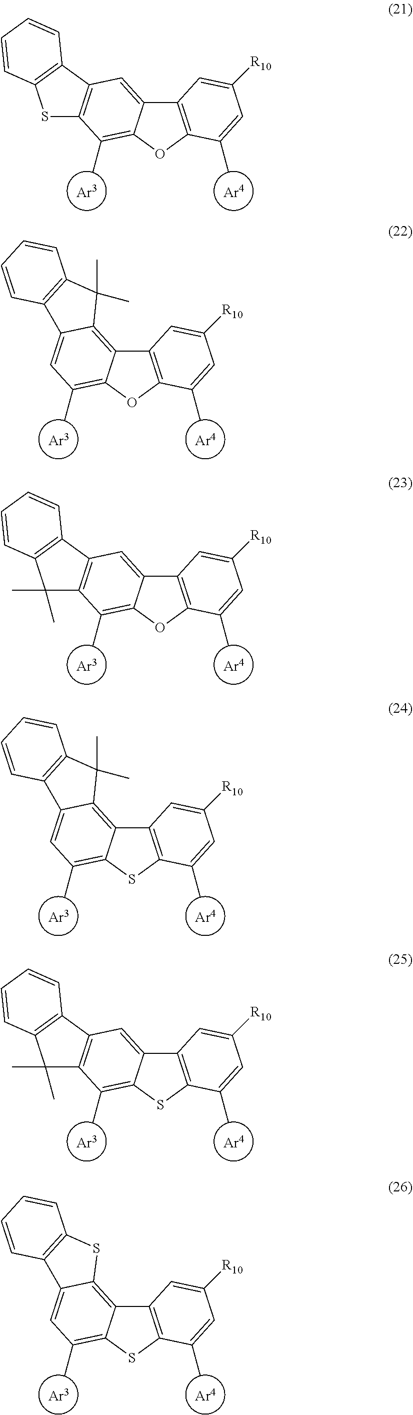

wherein R₁₀ and R₁₁ are defined in the same way as R₁.

In one embodiment, in the compound represented by the general formula (1) or the general formula (2), Ar³ or Ar⁴, in multiple occurrences, includes one or combinations of the following structure units and substituted following structure units:

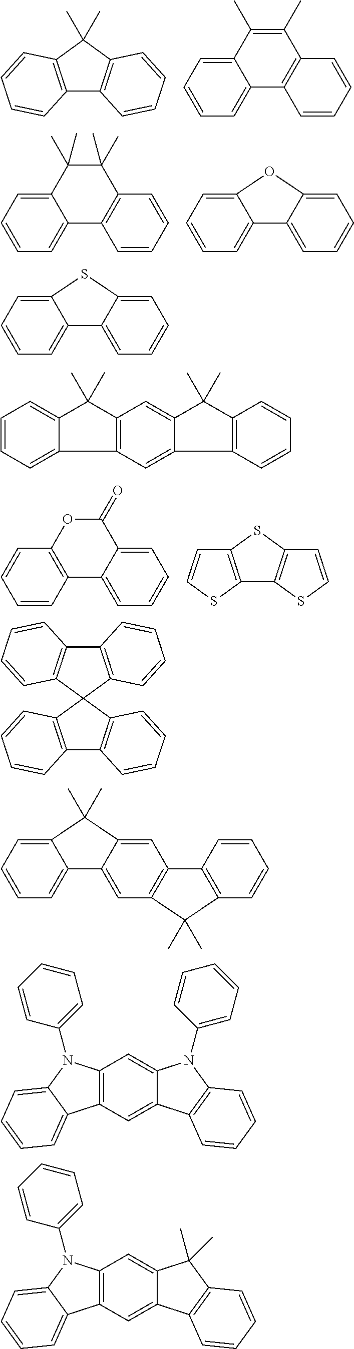

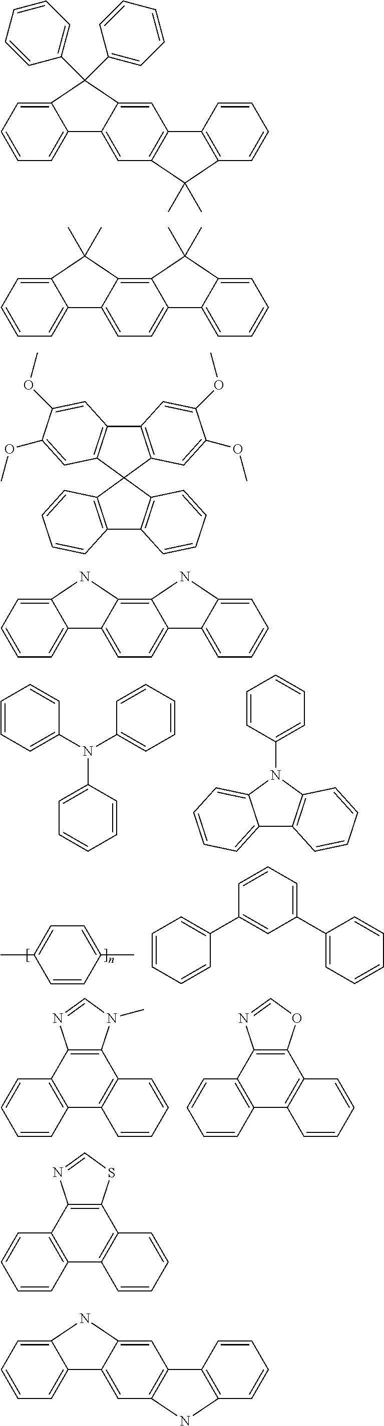

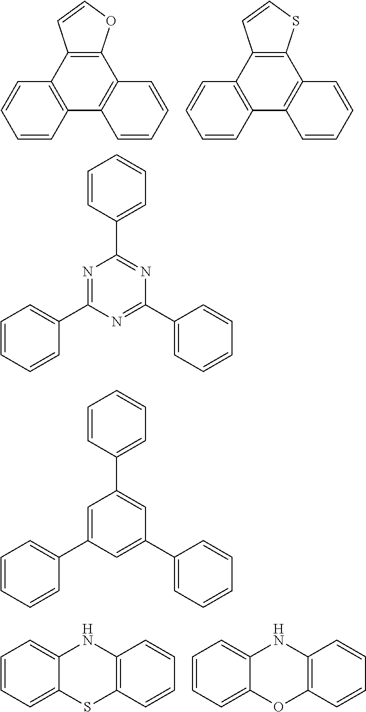

wherein n is 1, 2, 3, or 4.

In one embodiment, in the compound represented by the general formula (1) or the general formula (2), Y can be selected from: (1) C1˜C10 alkyl, C2˜C10 alkenyl, C2˜C10 alkynyl, and deuteride or fluoride thereof, such as from —CH₃, —CD₃, —CF₃, ethyl, n-propyl, isopropyl, cyclopropyl, n-butyl, isobutyl, sec-butyl, tert-butyl, cyclobutyl, 2-methylbutyl n-pentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl and octynyl; (2) C1˜C10 alkoxy and deuteride or fluoride thereof, such as from methoxy, ethoxy, n-propoxy, isopropoxy, n-butoxy, isobutoxy, sec-butoxy, tert-butoxy or 2-methylbutoxy; (3) C3˜C10 aryl, C2˜C10 heteroaryl, and deuteride or fluoride thereof, which may be univalent or bivalent according to use, in each case, may be substituted with the R¹ group as mentioned above, and may be linked to other aromatic rings or heteroaromatic rings on any desirable position, such as from: benzene, naphthalene, pyrene, dihydropyrene, chrysene, perylene, naphthacene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthaimidazole, phenanthroimidazole, imidazopyridine, imidazopyrazine, imidazoquinoxaline, oxazole, benzoxazole, naphthoxazole, anthraxoxazole, phenanthroxazole, isoxazole, 1,2-thiazole, 1,3-thiazole, benzothiazole, pyridazine, benzopyridazine, pyrimidine, benzopyrimidine, quinoxaline, pyrazine, 1,5-naphthyridine, nitrocarbazole, benzoporphyrin, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole. 1,2,4,5-tetrazine, 1,2,3,4-tetrazine, 1,2,3,5-tetrazine, purine, pteridine, indolizine, and benzothiadiazole. For the purpose of the present disclosure, except the aryl and heteroaryl as mentioned above, the aromatic ring and the heteroaromatic ring also refers to biphenylene, triphenylene, fluorene, spirobifluorene, dihydrophenanthrene, tetrahydropyrene, and cis-indenofluorene or trans-indenofluorene.

The thermally activated delayed fluorescence material claimed by the present disclosure has the thermally activated delayed fluorescence (TADF) property. In accordance with the principle of the thermally activated delayed fluorescence (TADF) material (see Adachi et al., Nature Vol 492, 234, (2012)), a triplet exciton of the thermally activated delayed fluorescence material can be reversely internally converted into a singlet exciton when the ΔE(S₁−T₁) of the organic compound is small enough, thereby obtaining the high efficient luminescence. In general, the TADF material is obtained by linking the acceptor group to the donor group, and thus has a remarkable D-A structure.

The thermally activated delayed fluorescence material claimed by the present disclosure has a small energy difference ΔE(S₁−T₁) between the singlet state and the triplet state which is generally smaller than or equal to 0.30 eV. In one embodiment, ΔE(S₁−T₁) of the thermally activated delayed fluorescence material is smaller than or equal to 0.25 eV. In another embodiment, ΔE(S₁−T₁) of the thermally activated delayed fluorescence material is smaller than or equal to 0.20 eV. In another embodiment, ΔE(S₁−T₁) of the thermally activated delayed fluorescence material is smaller than or equal to 0.15 eV. In another embodiment, ΔE(S₁−T₁) of the thermally activated delayed fluorescence material is smaller than or equal to 0.10 eV. ΔE(S₁−T₁) of compounds with similar structure reported before (see H Huang, et al., Chem. Eur. J, Vol 19, 1828, (2013)) are all larger than 0.30 eV.

In one embodiment, in the compound represented by the general formula (1) or the general formula (2), at least one of Ar³ and Ar⁴ includes one donor group and/or one acceptor group.

In one embodiment, in the compound represented by the general formula (1), at least one of Ar³ and Ar⁴ includes one donor group when the sub-structure represented by the general formula (1-a) has the electron-withdrawing property. In one embodiment, Ar³ and Ar⁴ respectively include one donor group.

In one embodiment, the sub-structure represented by the general formula (1-a) with the electron-withdrawing property is selected from:

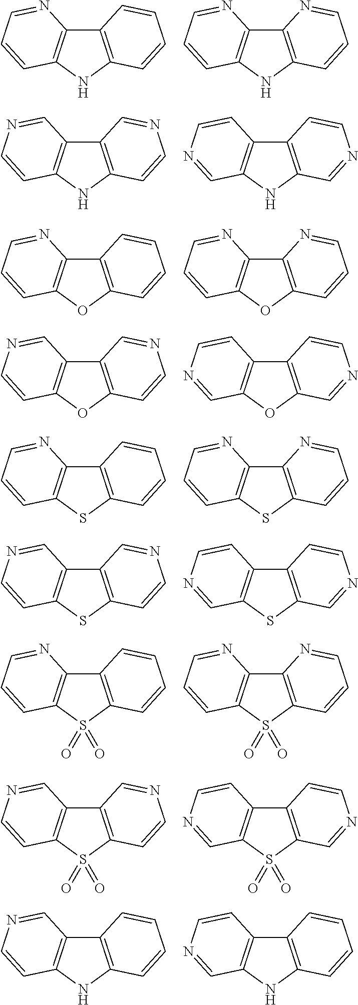

In one embodiment, in the compound represented by the general formula (1), at least one of Ar³ and Ar⁴ includes one acceptor group when the sub-structure represented by the general formula (1-a) has the electron-donating property. In one embodiment, Ar³ and Ar⁴ respectively includes one acceptor group.

In one embodiment, the sub-structure represented by the general formula (1-a) with the electron-donating property is selected from:

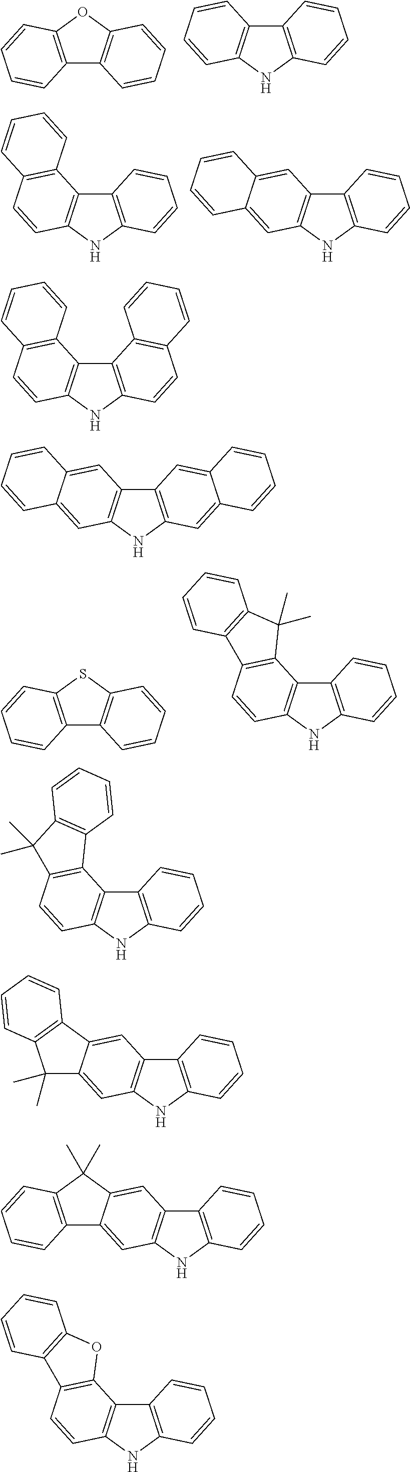

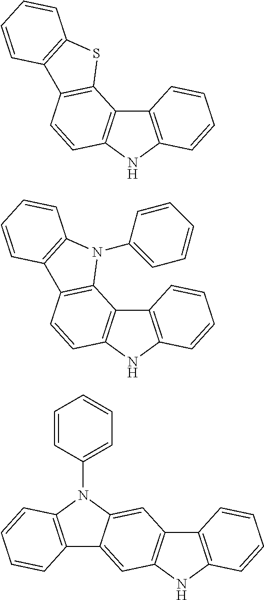

In one embodiment, in the compound represented by the general formula (1), one of Ar³ and Ar⁴ includes at least one donor group, and the other one of Ar³ and Ar⁴ includes at least one acceptor group.

The donor group as mentioned above can be selected from a structure including the following groups:

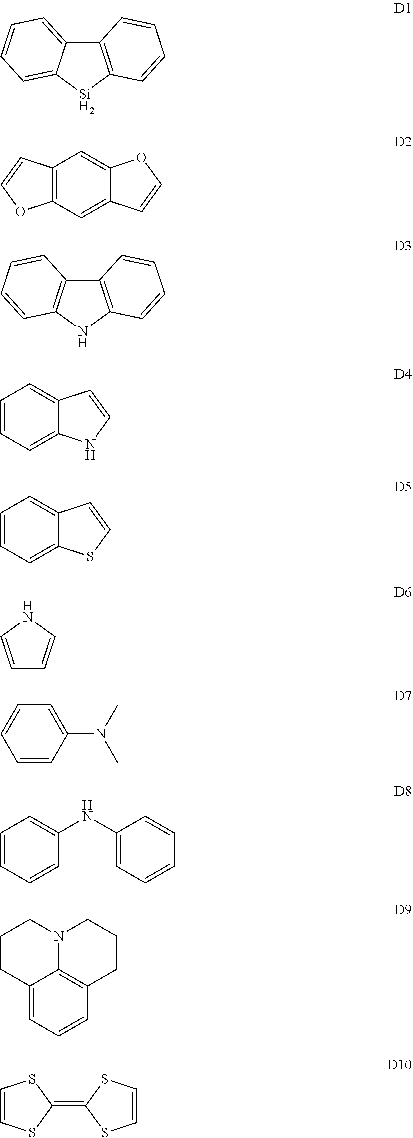

The acceptor group as mentioned above can be selected from F, cyano group, or a structure including the following groups:

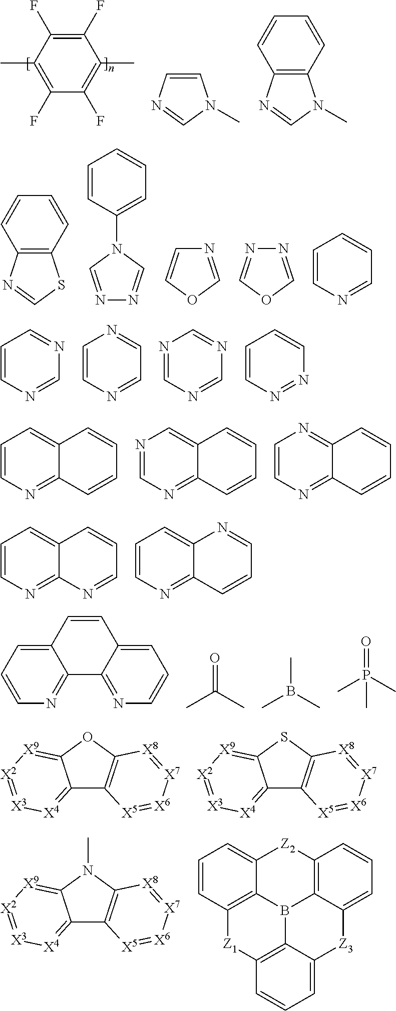

wherein n is 1, 2, or 3;

X²-X⁹ is selected from CR or N, at least one of which is N;

Z₁, Z₂, and Z₃ each independently represents N(R), C(R)₂, Si(R)₂, O, C═N(R), C═C(R)₂, P(R), P(═O)R, S, S═O, SO₂, or nothing, at least one of which is not nothing;

R is selected from hydrogen, alkyl, alkoxy, amino, alkenyl, alkynyl, aralkyl, heteroaralkyl, aryl, and heteroaryl.

In one embodiment, the thermally activated delayed fluorescence material of the present disclosure is a small molecule material.

As used herein, the term “small molecule” refers to a molecule that is not a polymer, oligomer, dendrimer, or polymer blend. In particular, there is no repetitive structure in the small molecule. The molecular weight of the small molecule is smaller than or equal to 4000 g/mol. In one embodiment, the molecular weight of the small molecule is smaller than or equal to 3000 g/mol. In another embodiment, the molecular weight of the small molecule is smaller than or equal to 2000 g/mol.

Polymer includes homopolymer, copolymer, block copolymer, and dendrimer in the present disclosure. The synthesis and application of dendrimer are described in Dendrimers and Dendrons, Wiley-VCH Verlag GmbH & Co. KGaA, 2002, Ed. George R. Newkome, Charles N. Moorefield, Fritz Vogtle.

Conjugated polymer is a polymer whose backbone is primarily composed by the sp² hybrid orbital of carbon atoms, some known examples include polyacetylene and poly (phenylene vinylene). The carbon atoms in the backbone of the conjugated polymer may also be substituted with other non-carbon atoms, and it is still considered to be a conjugated polymer even when the sp² hybridization on the backbone is broken by some natural defects. In addition, the conjugated polymer in the present disclosure may also include the polymer whose backbone contains aryl amine, aryl phosphine, other heteroaromatics, organometallic complexes, and etc.

The solubility of the organic small molecule compound can be ensured by the substituents on the structure unit of the general formula (1), the general formula (2), or any one of the general formulas (3)-(26) or on any benzene ring unit. Other substituents, if any, may also help to improve the solubility.

Depending on types of the substituents, the structure unit of the general formula (1), general formula (2), or any one of general formulas (3)-(26) may be suitable for various functions of the organic small molecule compound, thus may be used as a main skeleton of the small molecule compound or as an emitter. The substituent Y determines the correspondence of the compounds to the functions. Ar₃ and Ar₄, and R₁₀ and R₁₁ have an impact on the electronic property of the structure unit of the general formula (1), general formula (2), or any one of general formulas (3)-(26).

In one embodiment, structure formulas of the compounds represented by the general formula (1) are as follows, which may be further optionally substituted on any possible position:

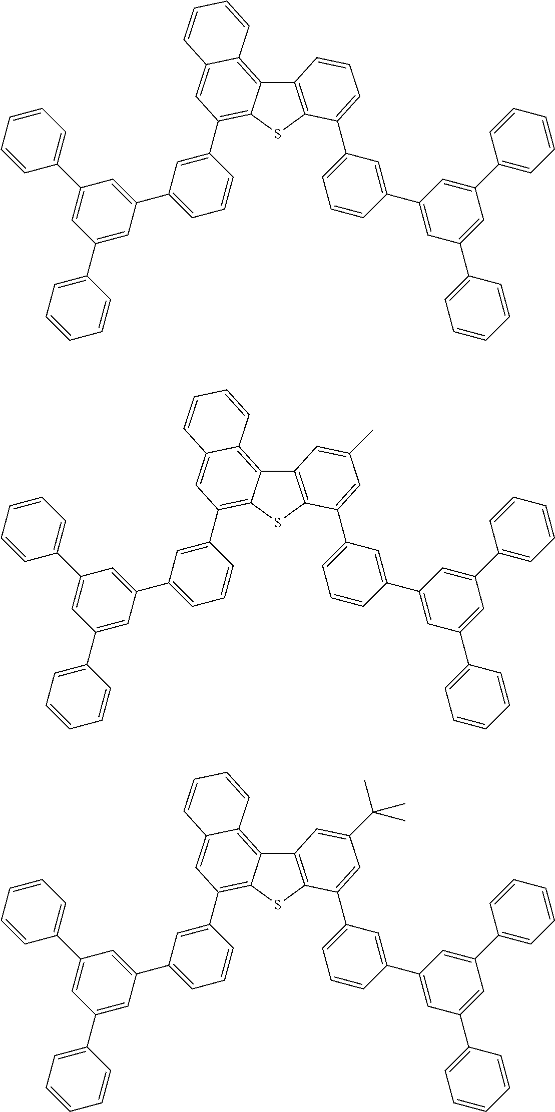

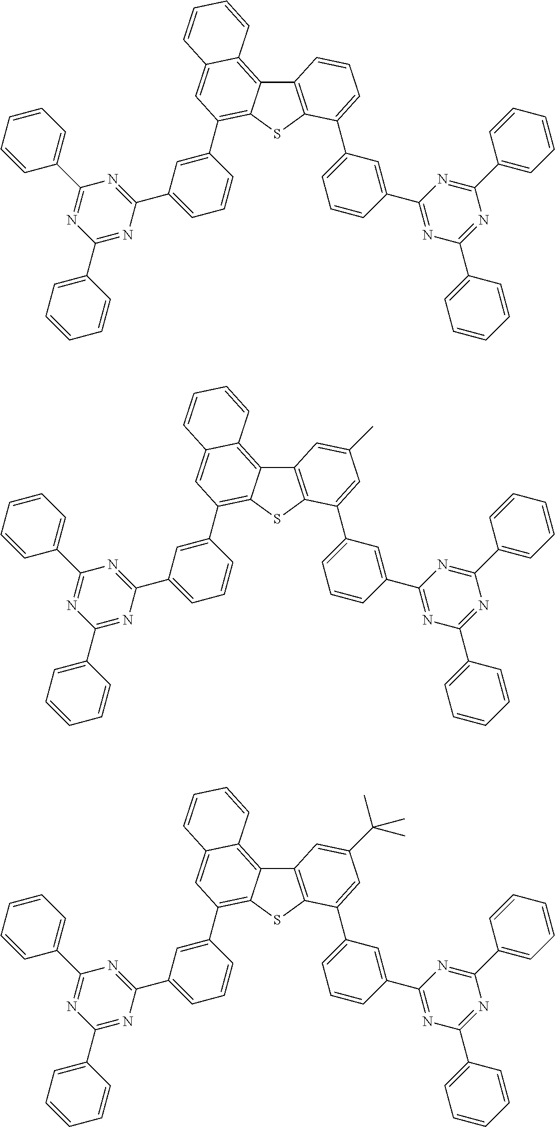

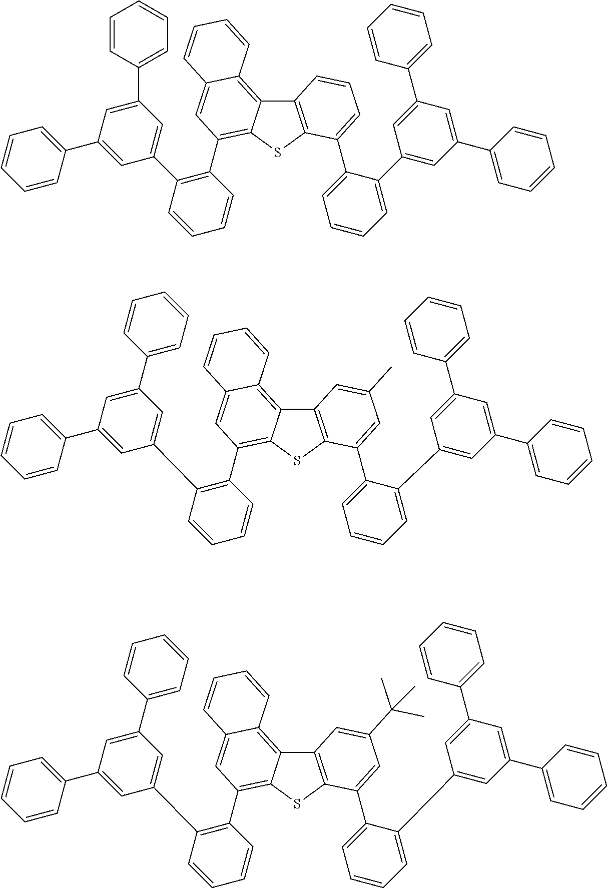

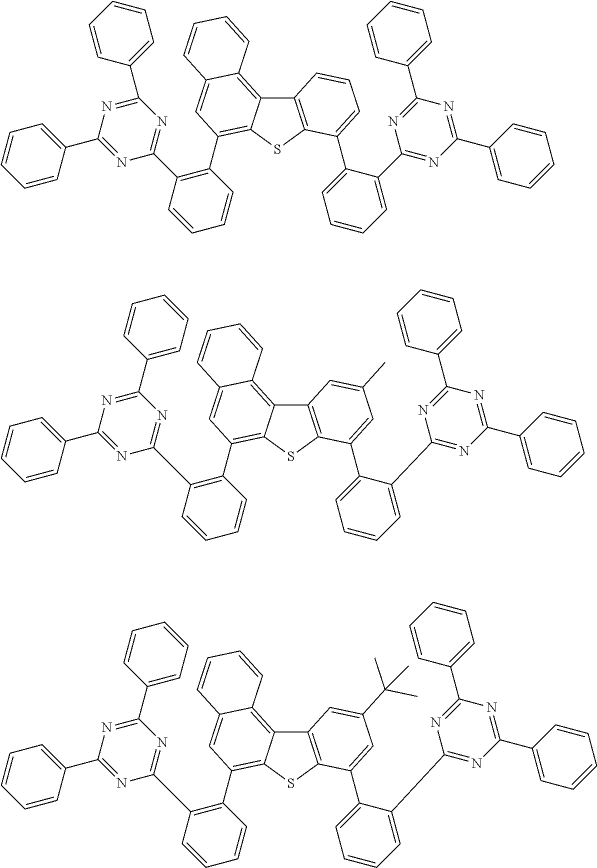

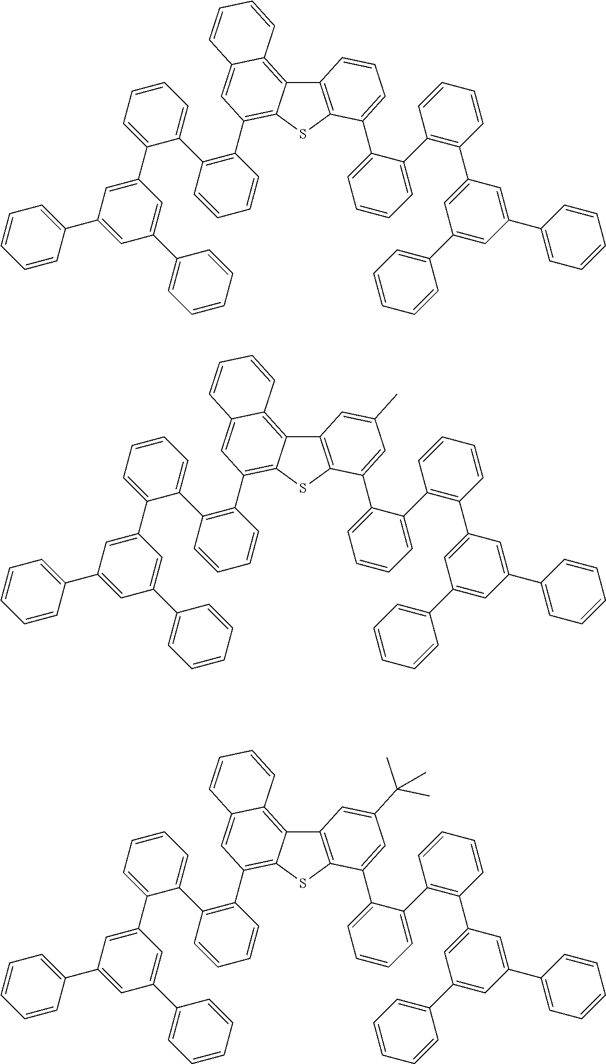

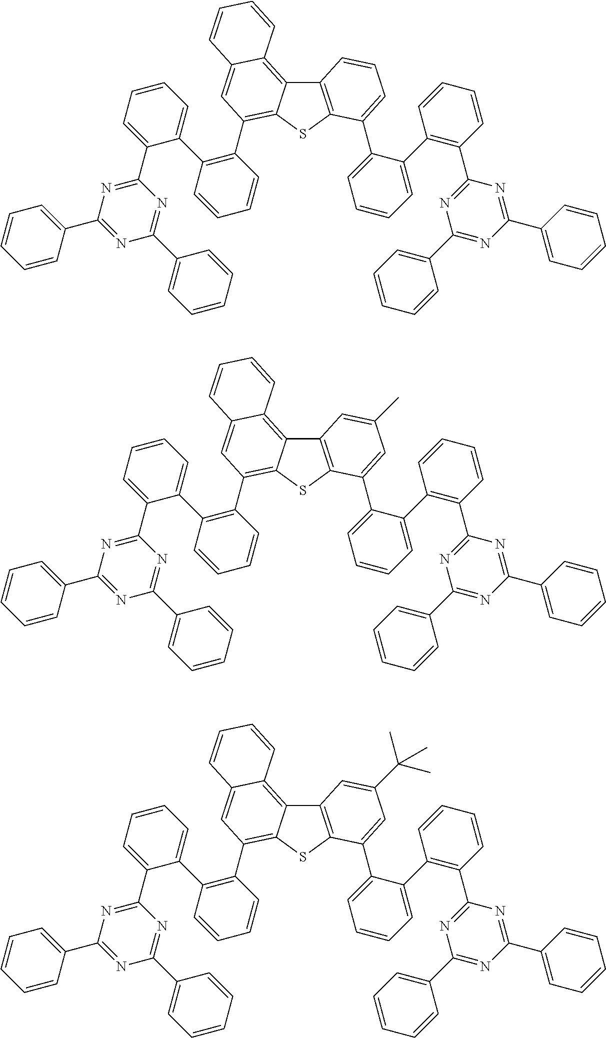

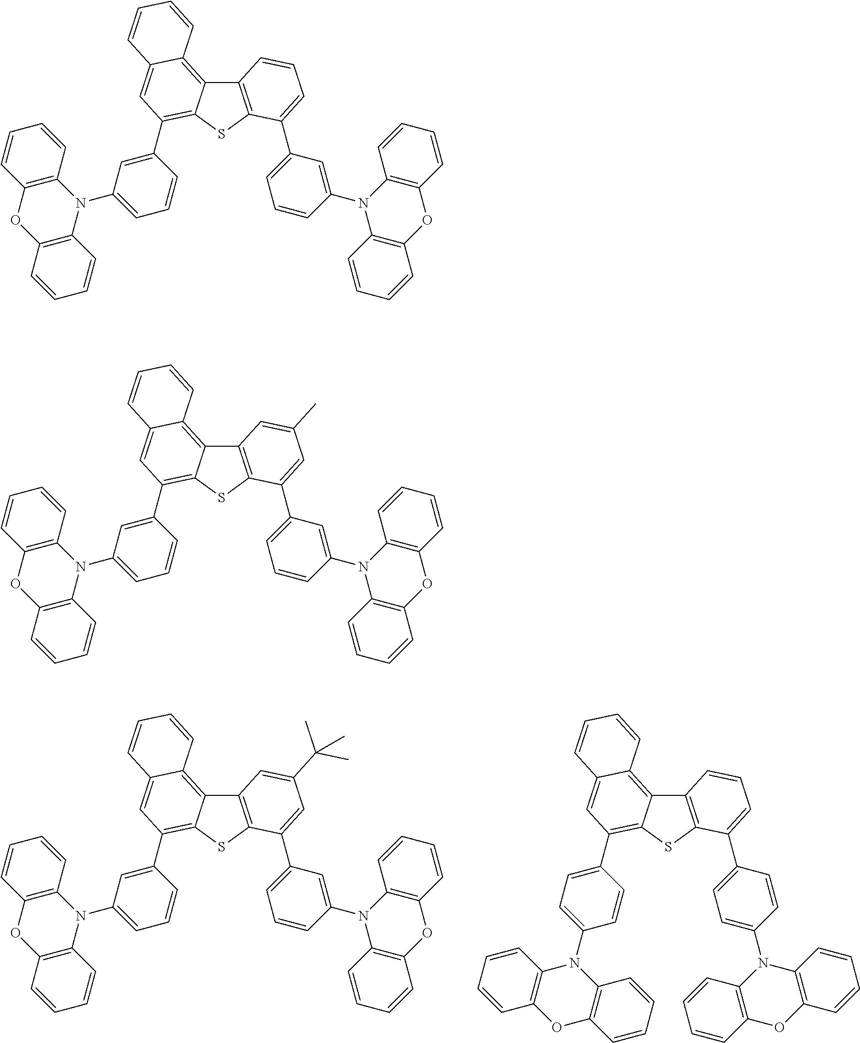

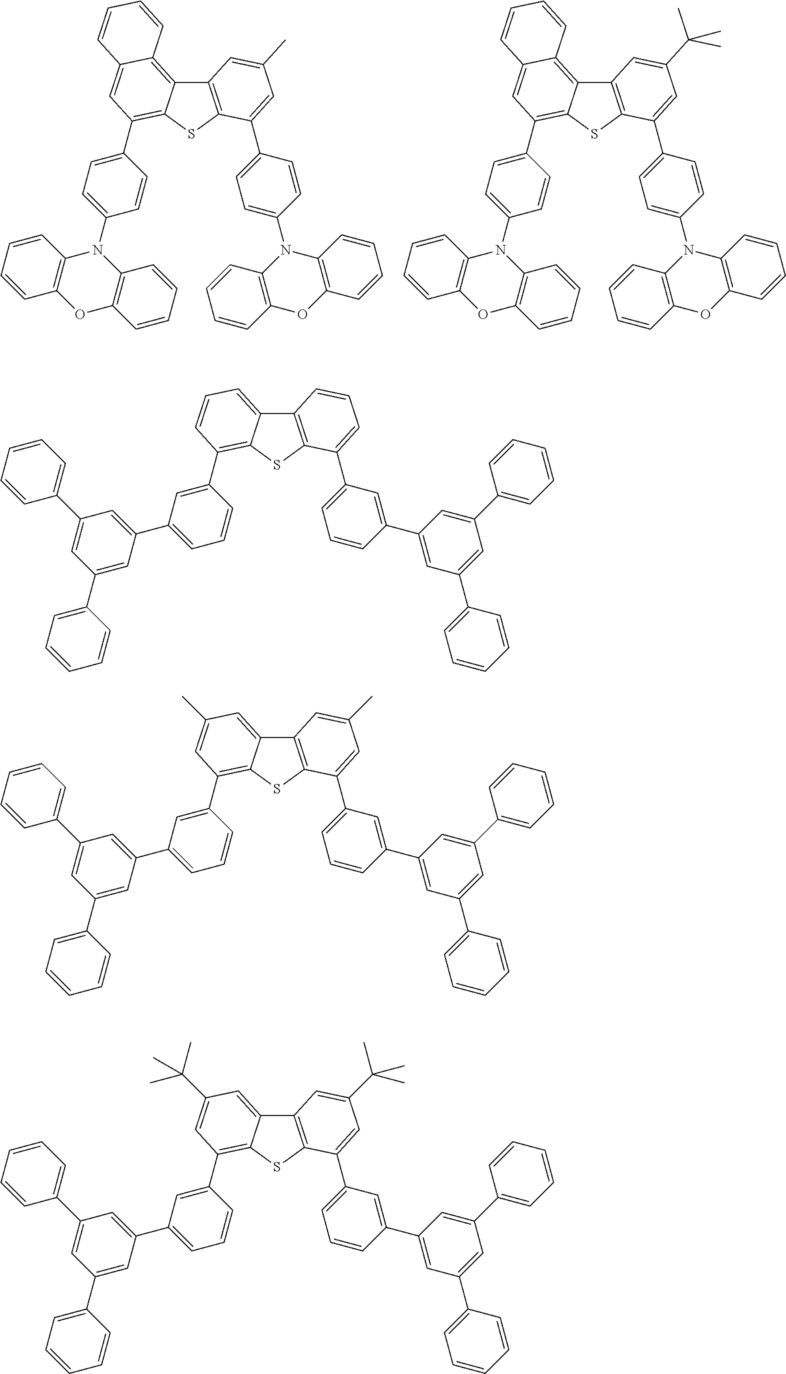

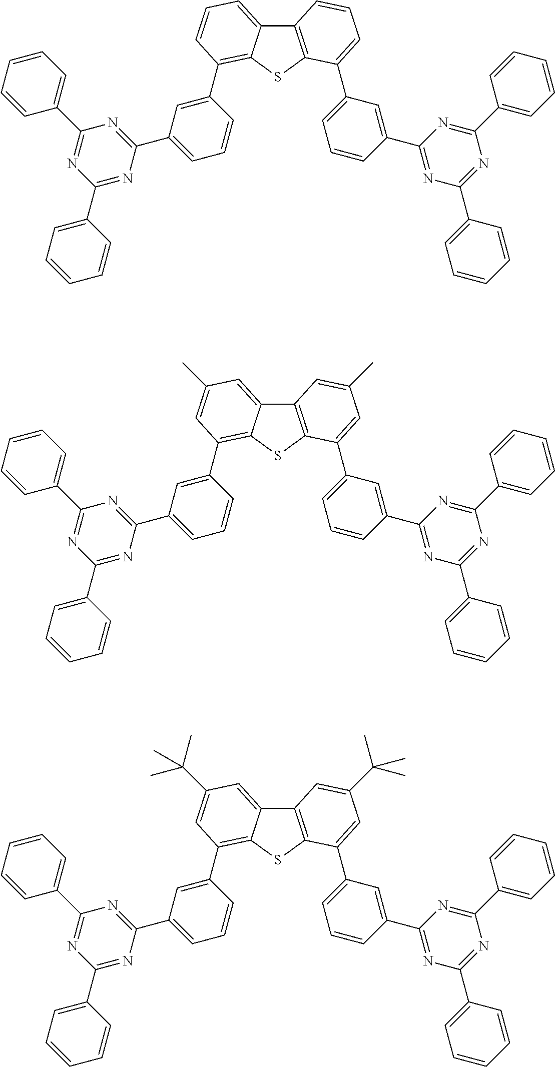

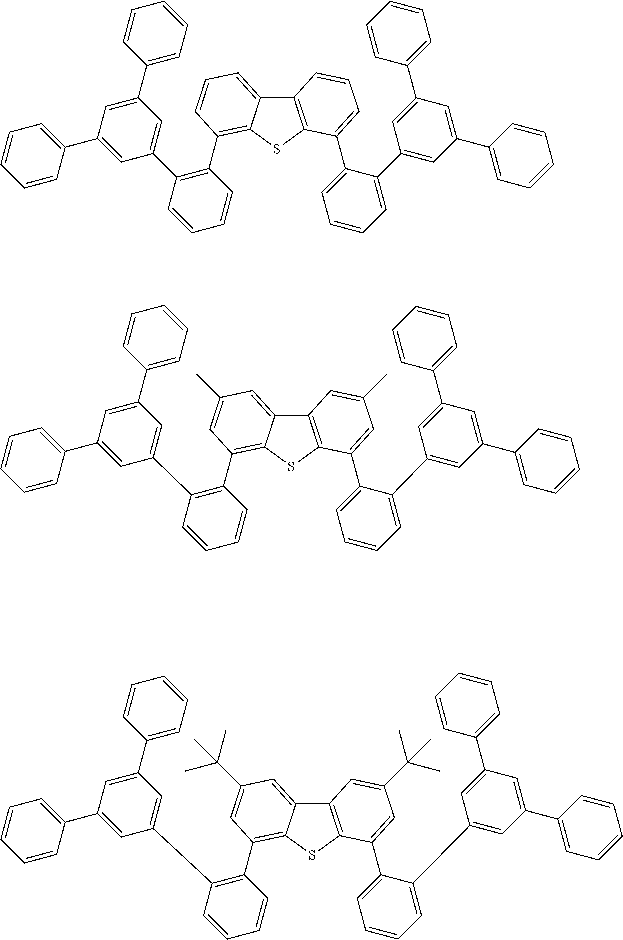

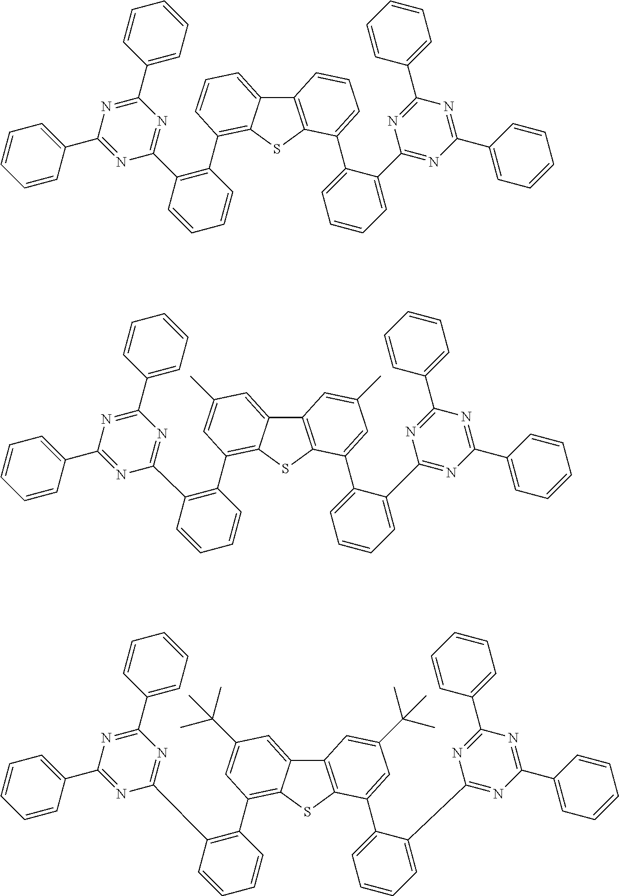

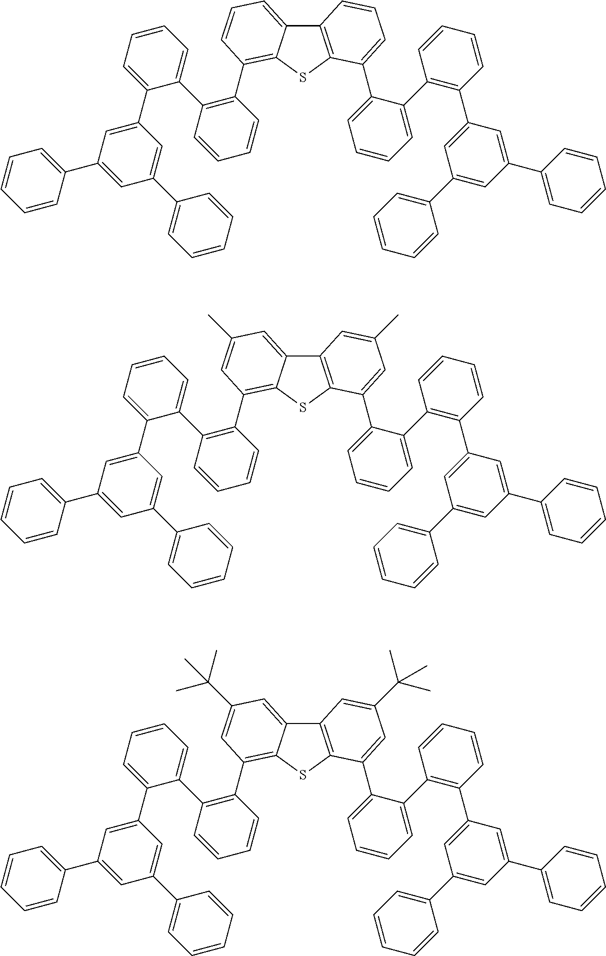

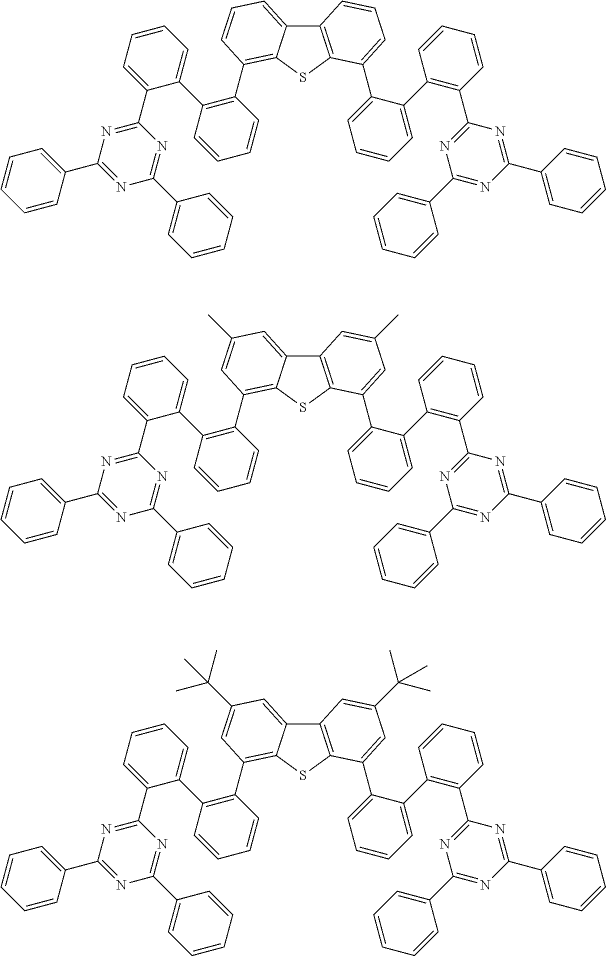

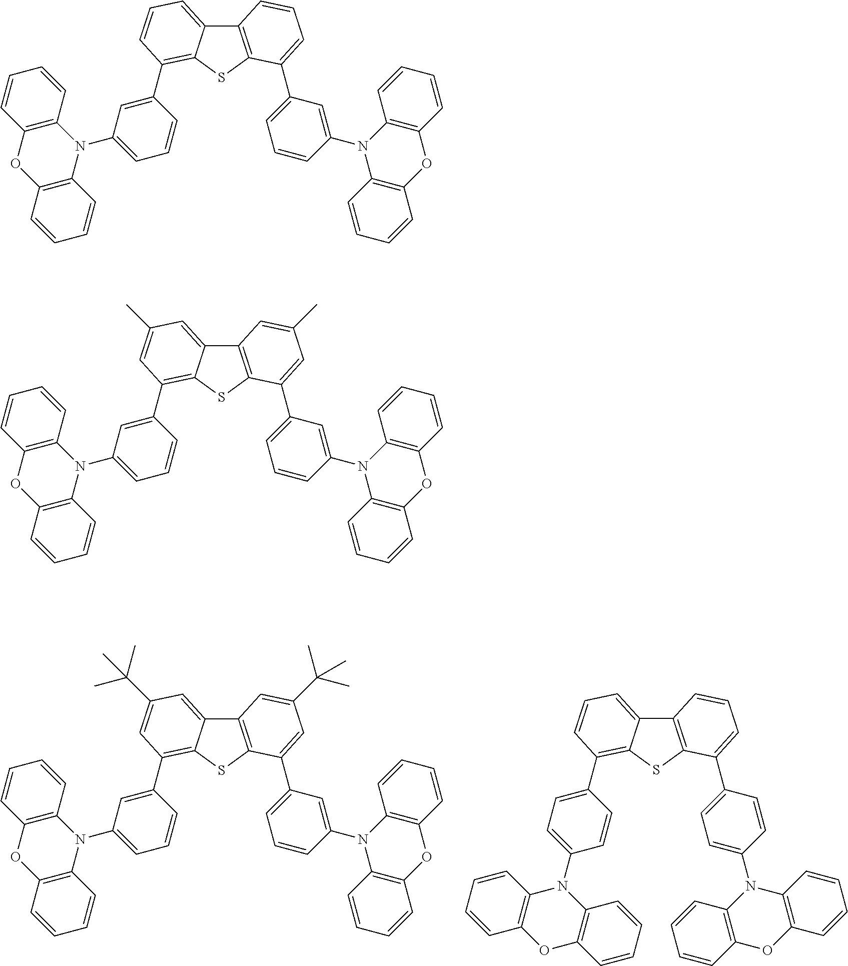

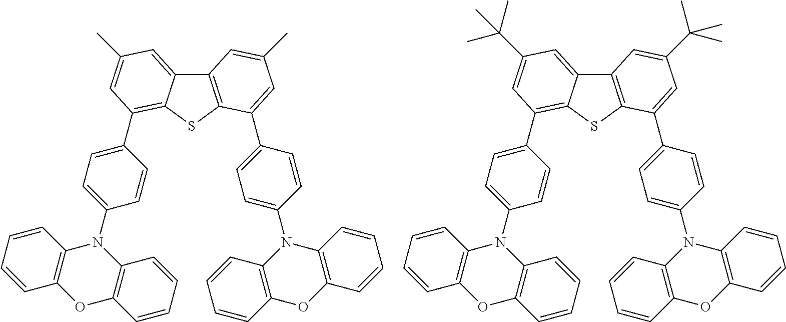

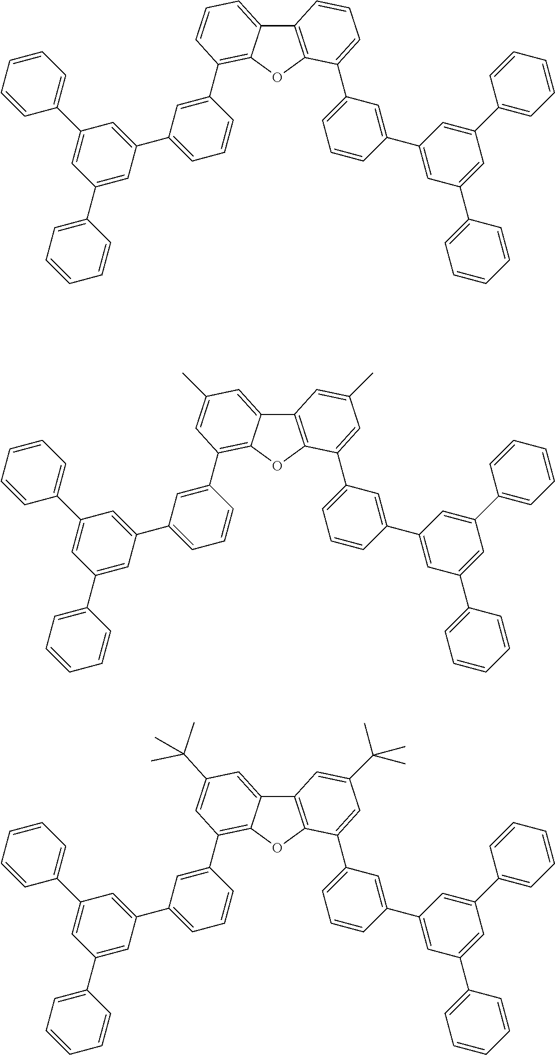

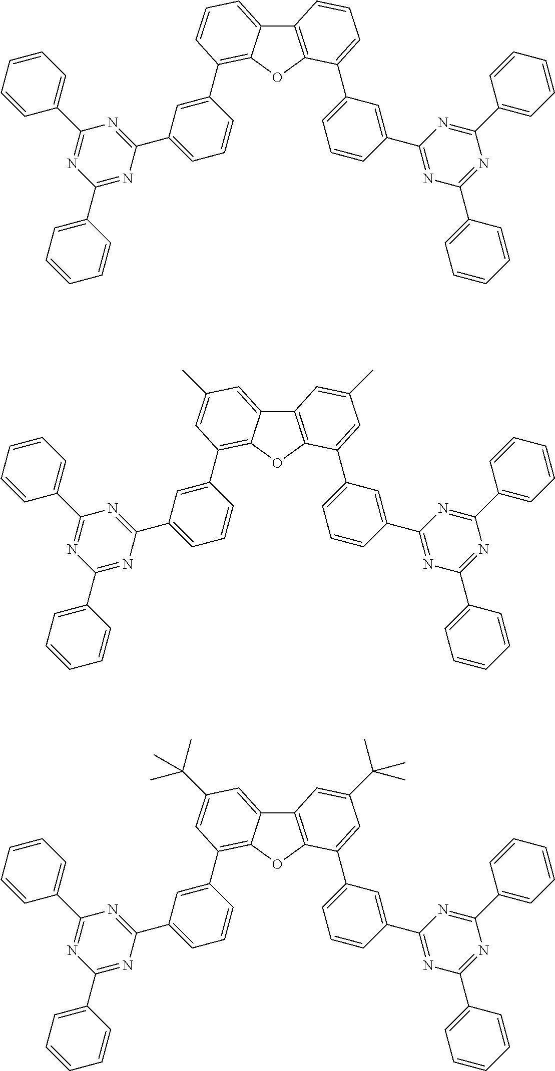

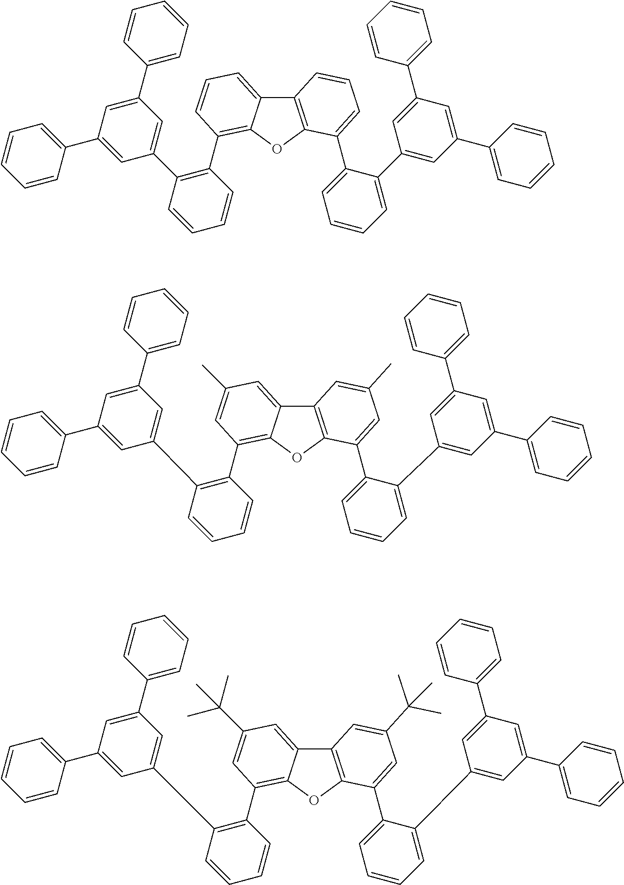

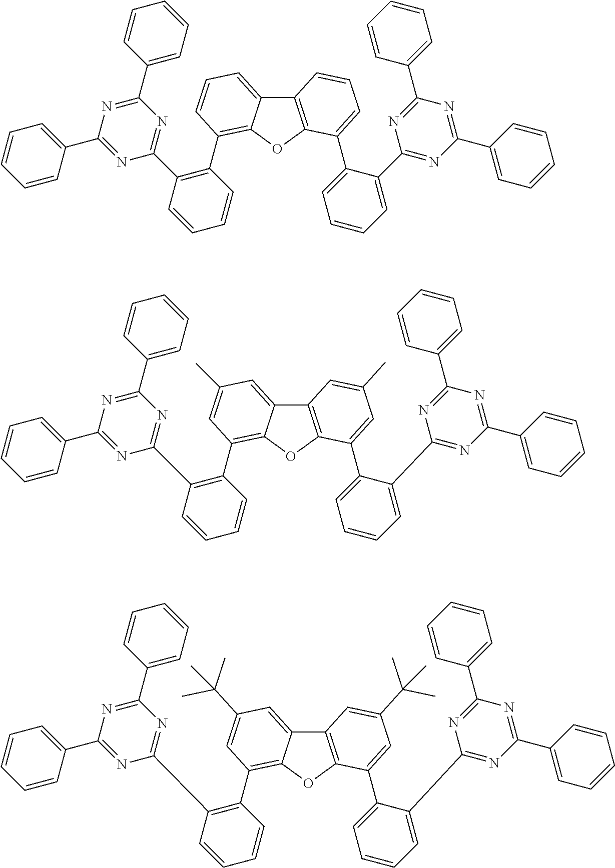

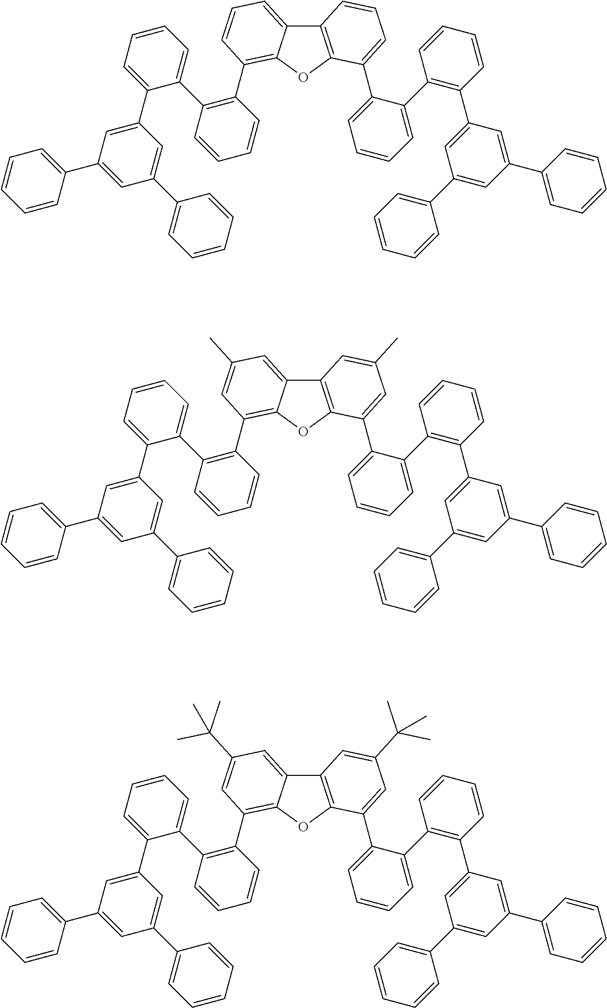

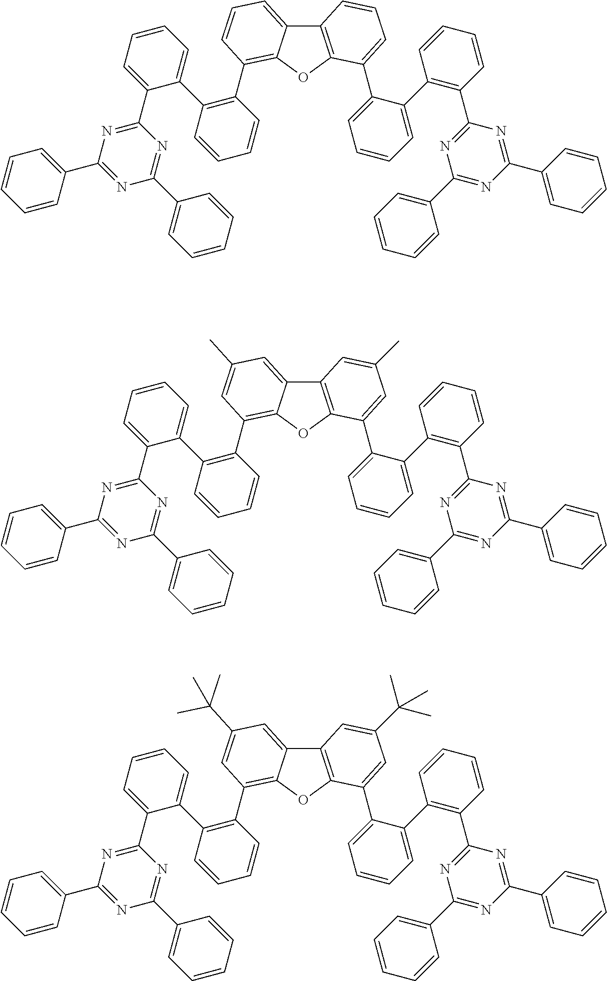

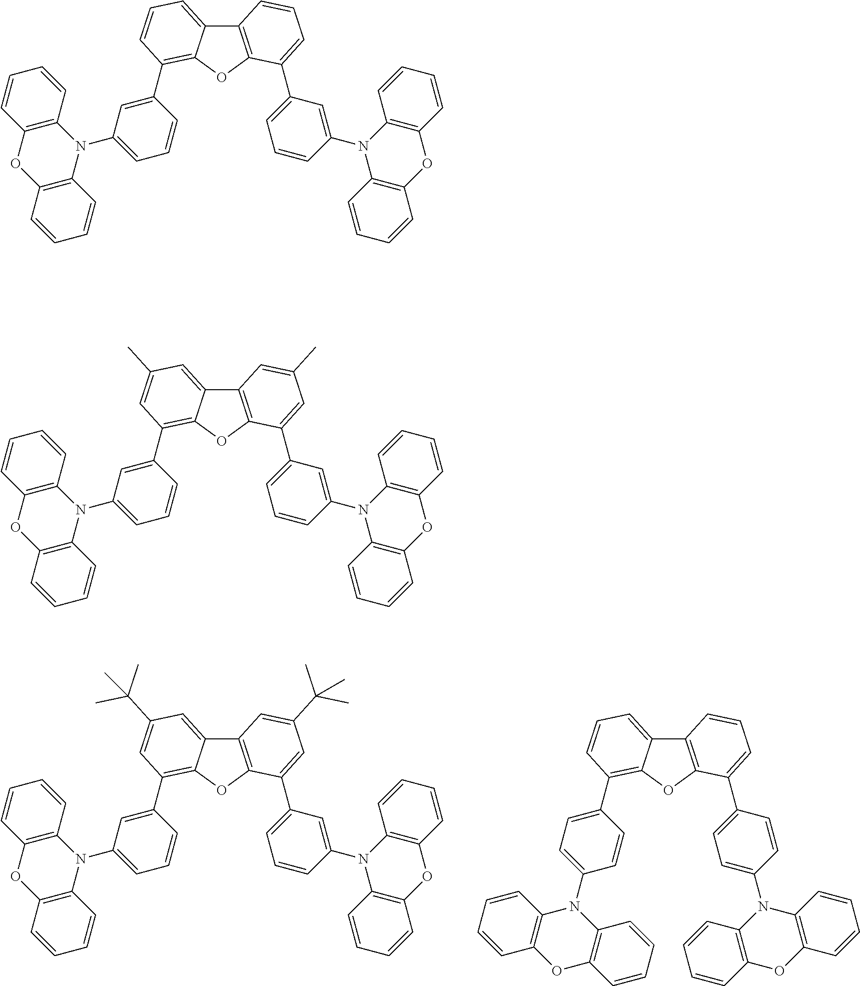

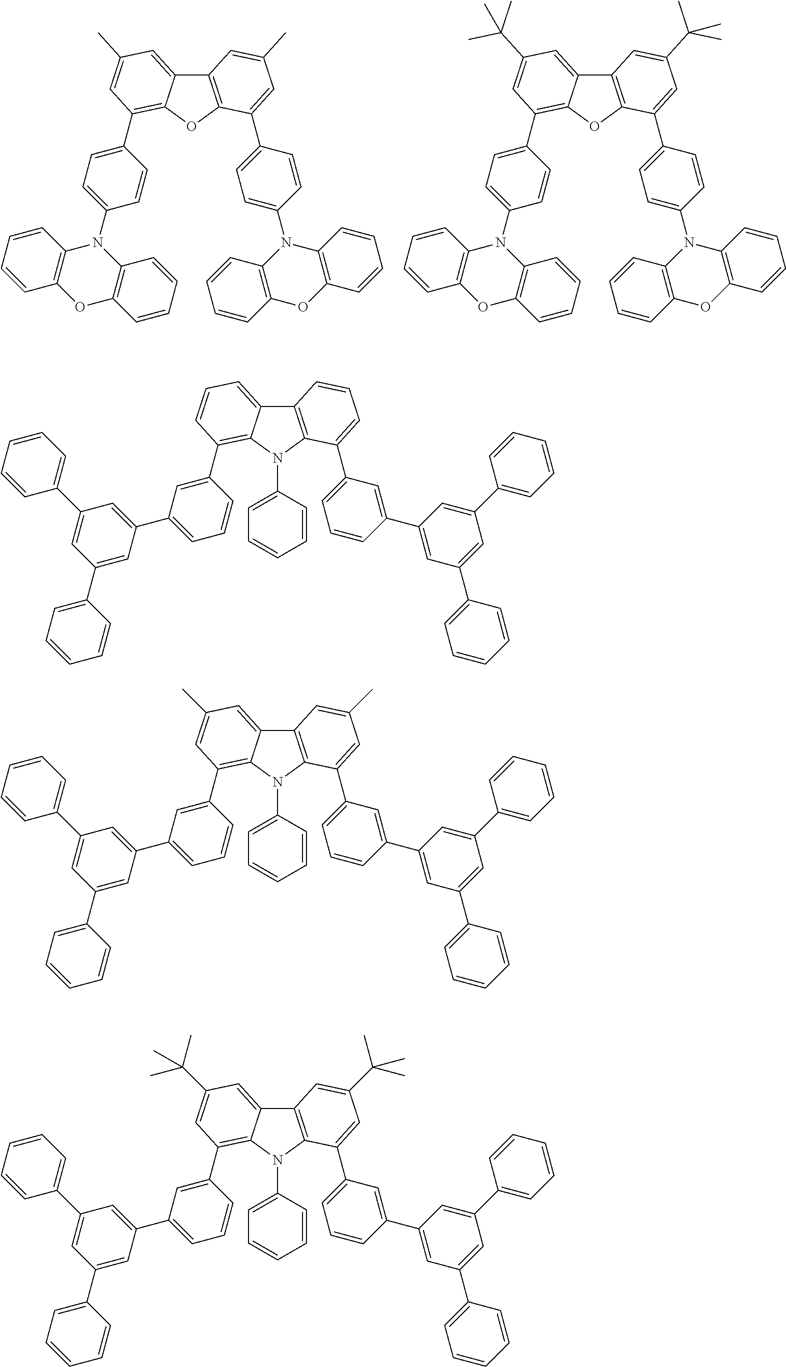

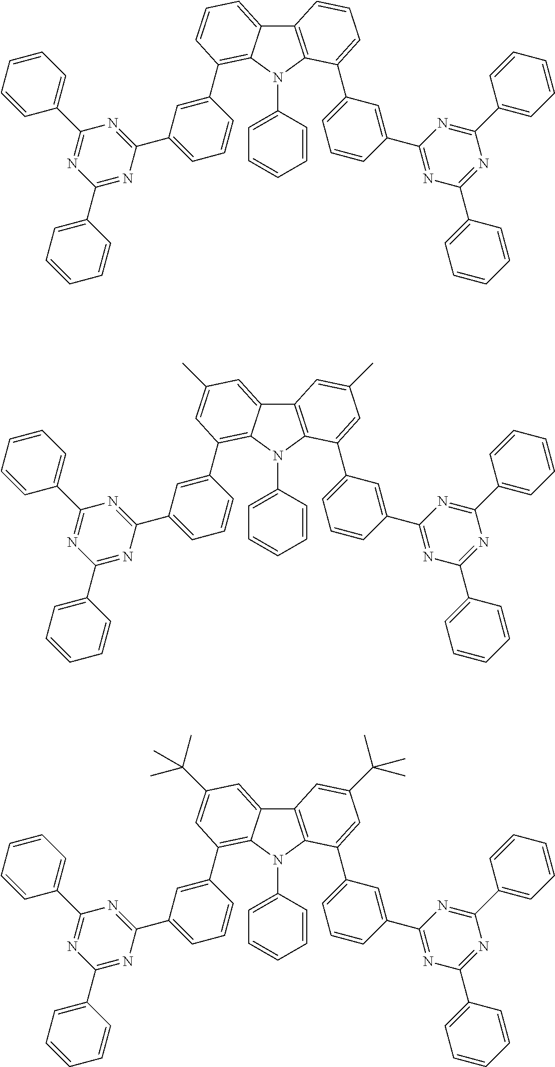

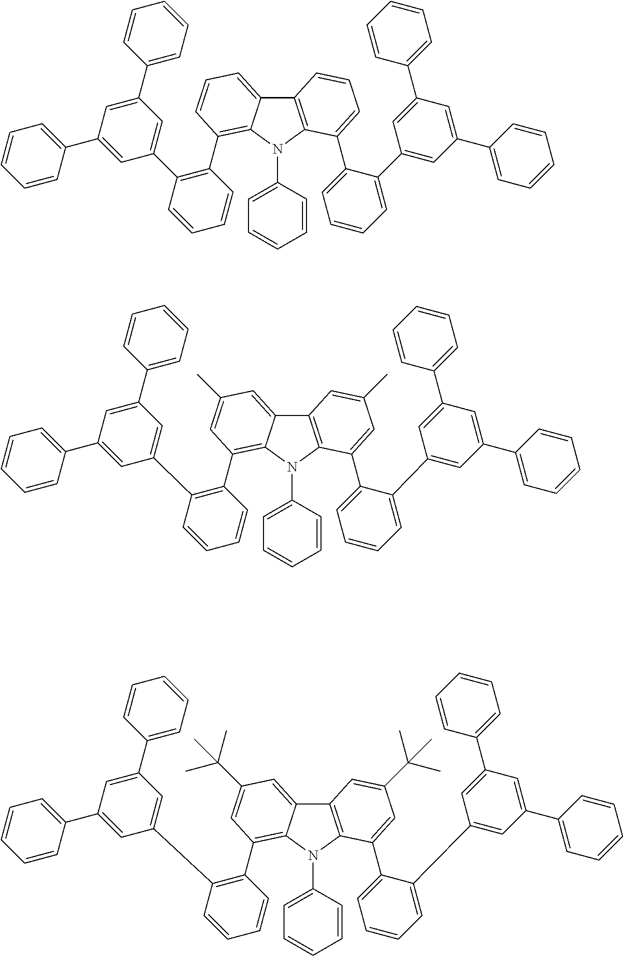

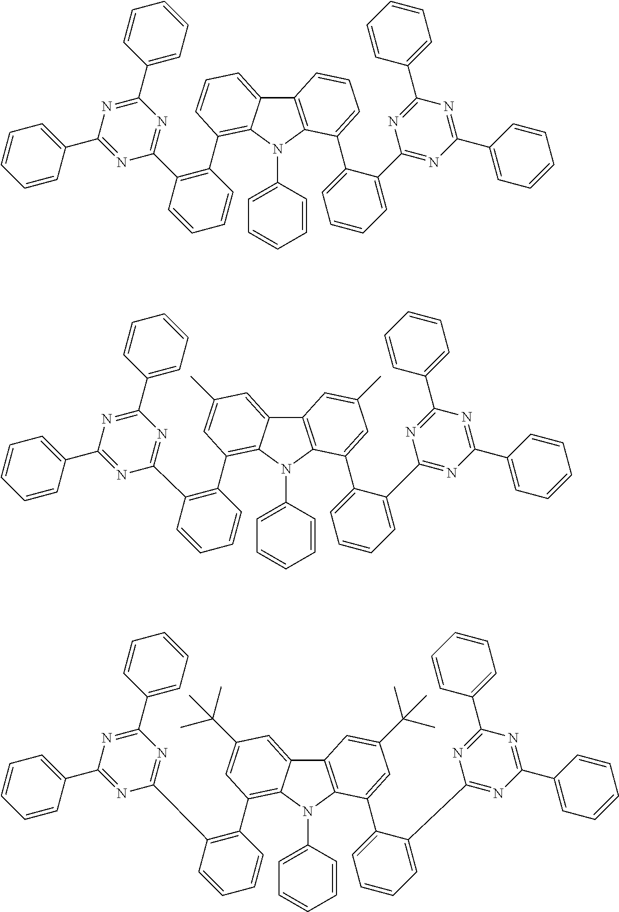

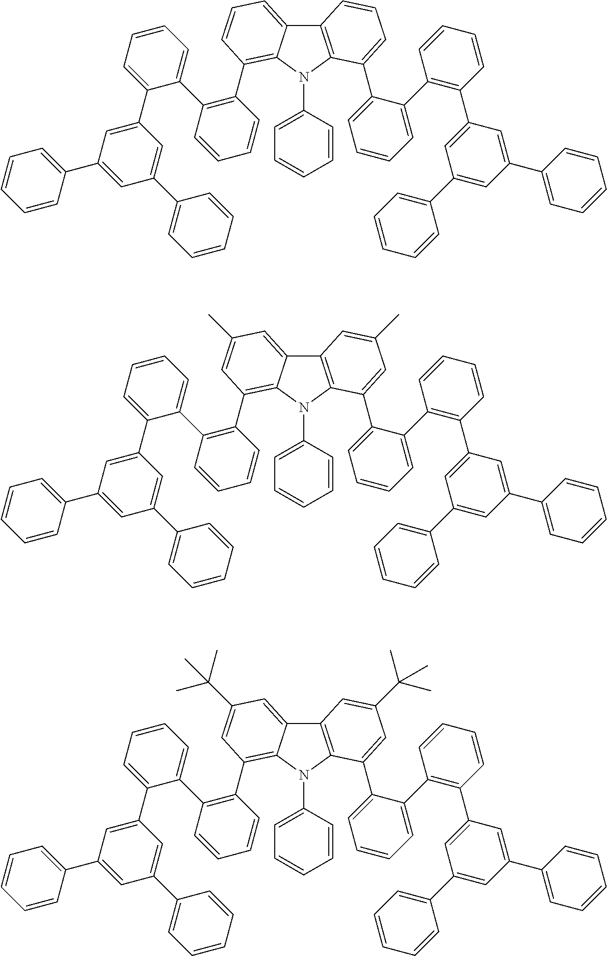

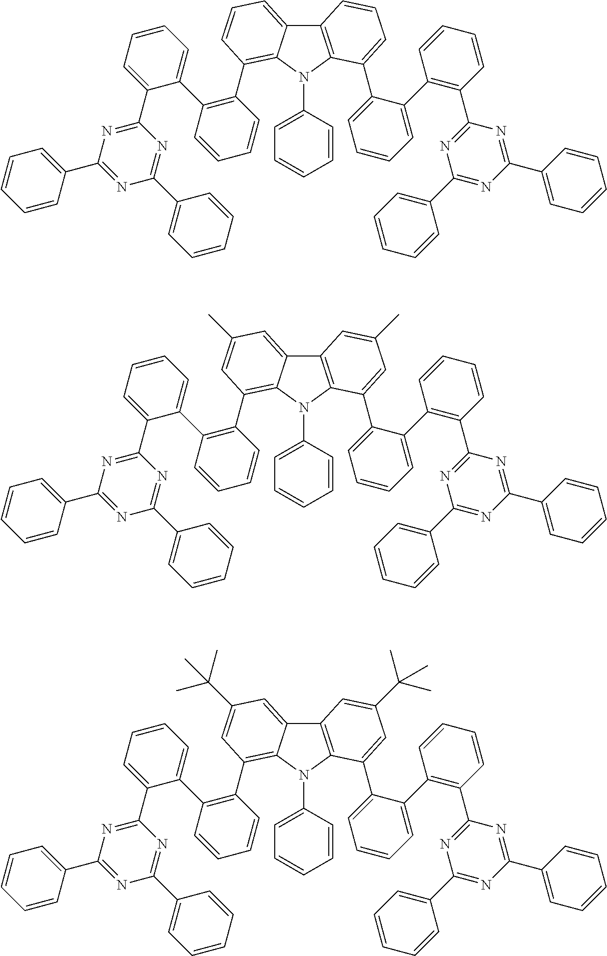

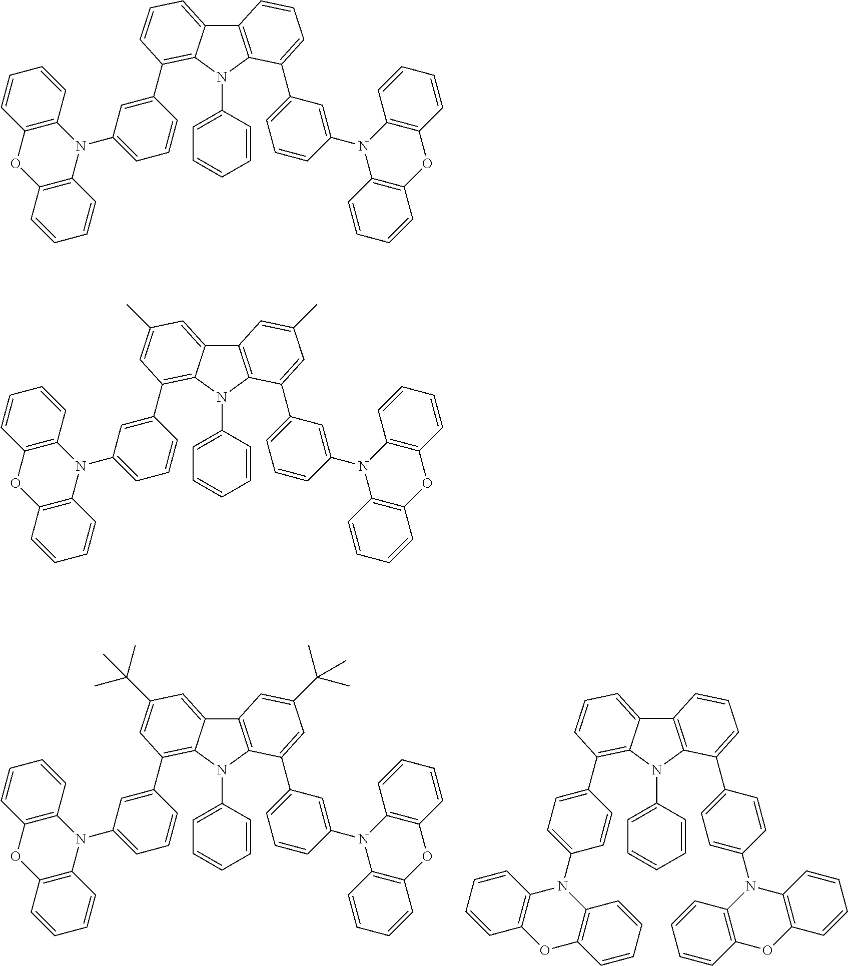

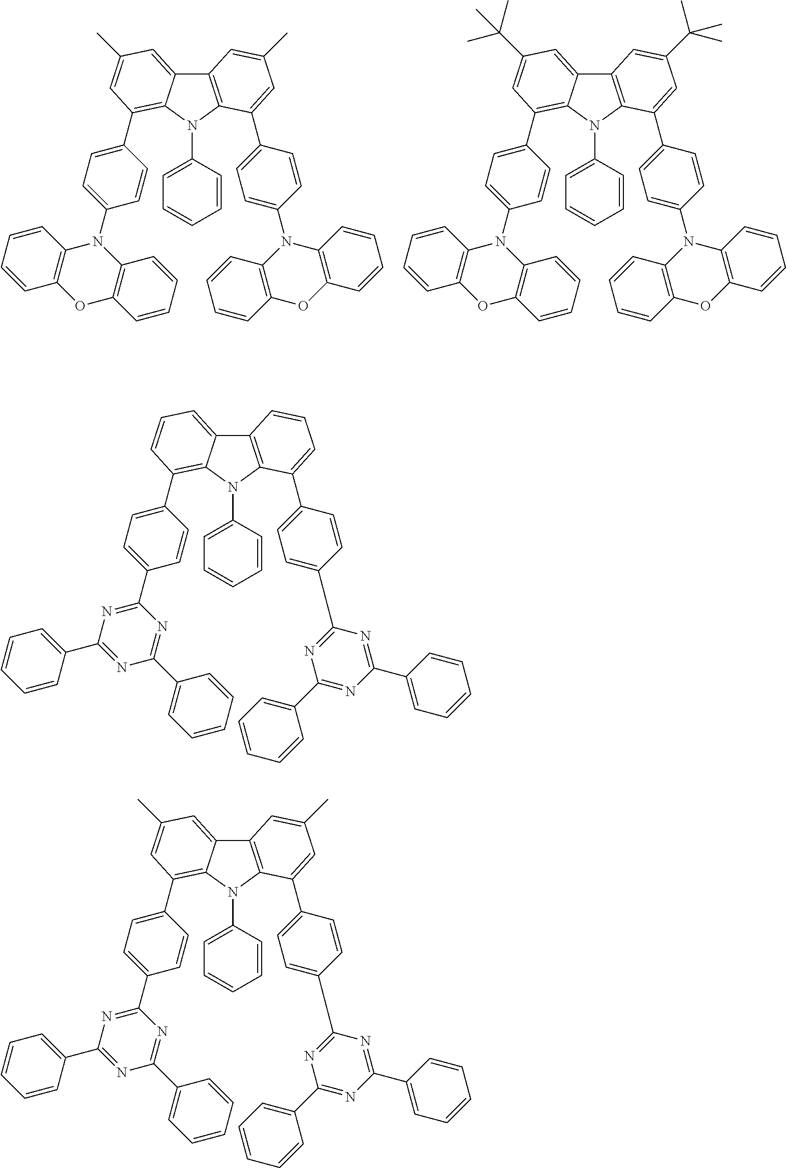

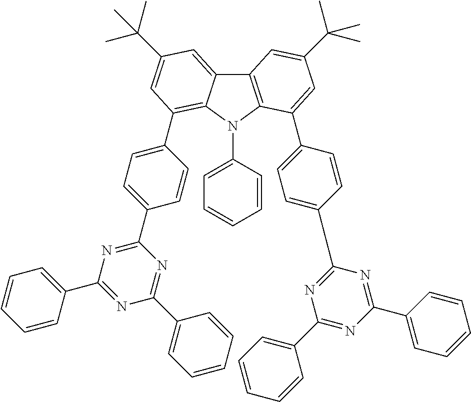

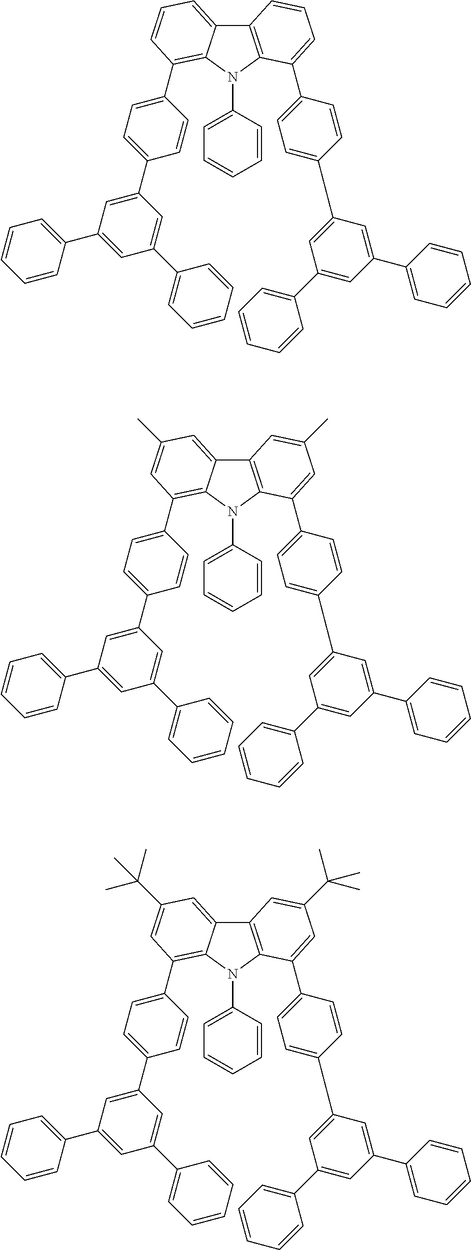

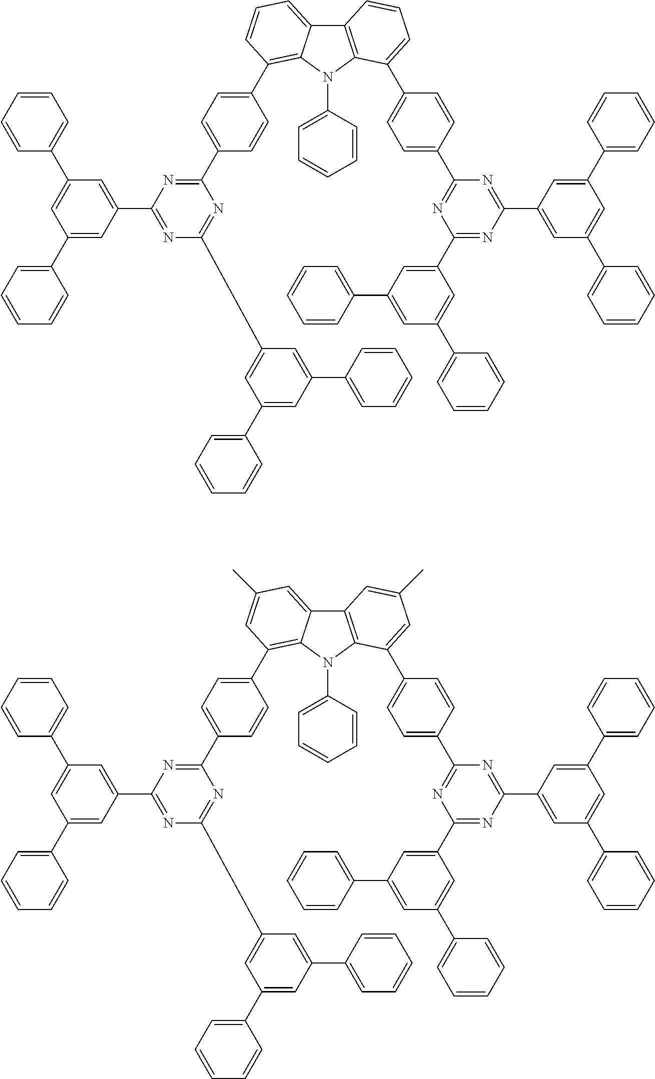

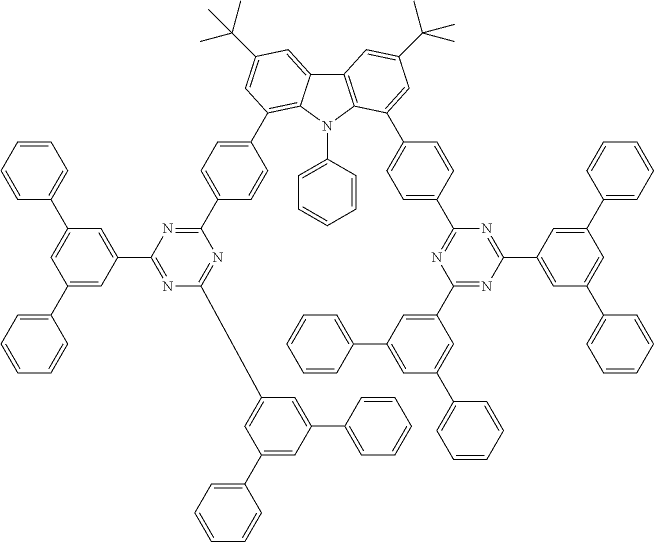

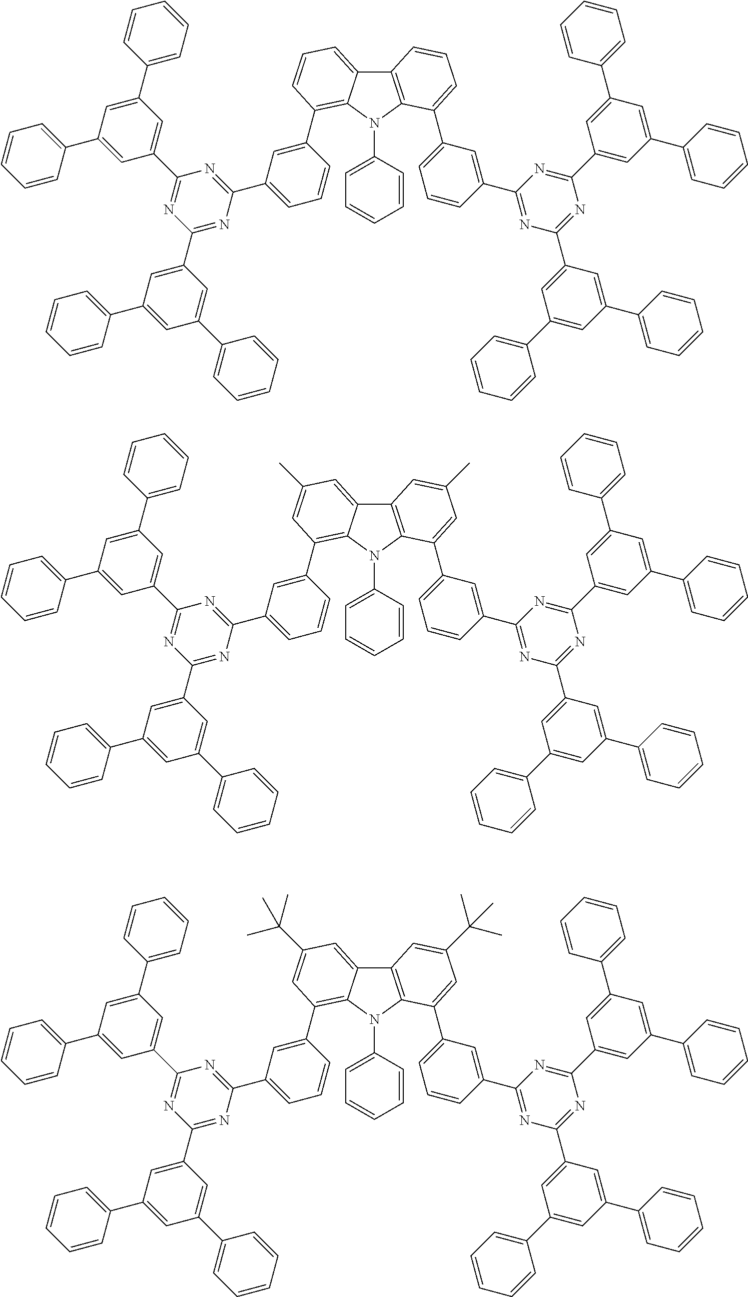

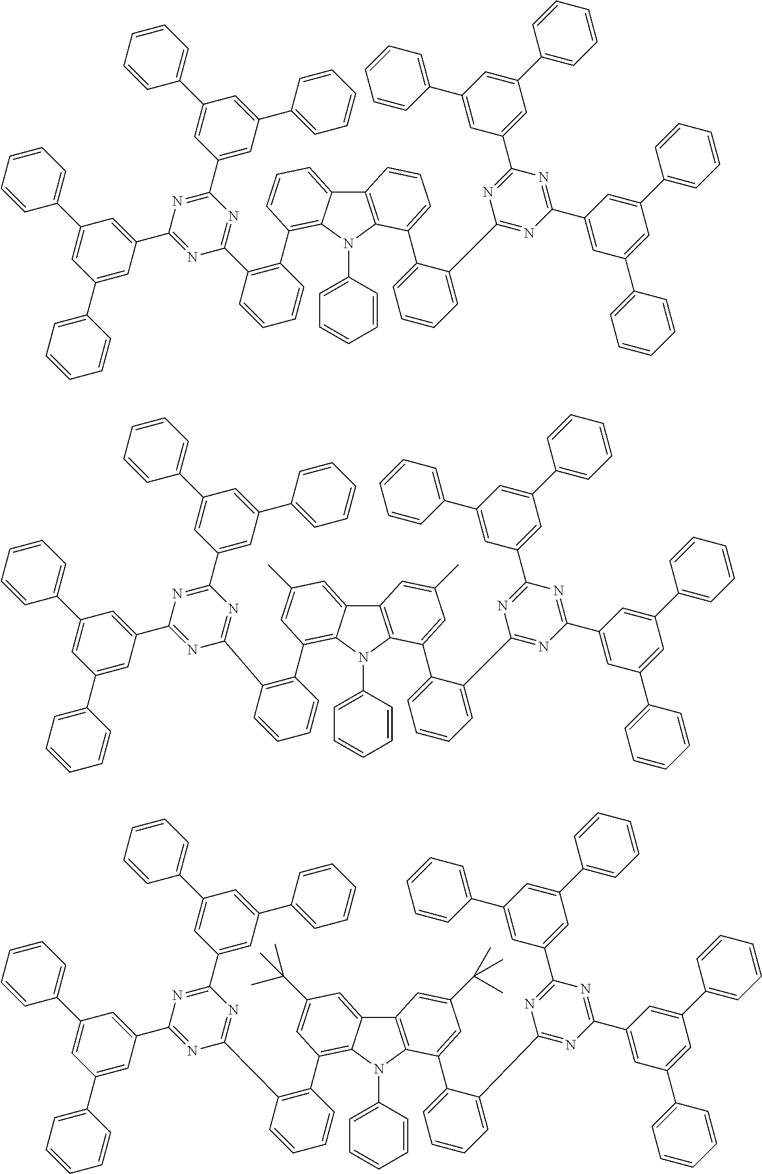

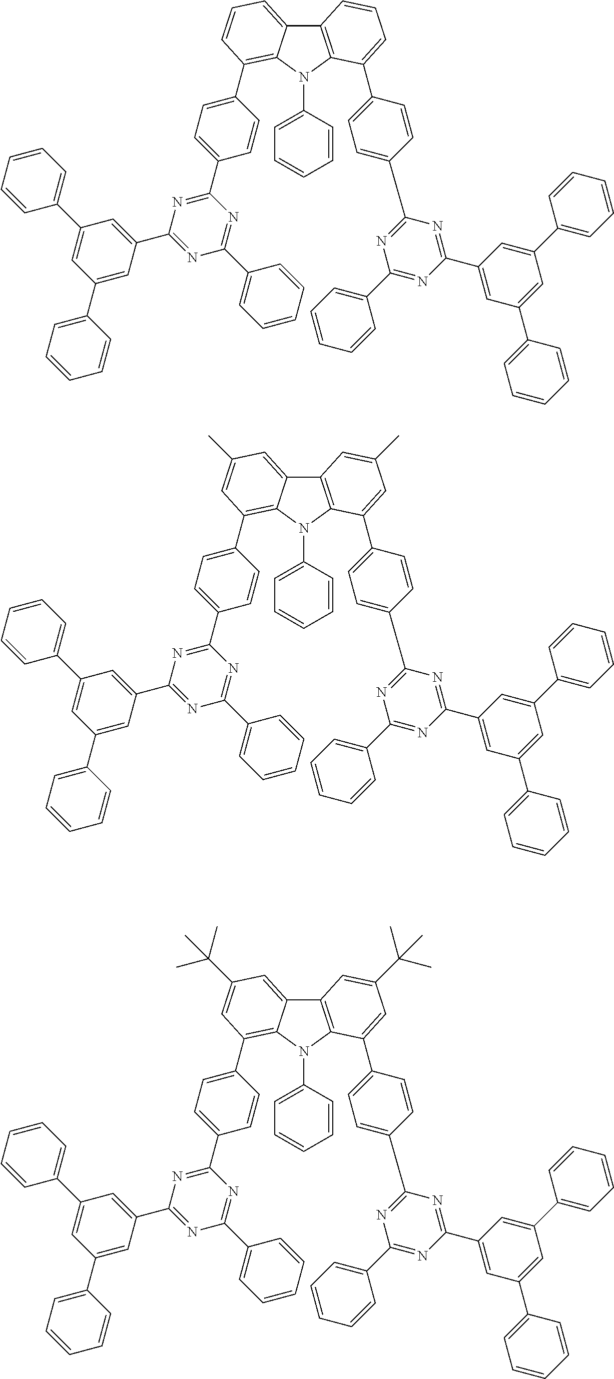

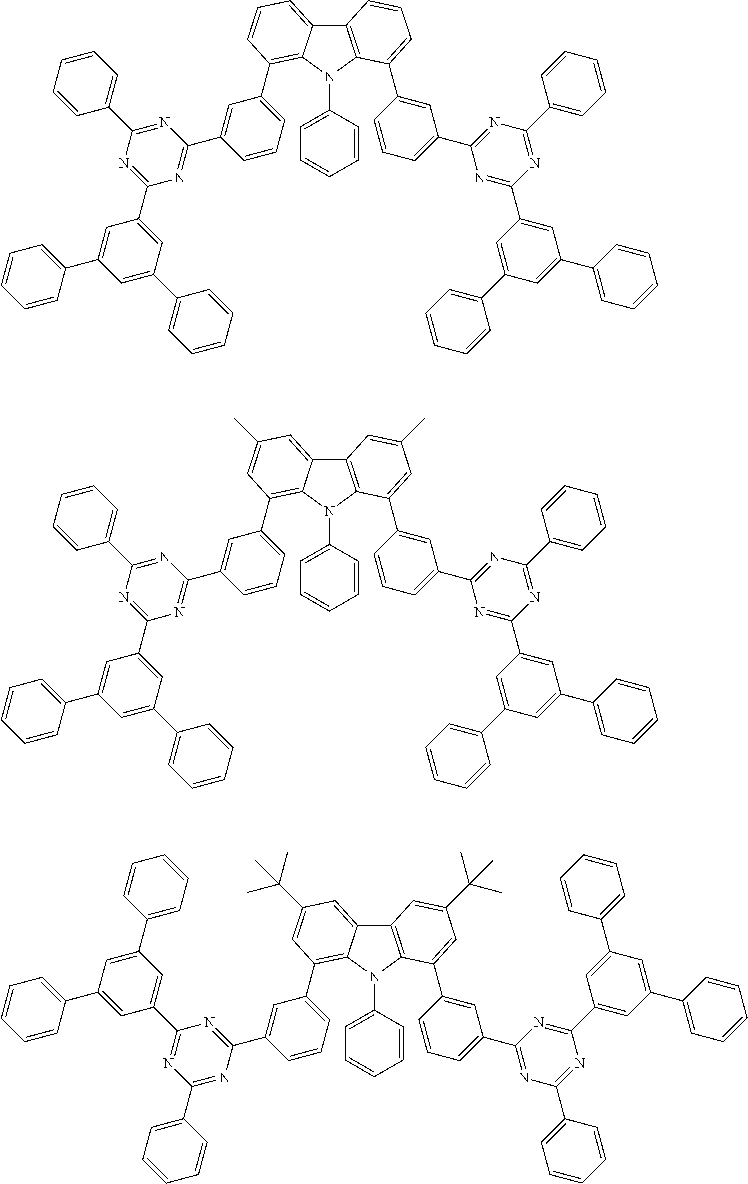

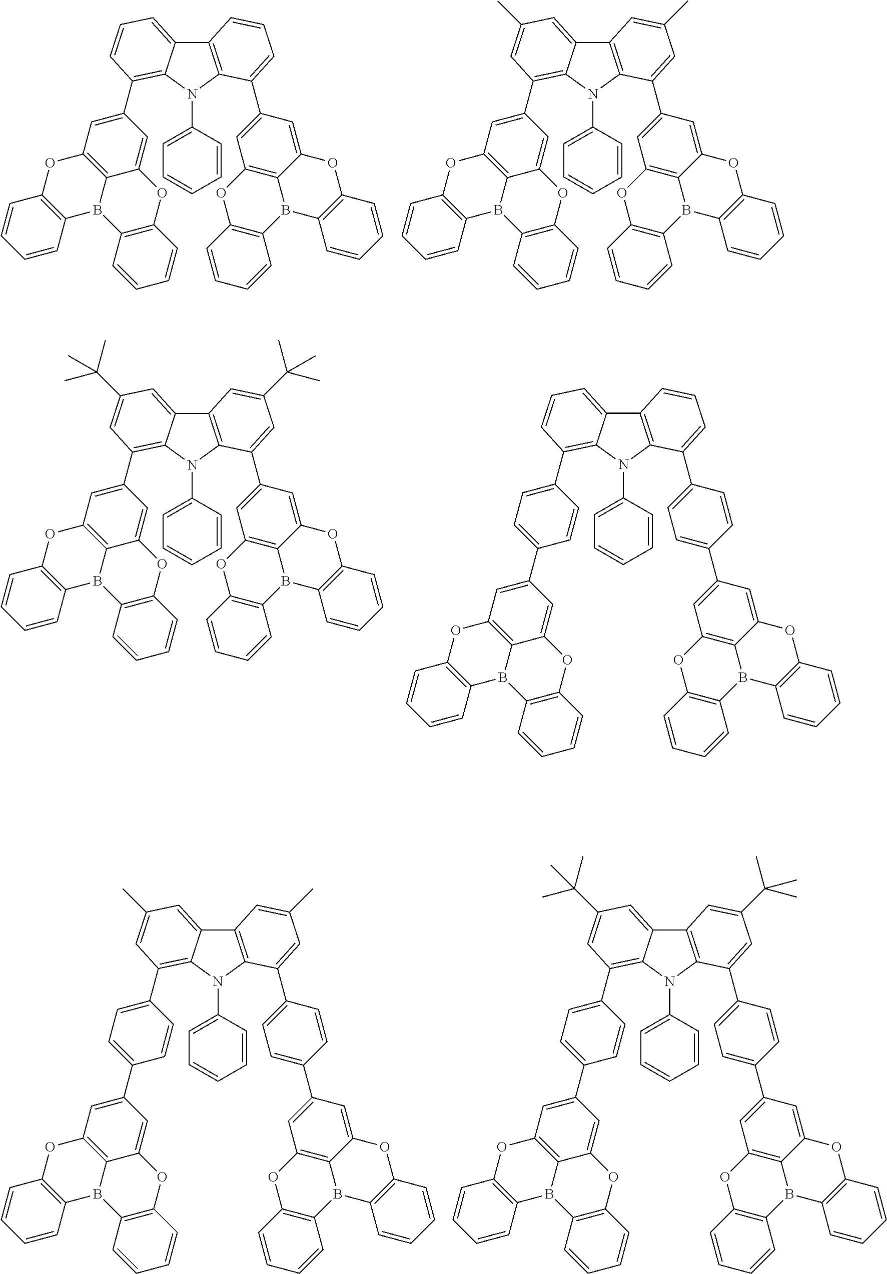

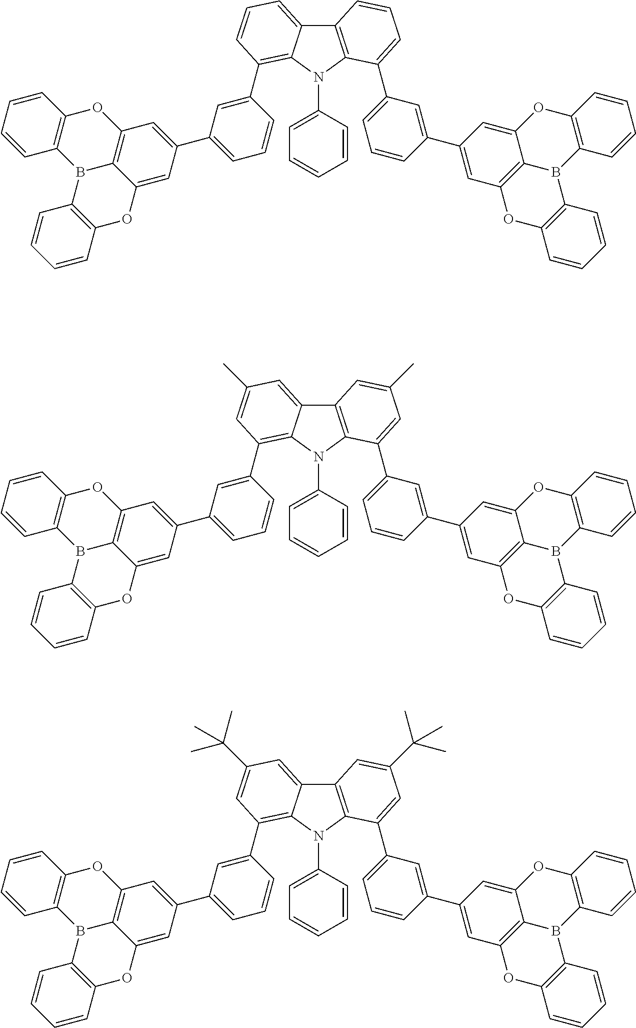

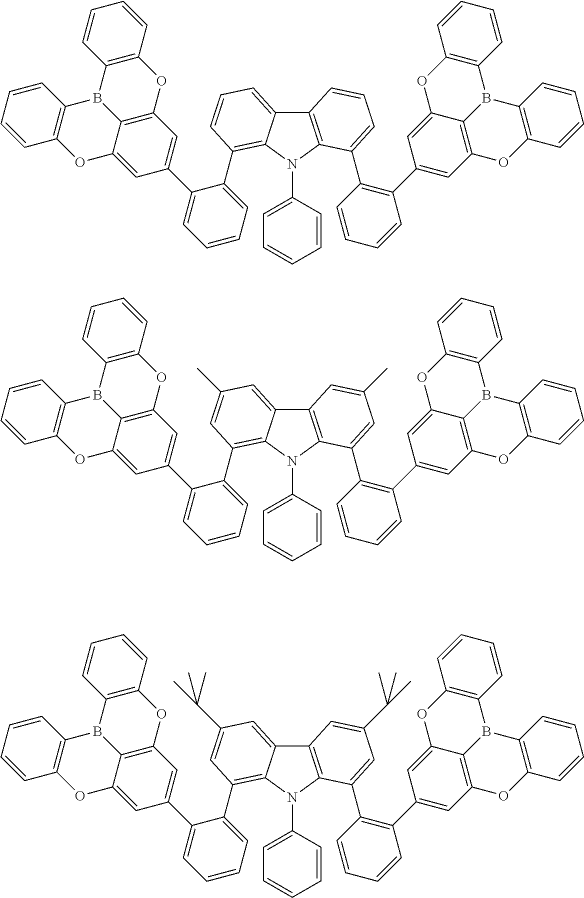

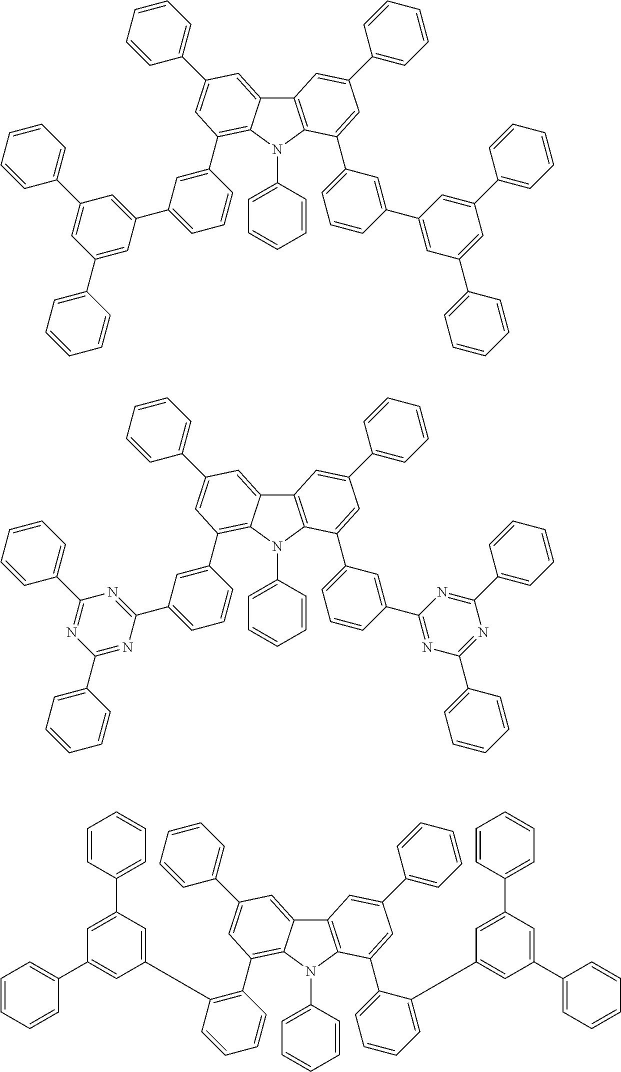

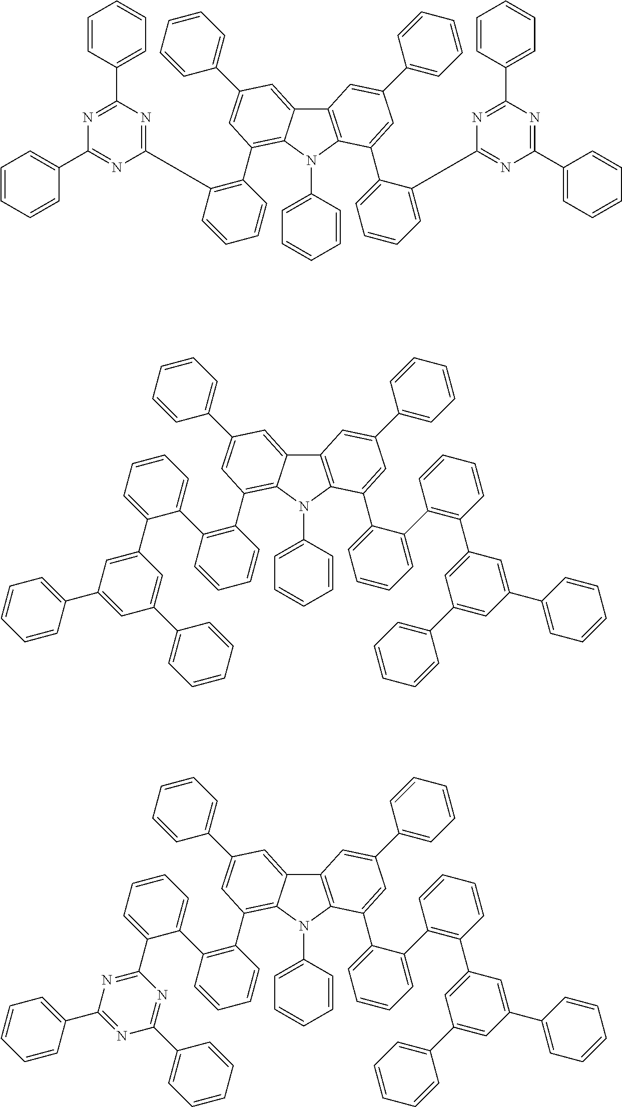

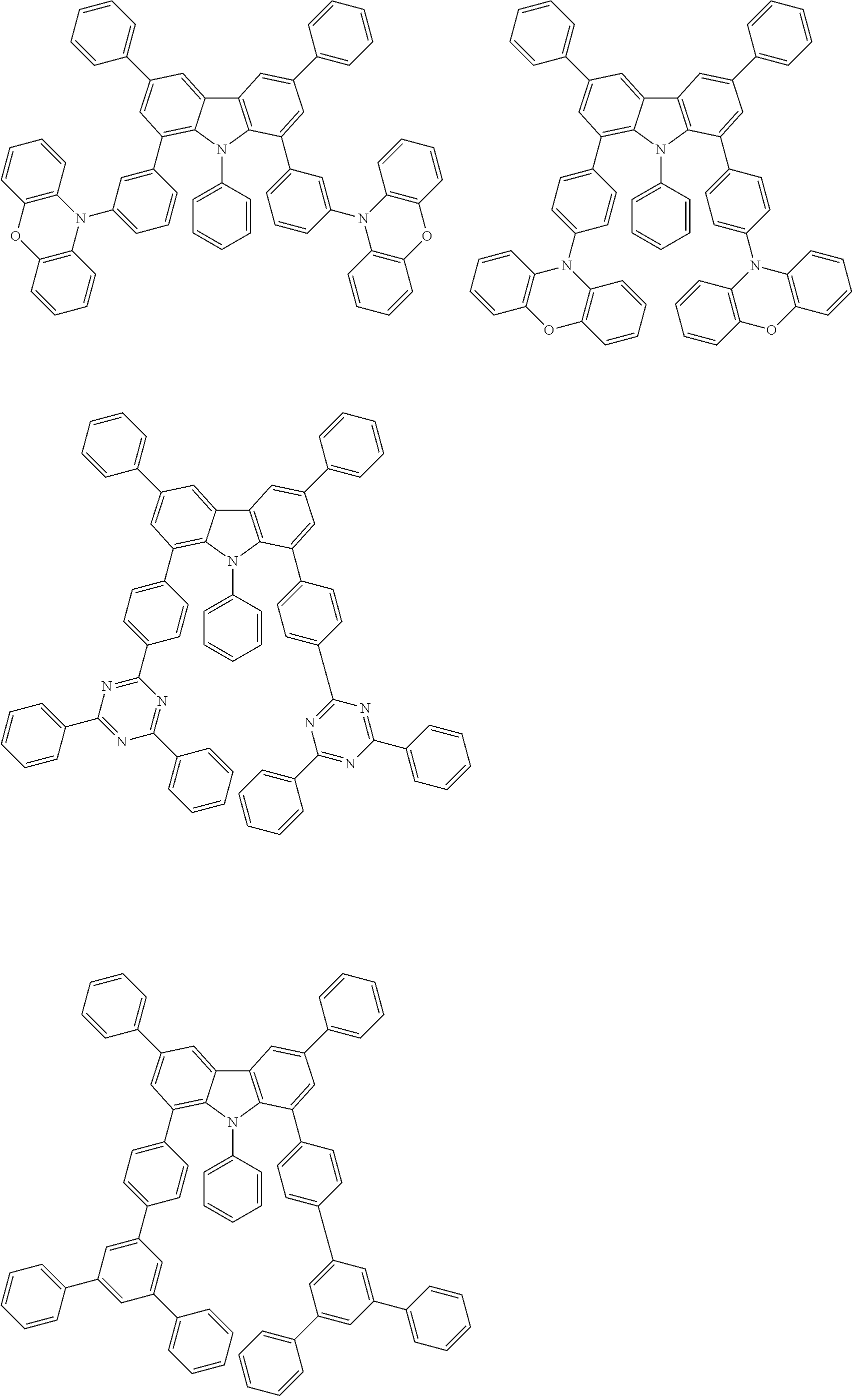

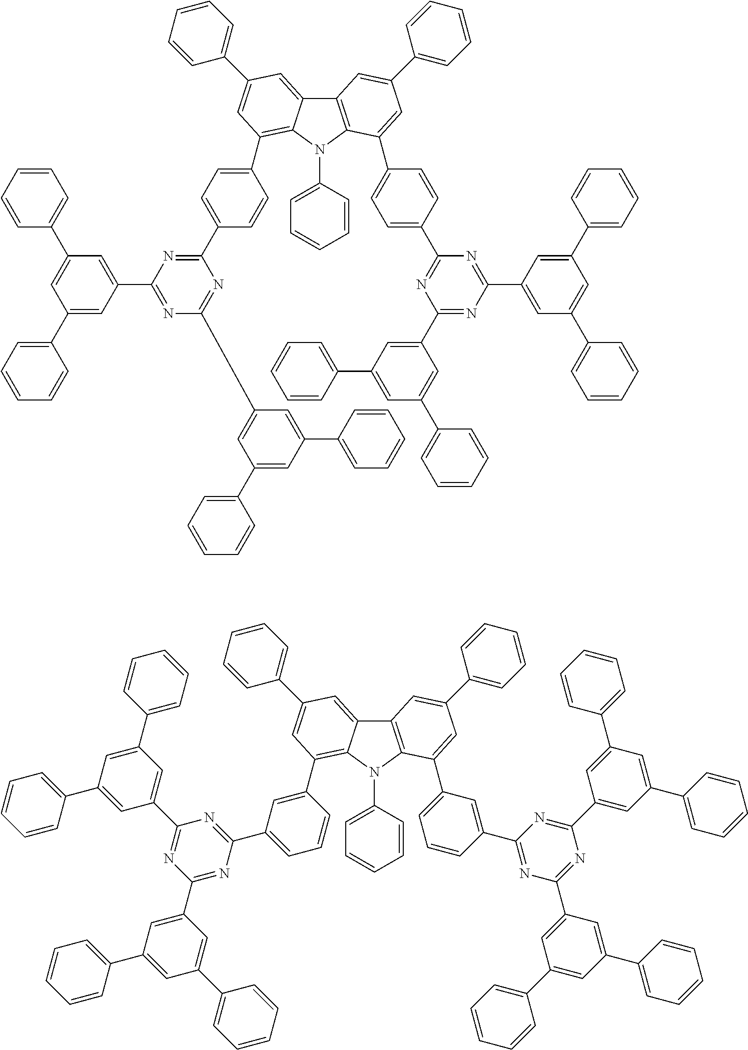

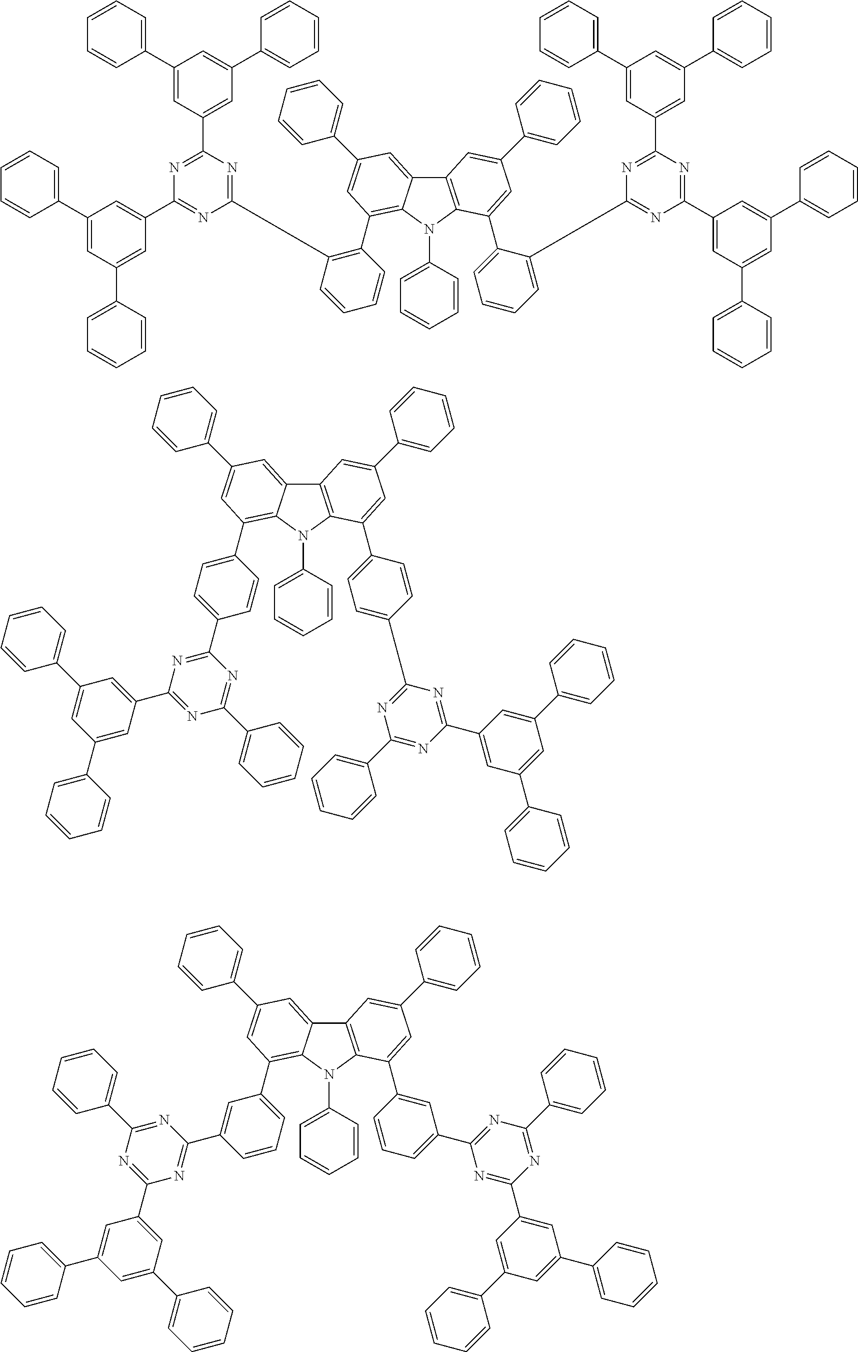

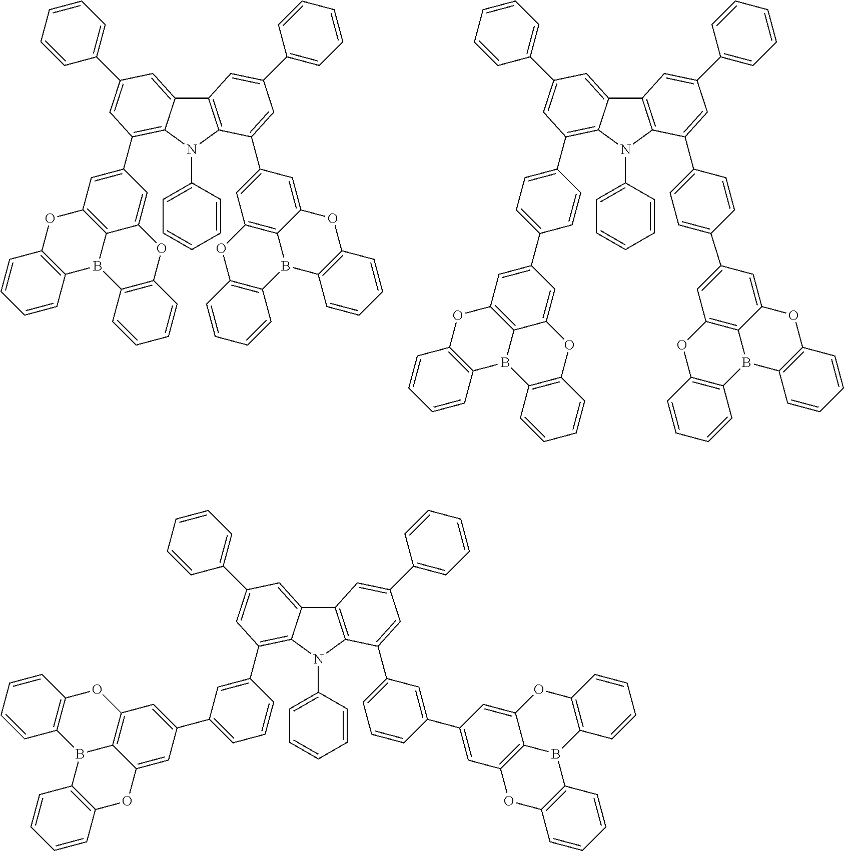

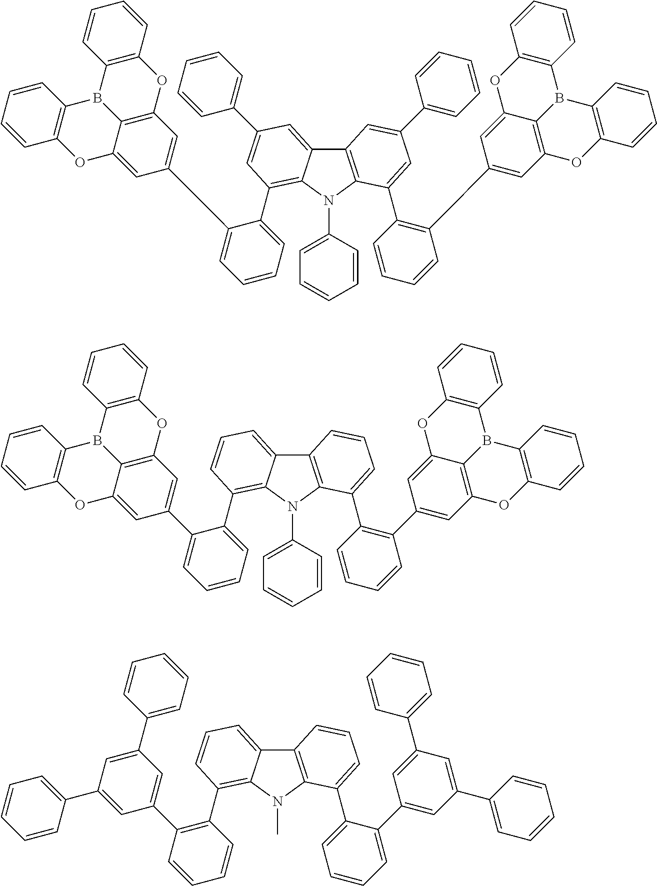

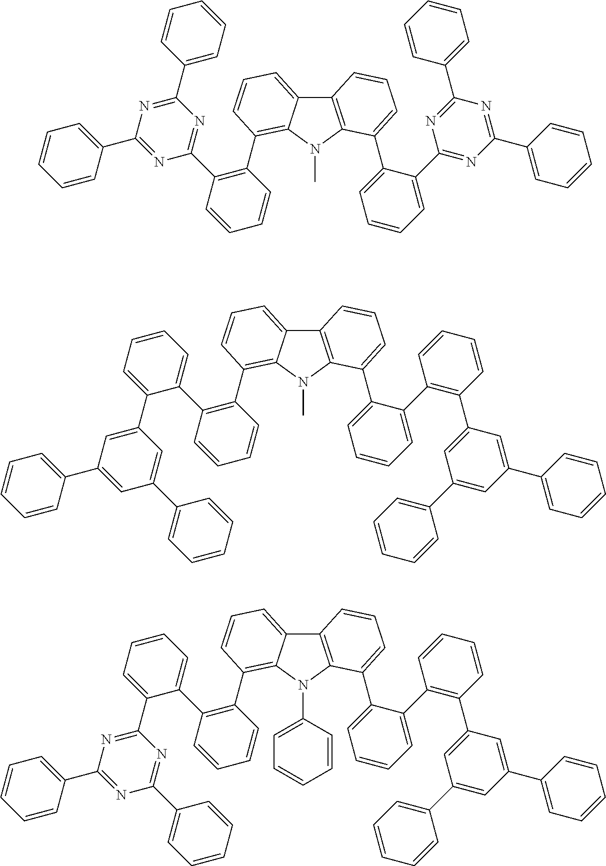

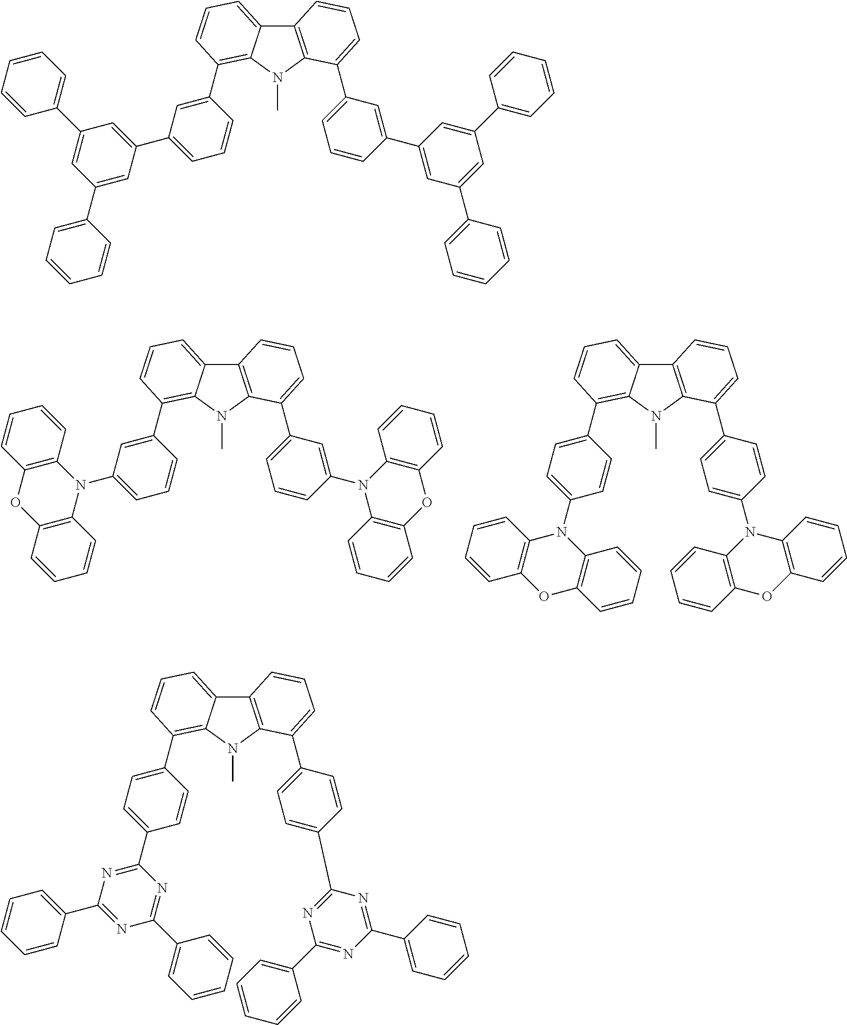

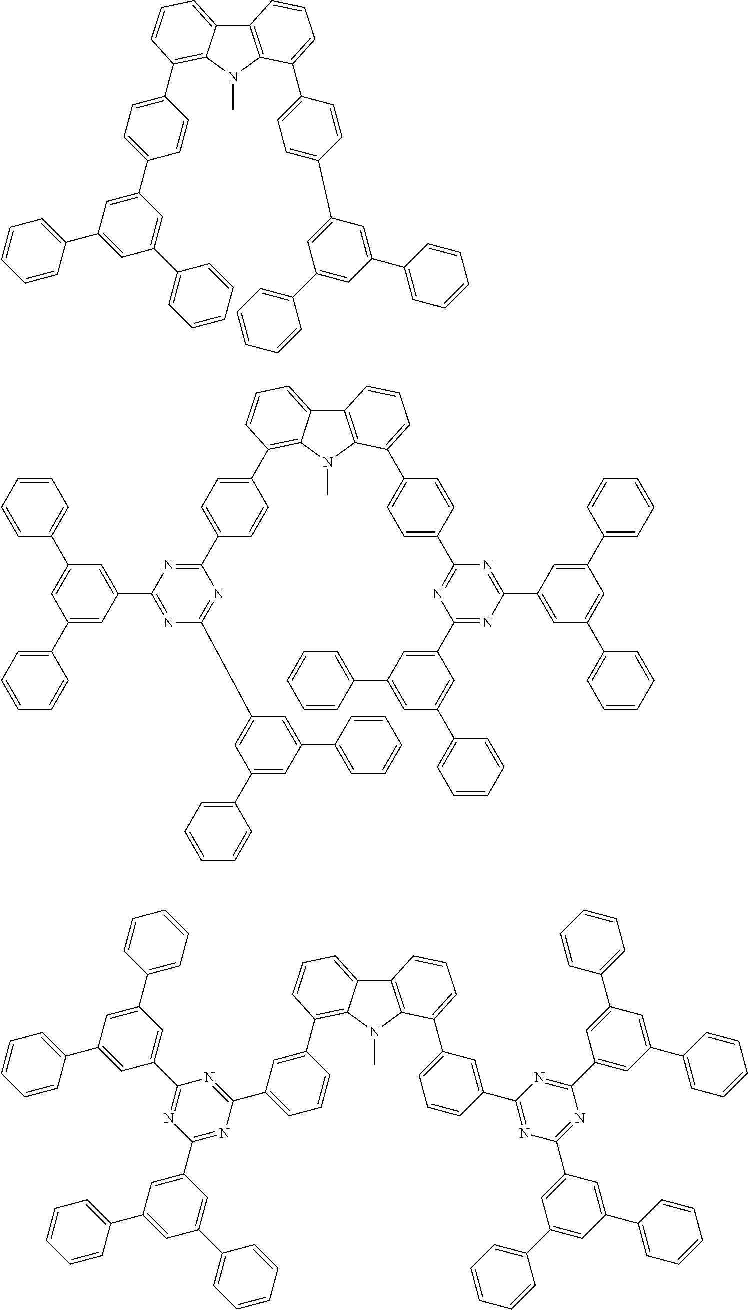

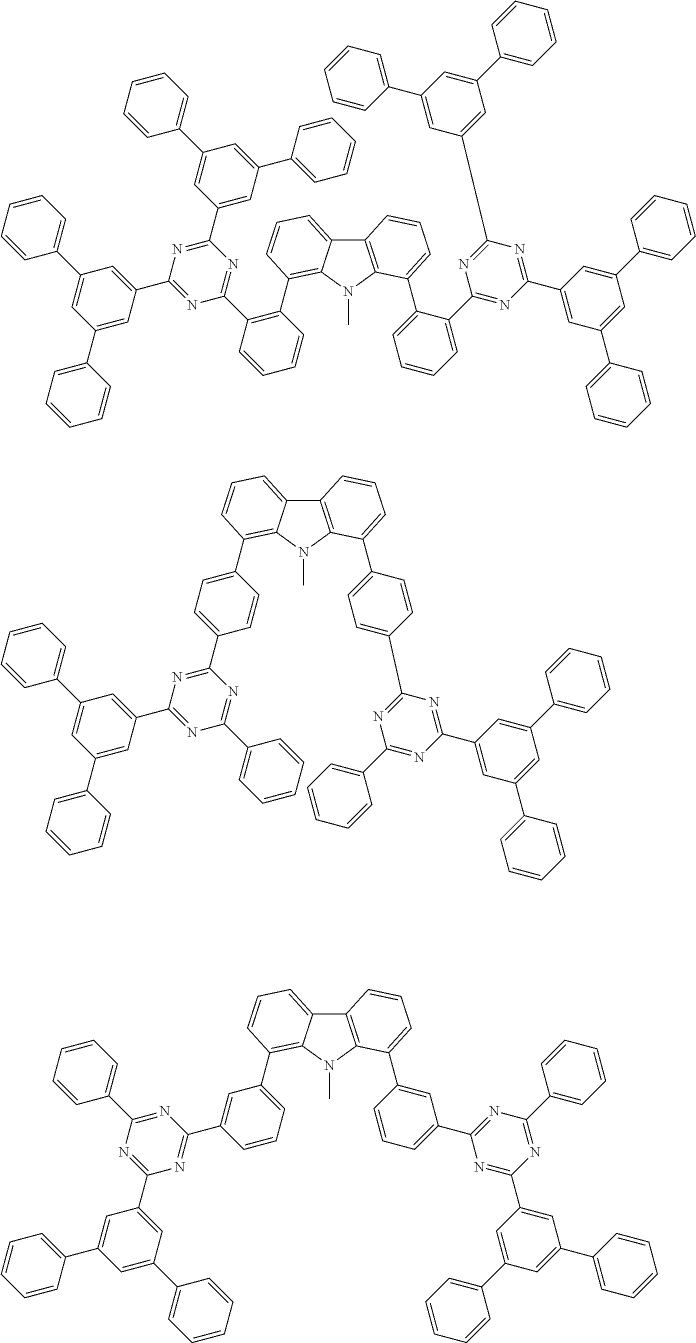

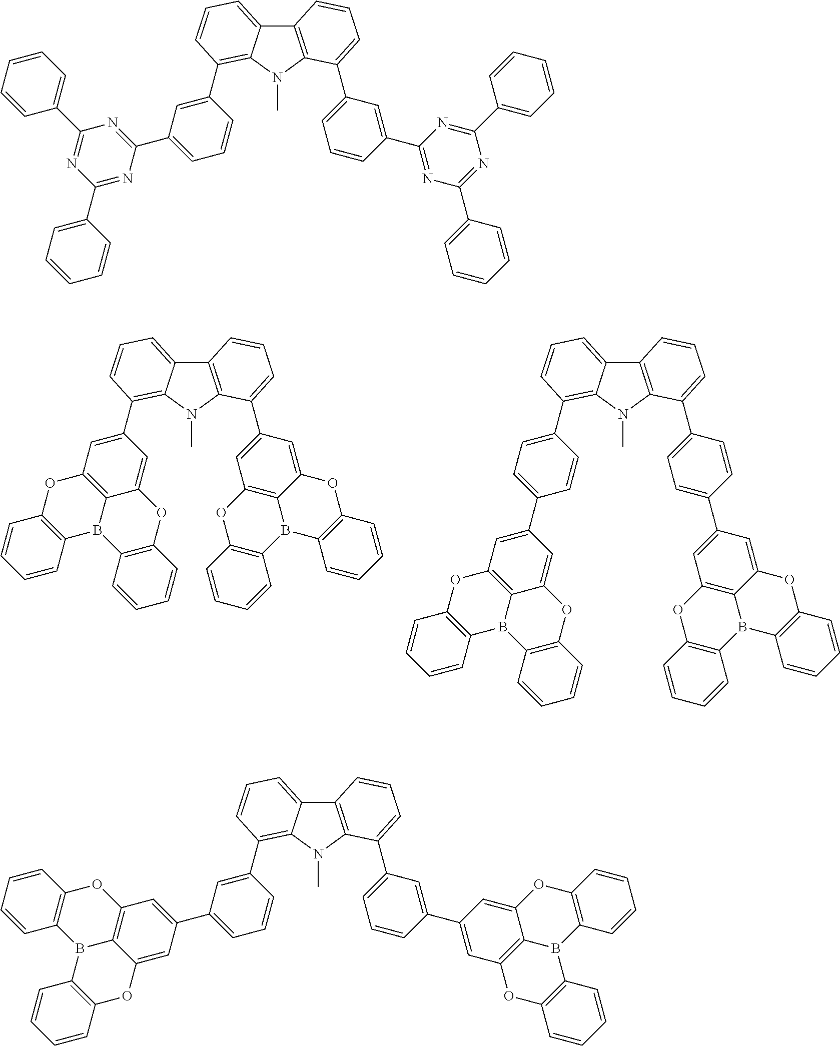

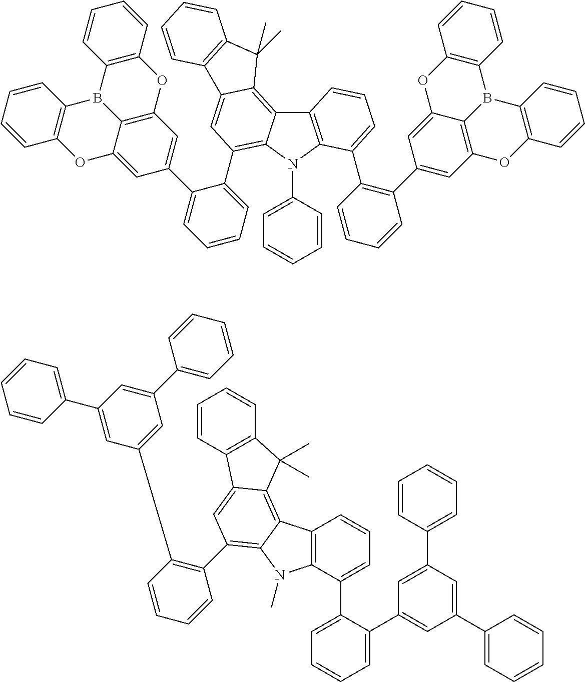

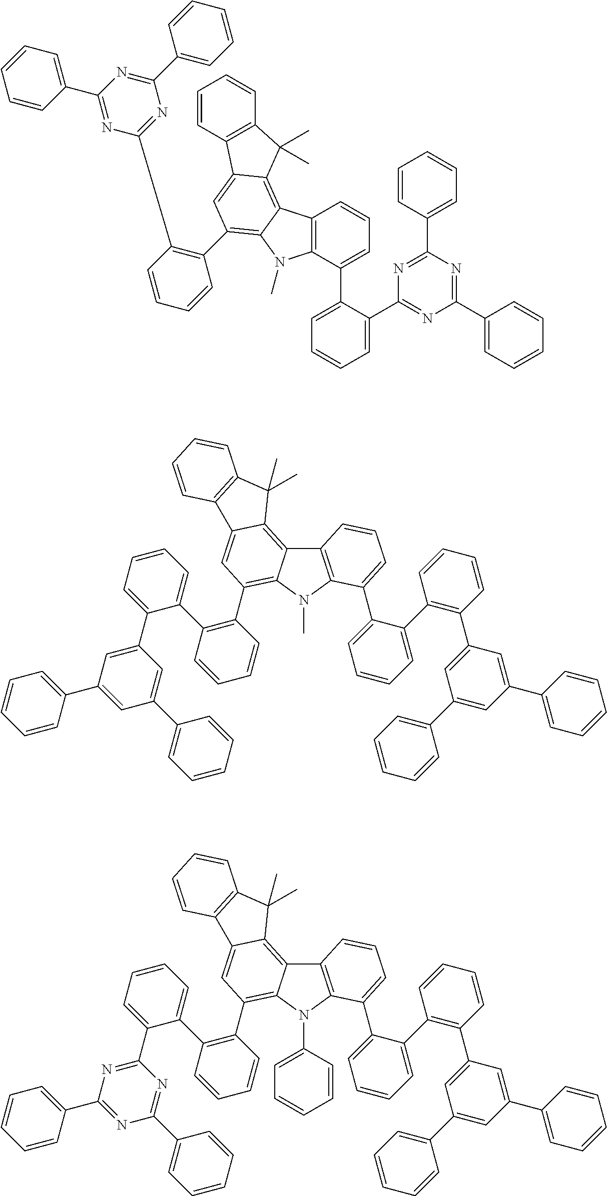

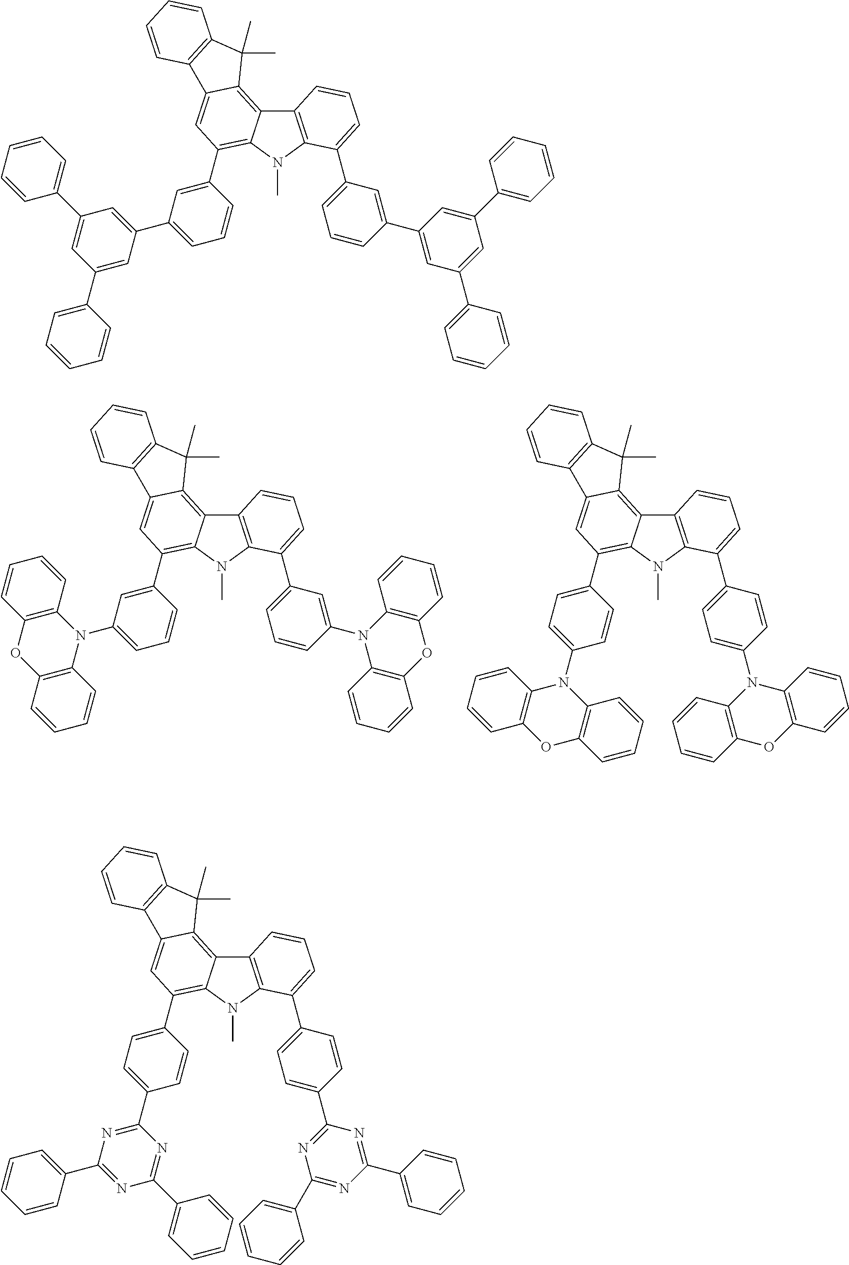

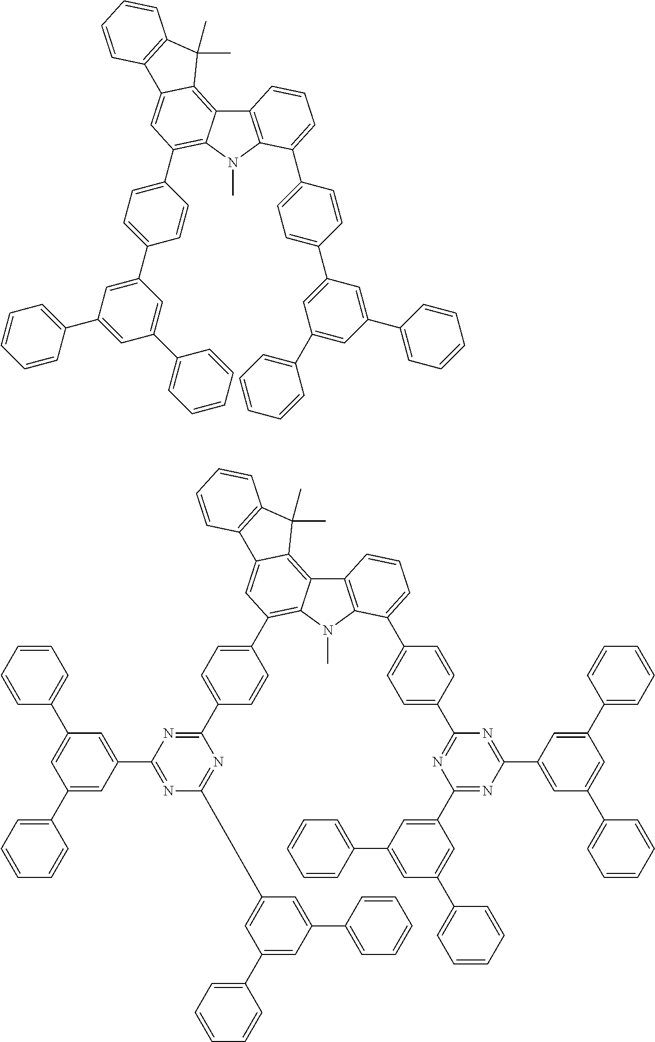

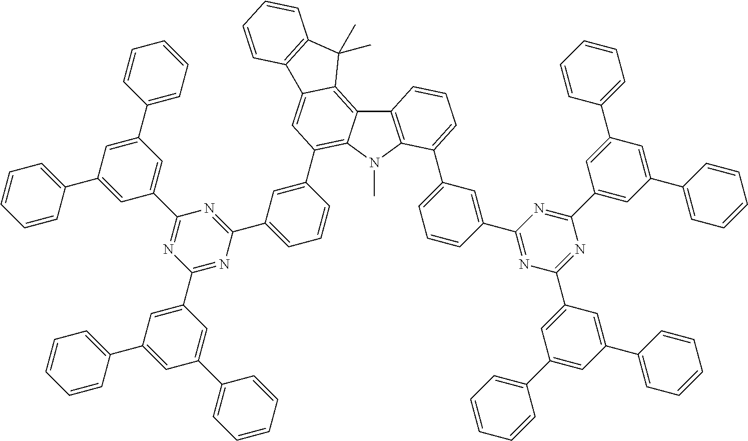

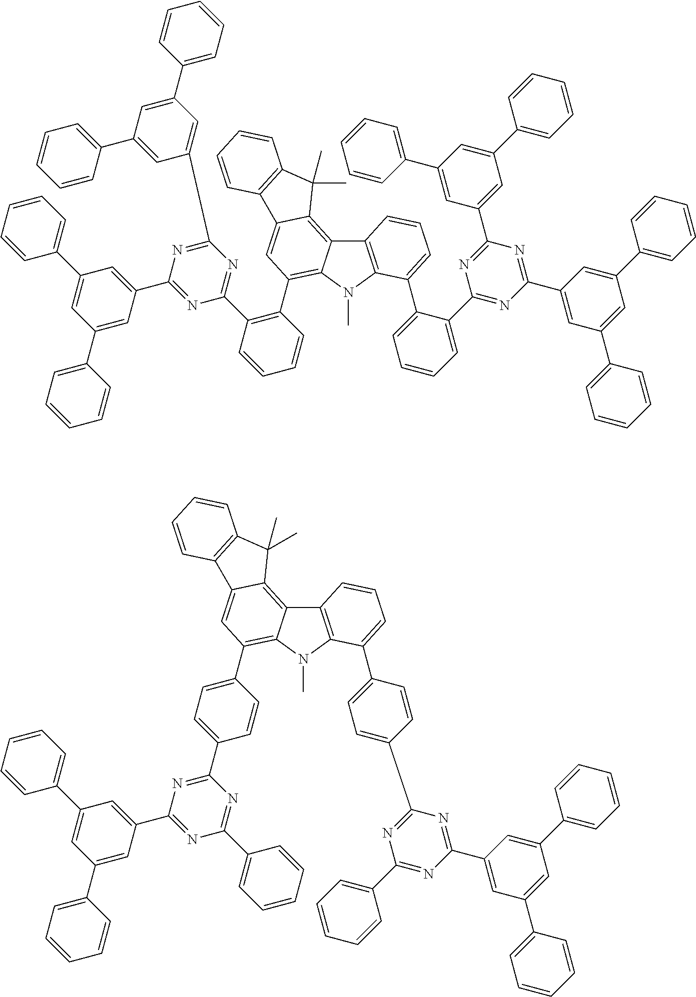

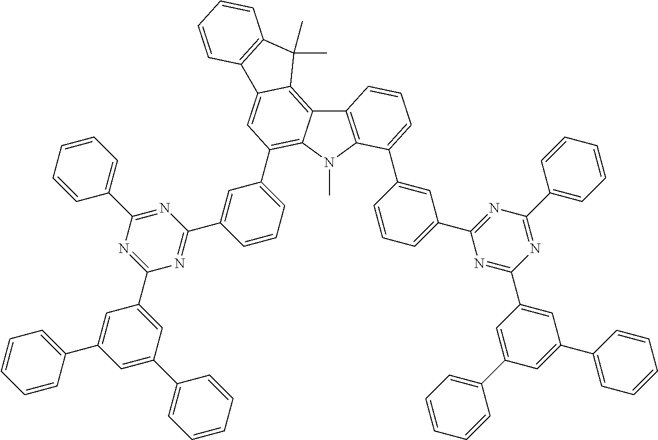

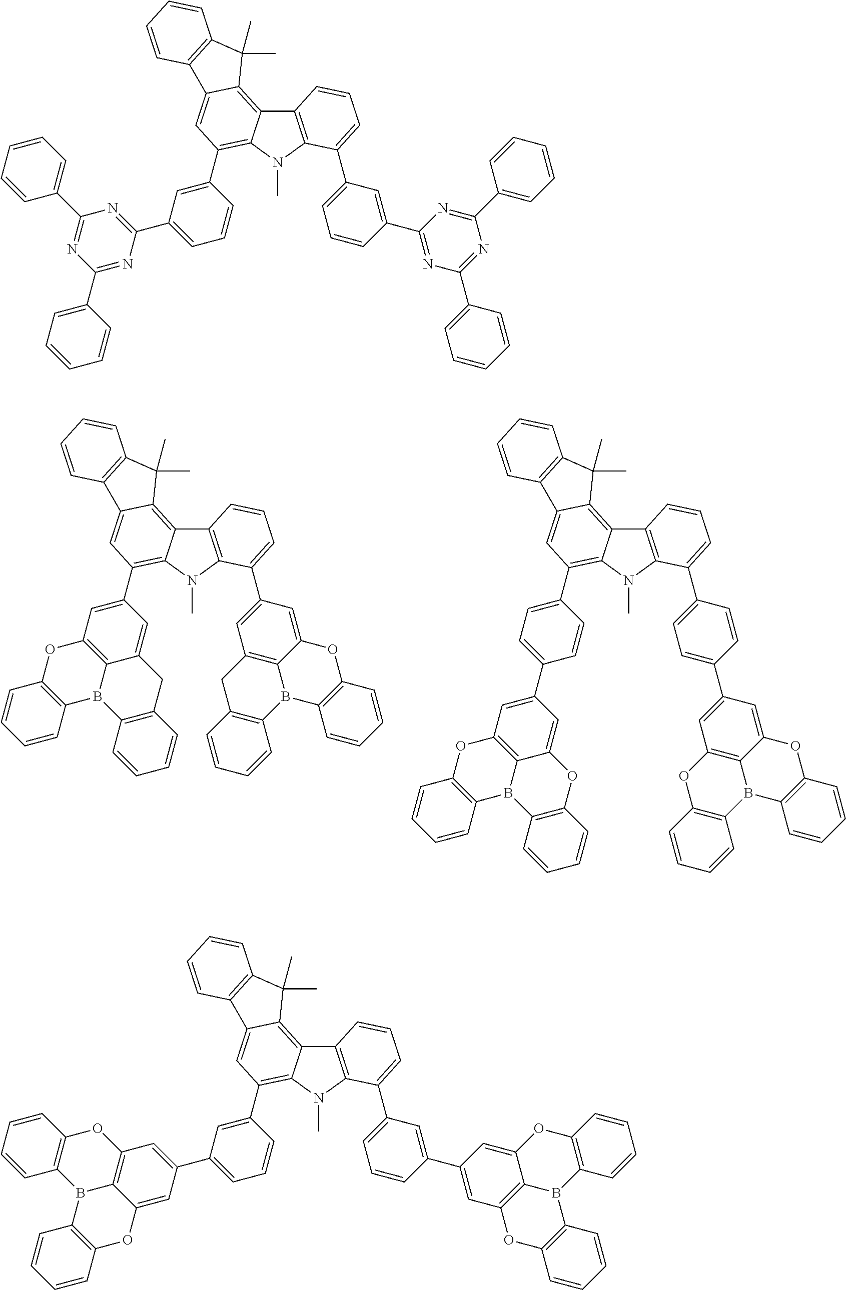

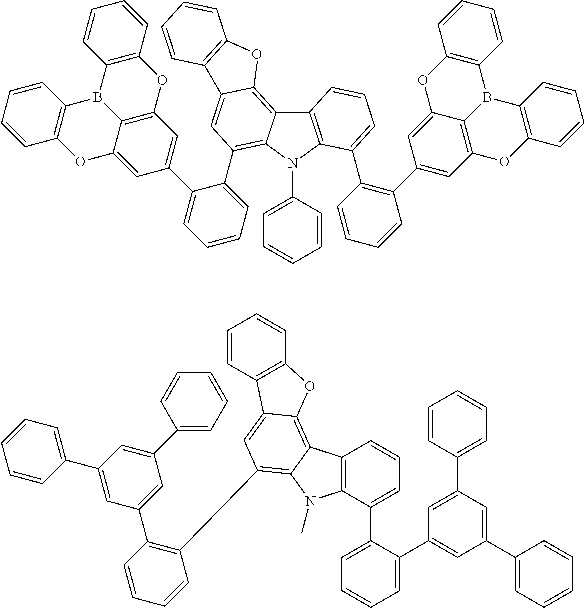

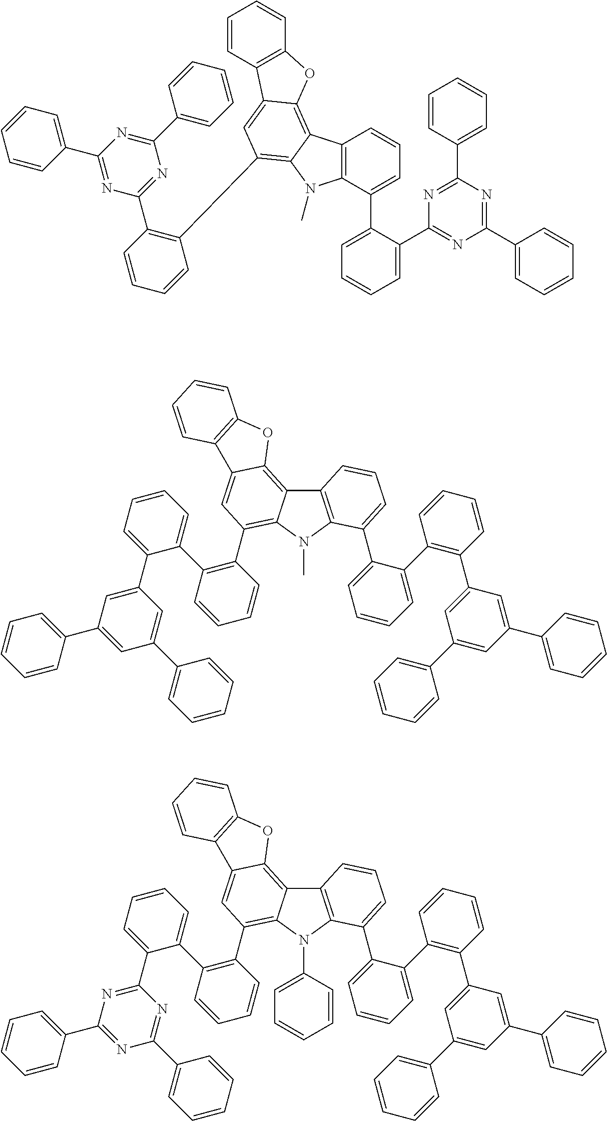

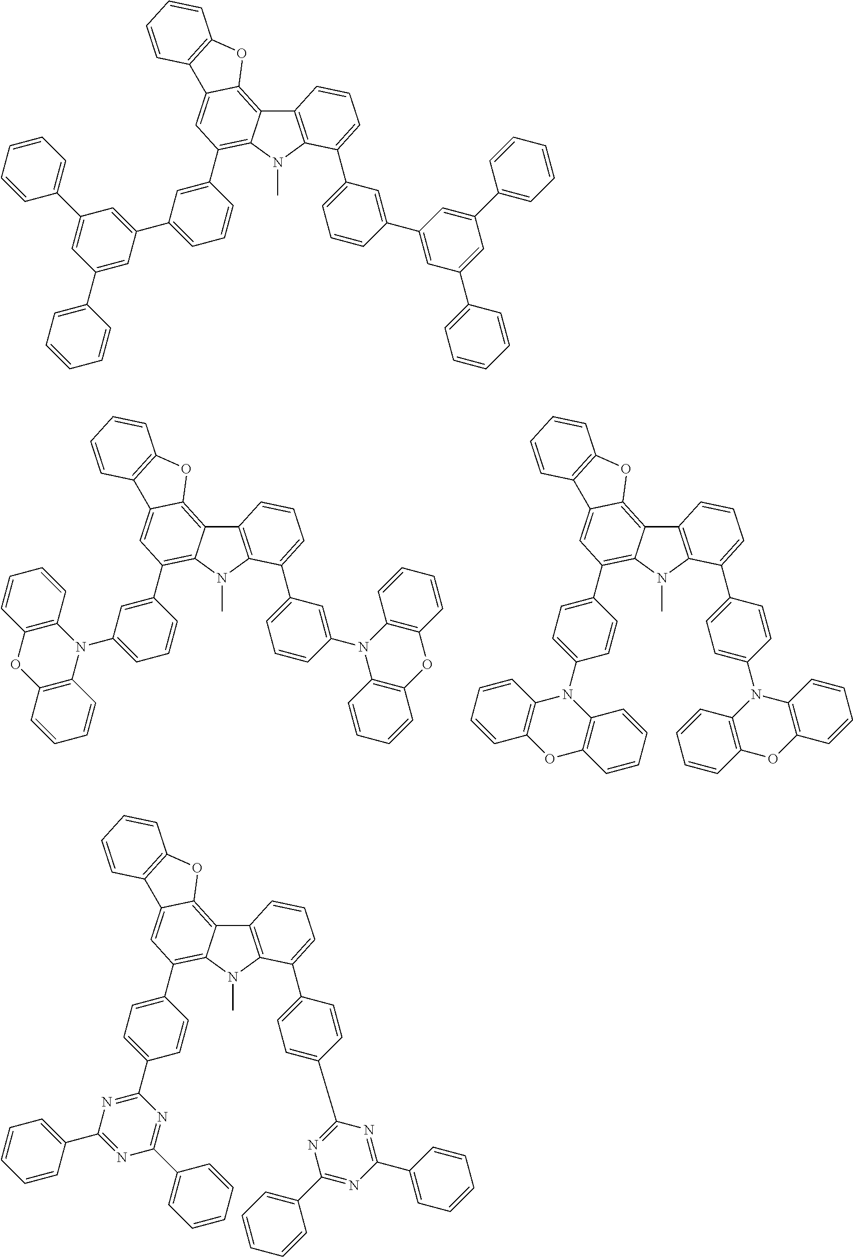

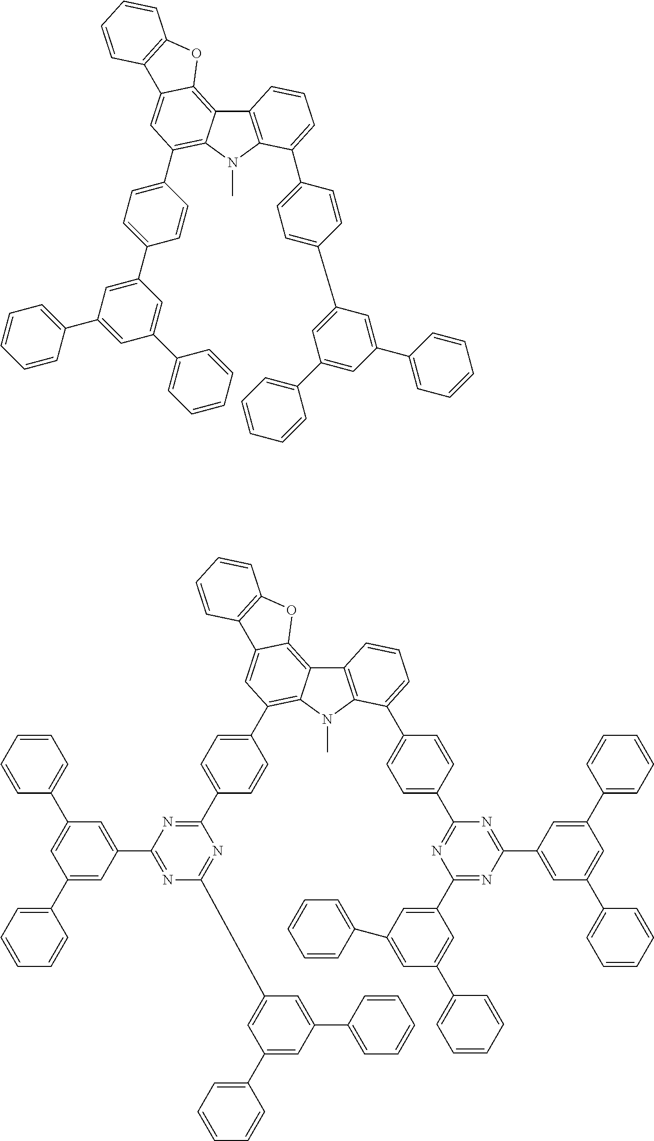

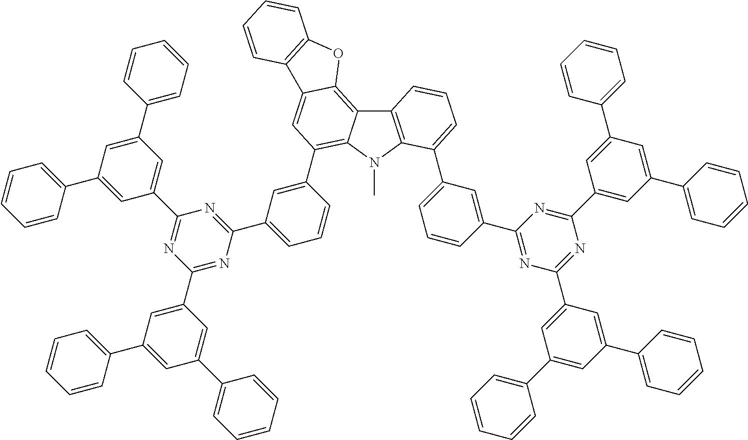

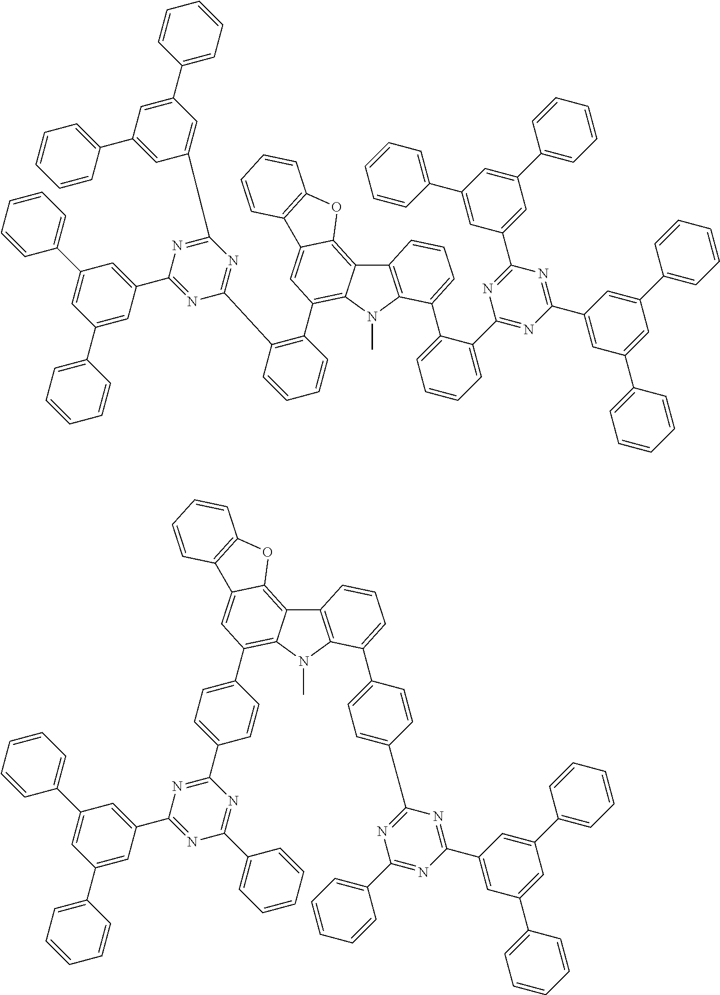

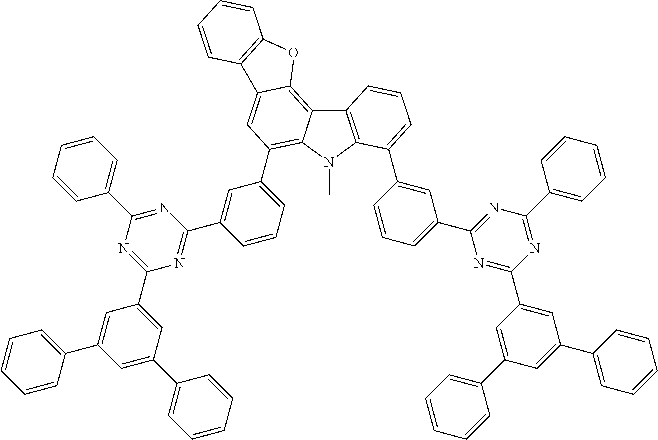

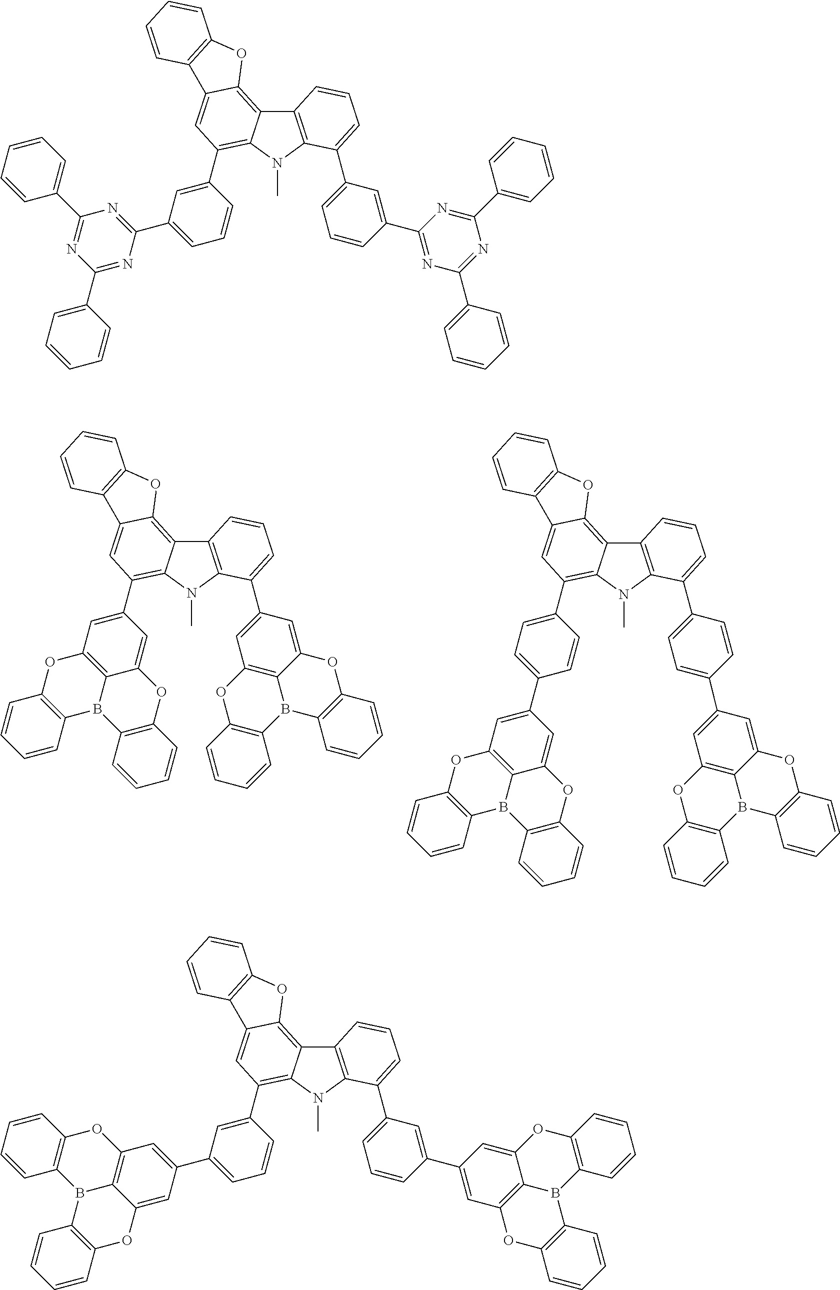

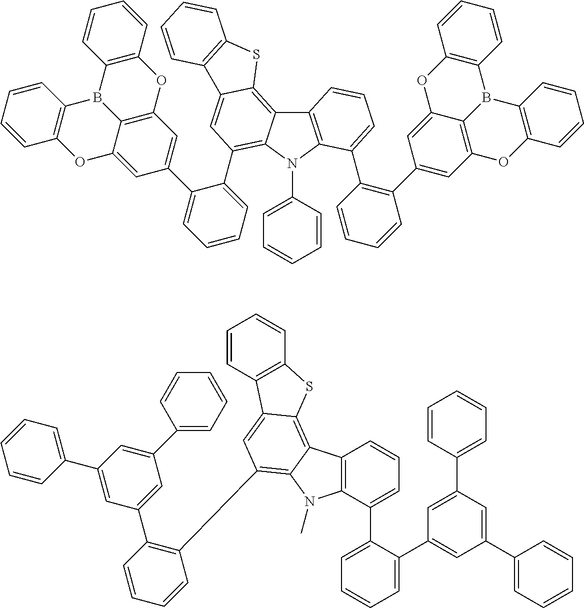

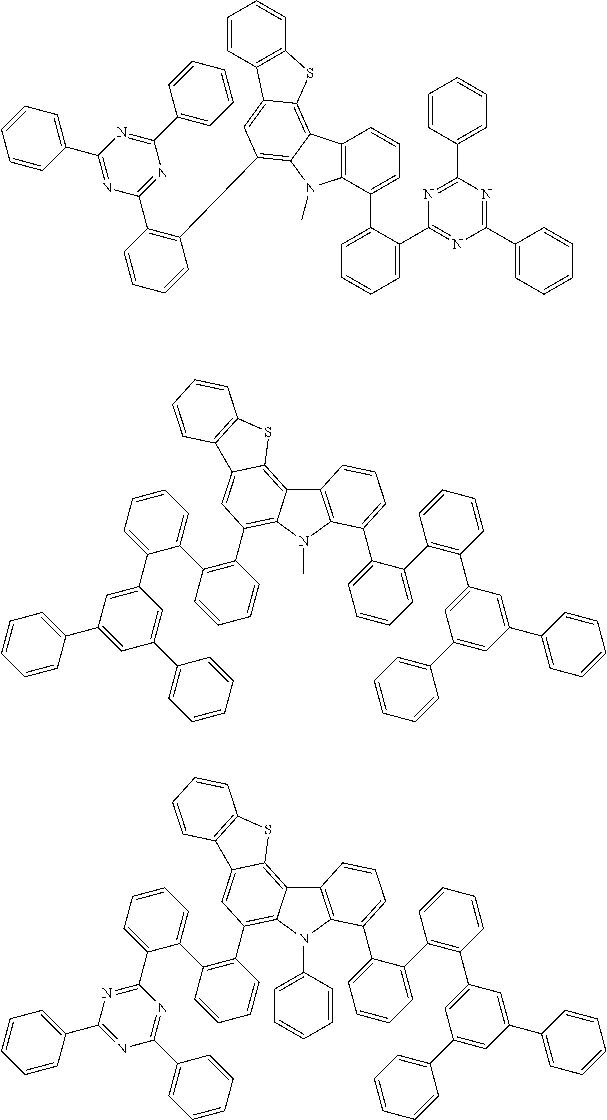

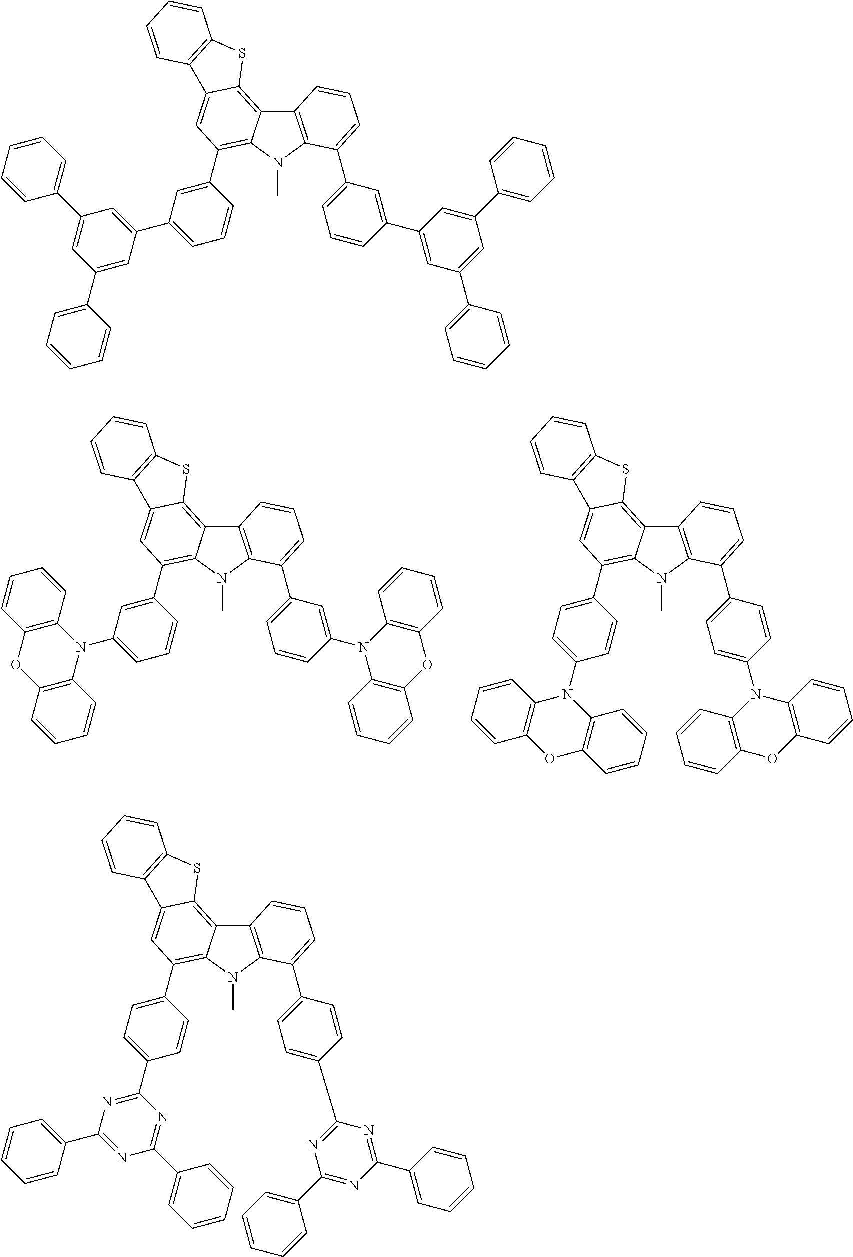

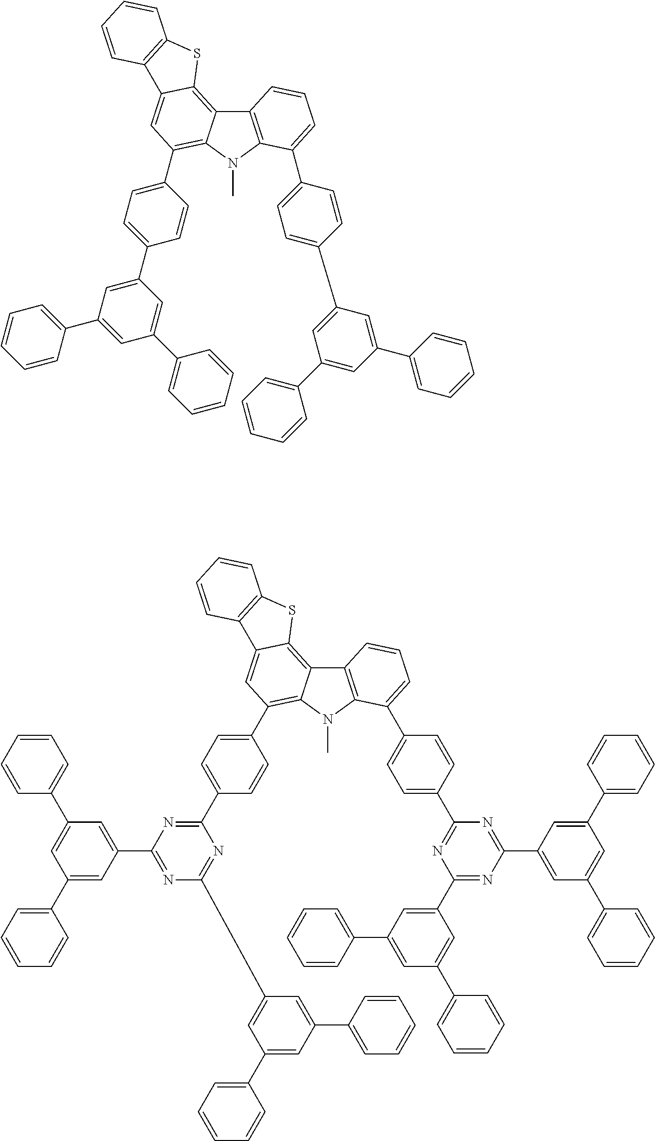

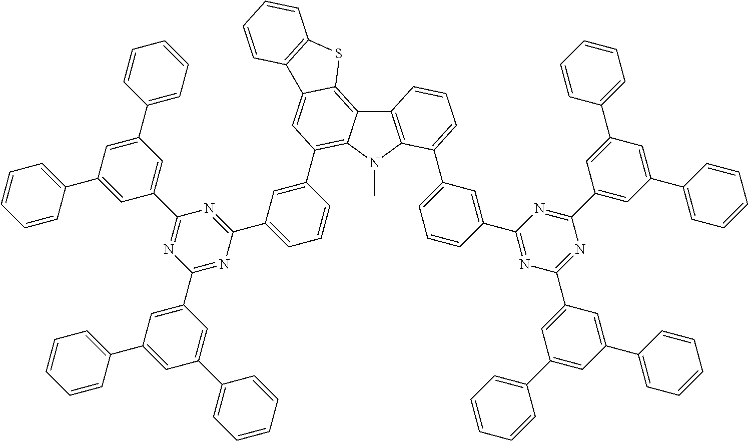

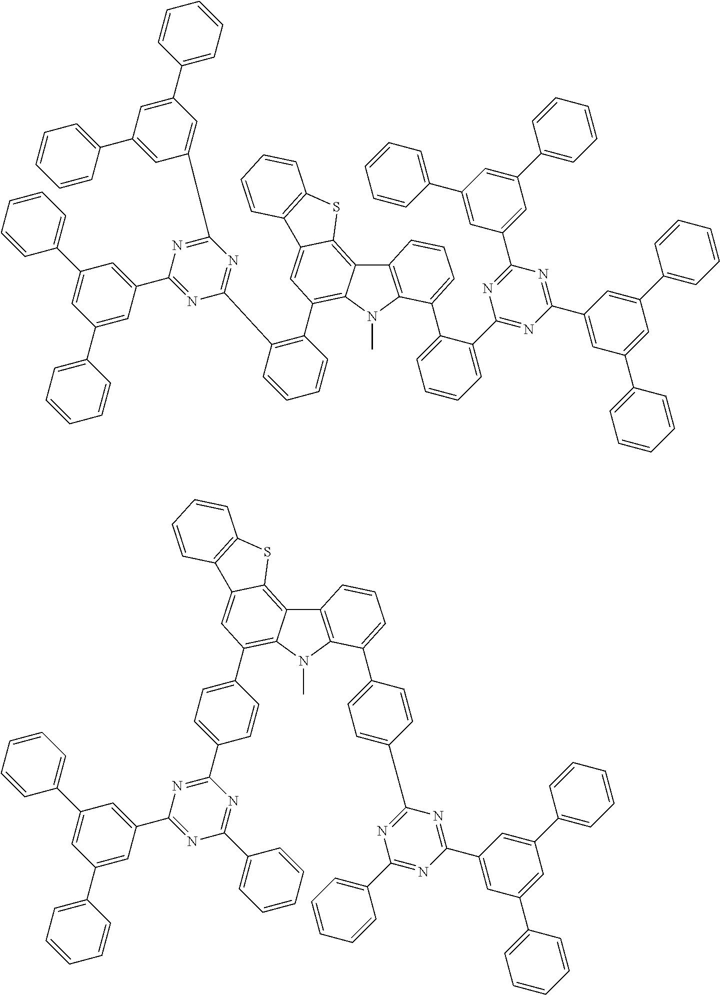

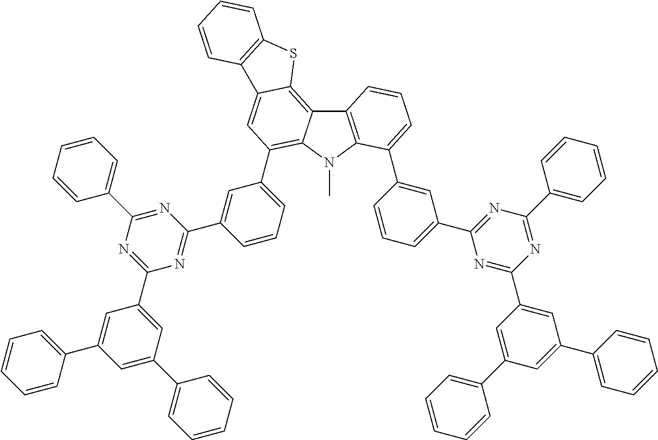

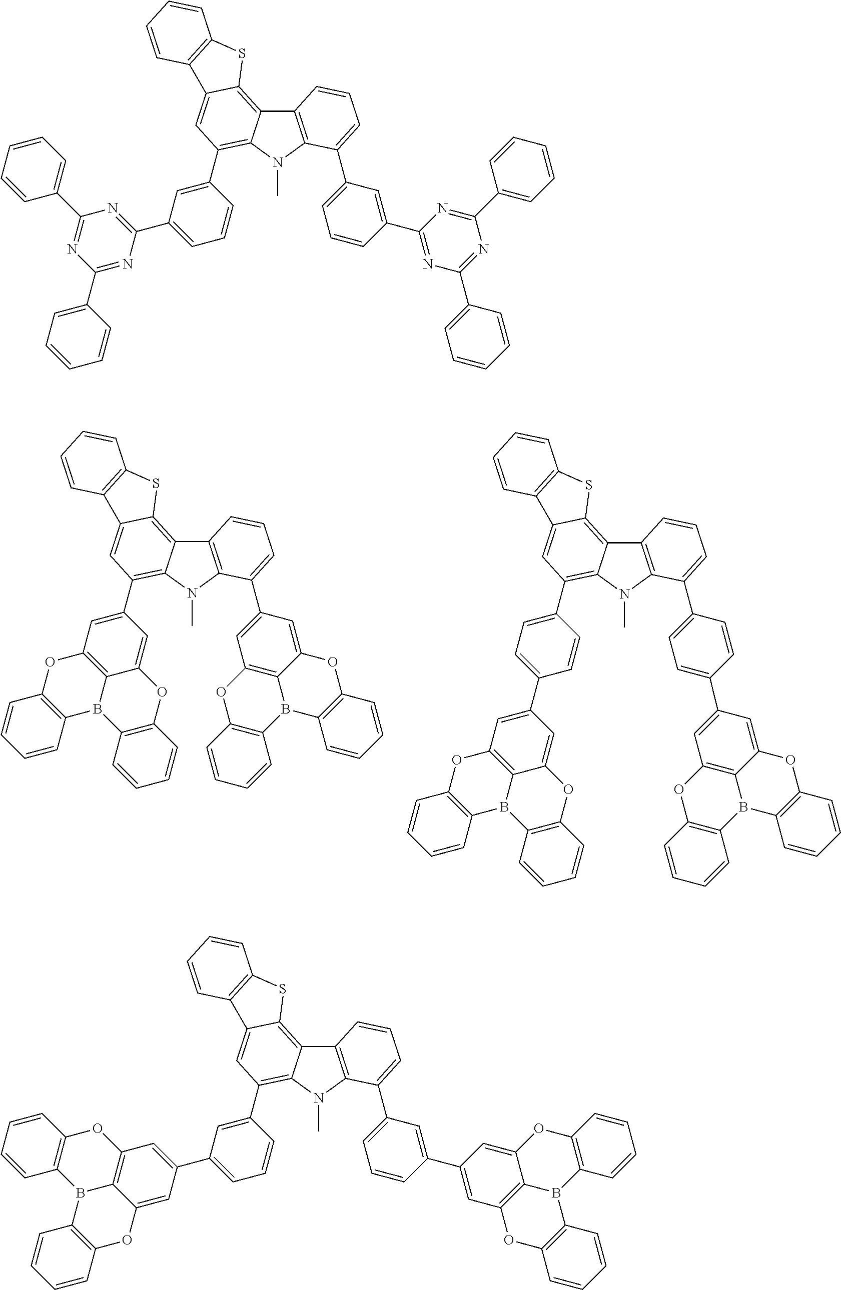

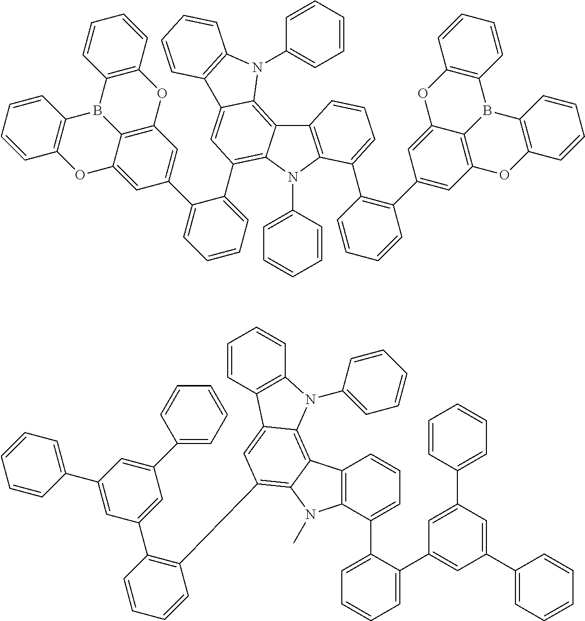

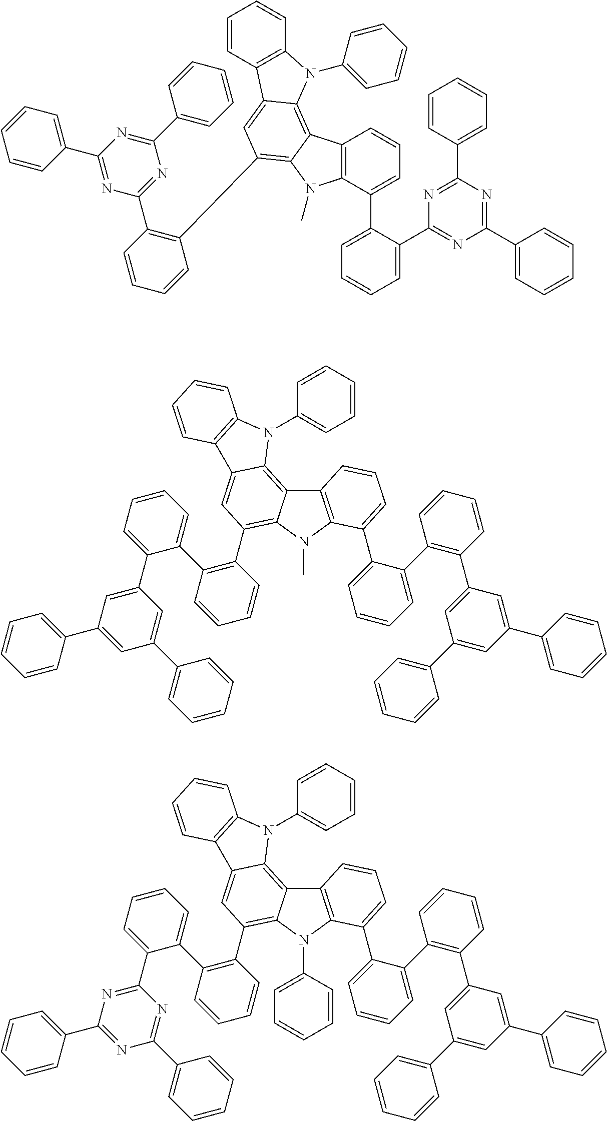

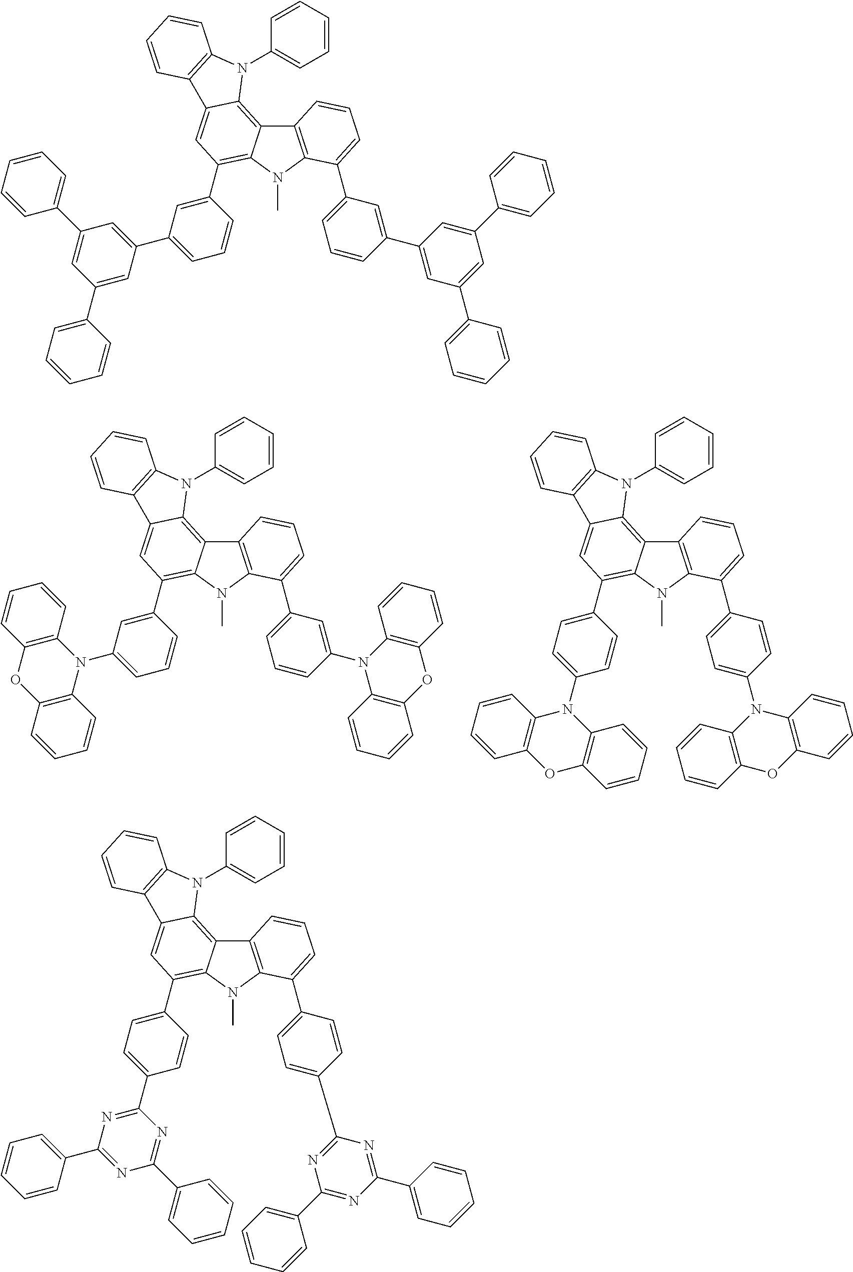

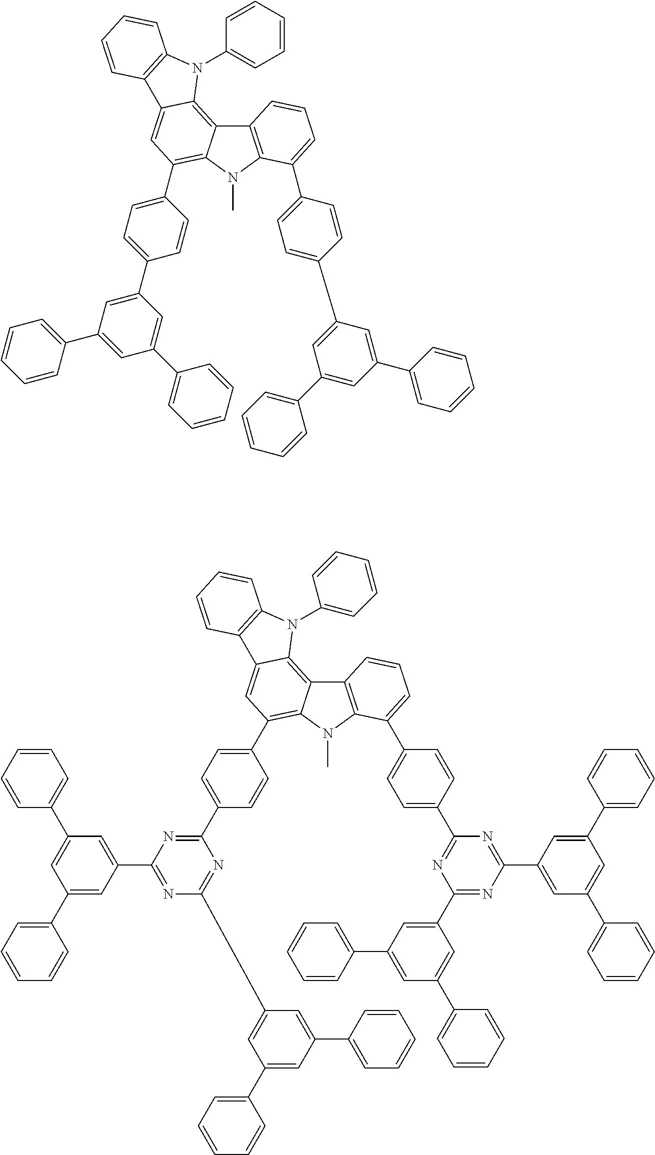

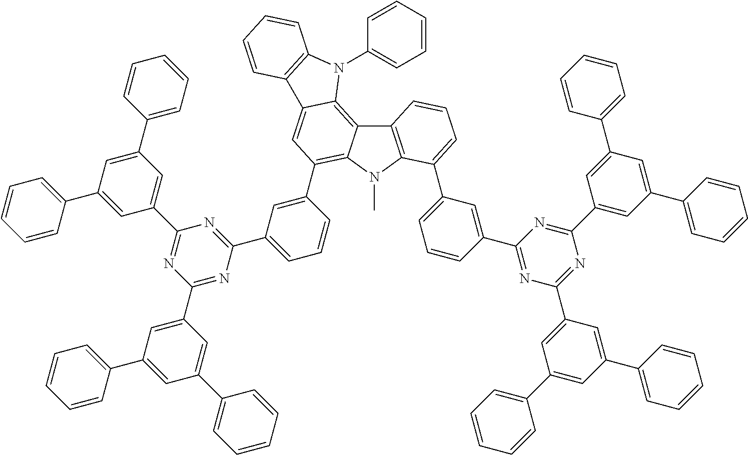

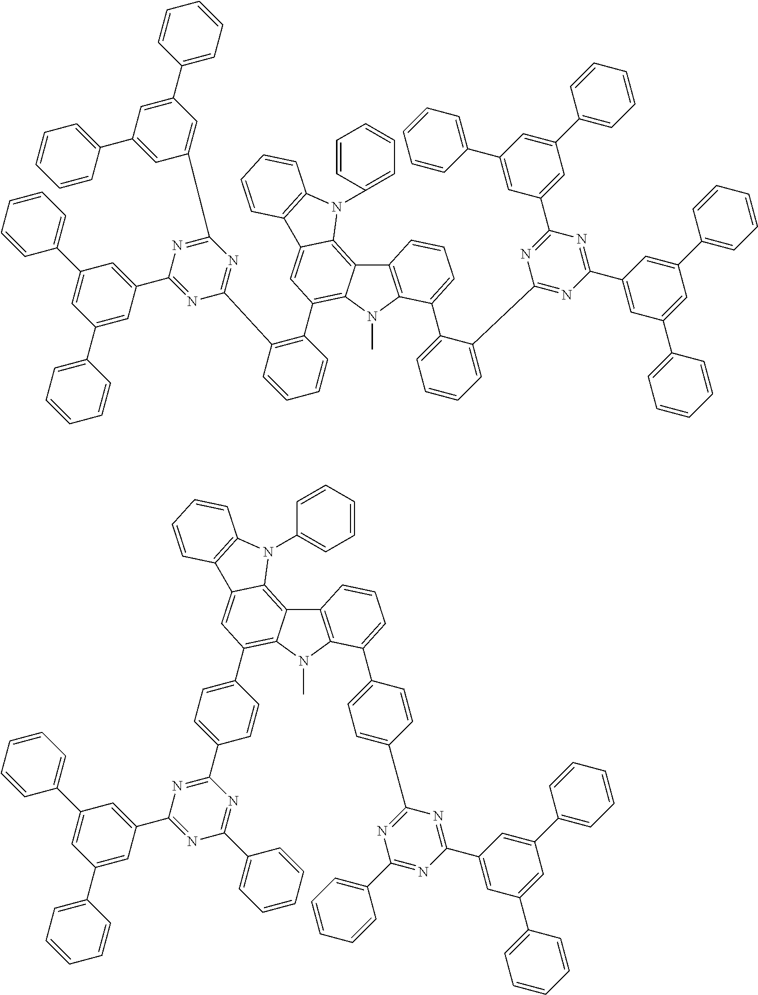

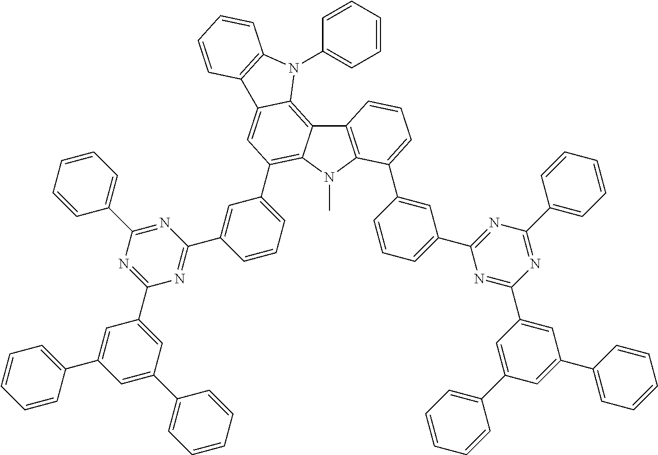

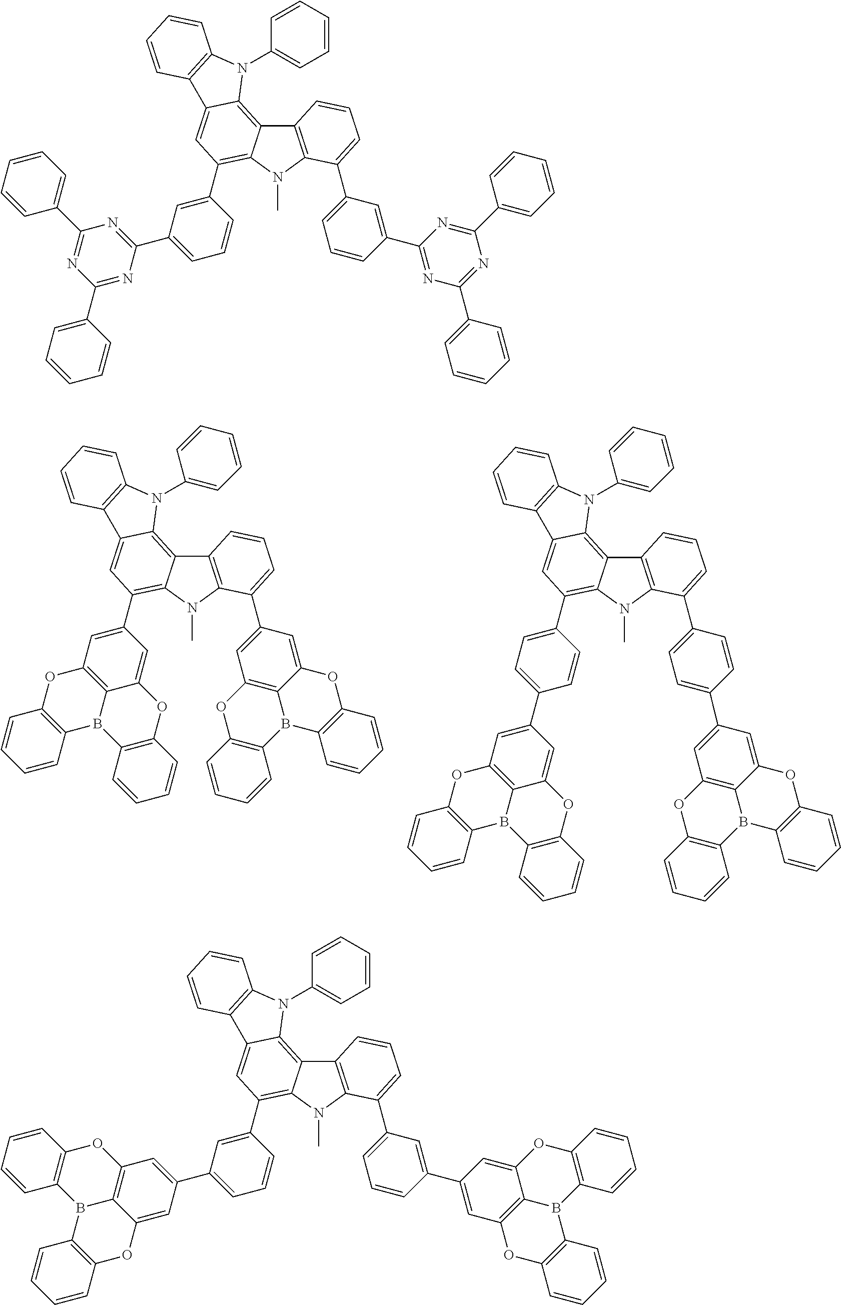

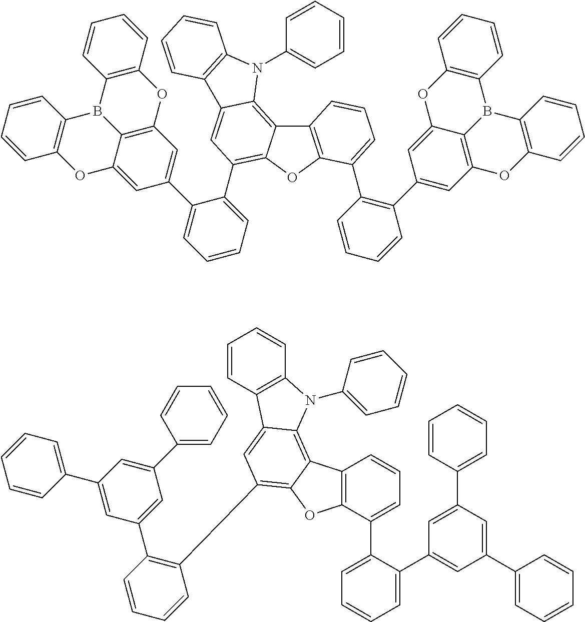

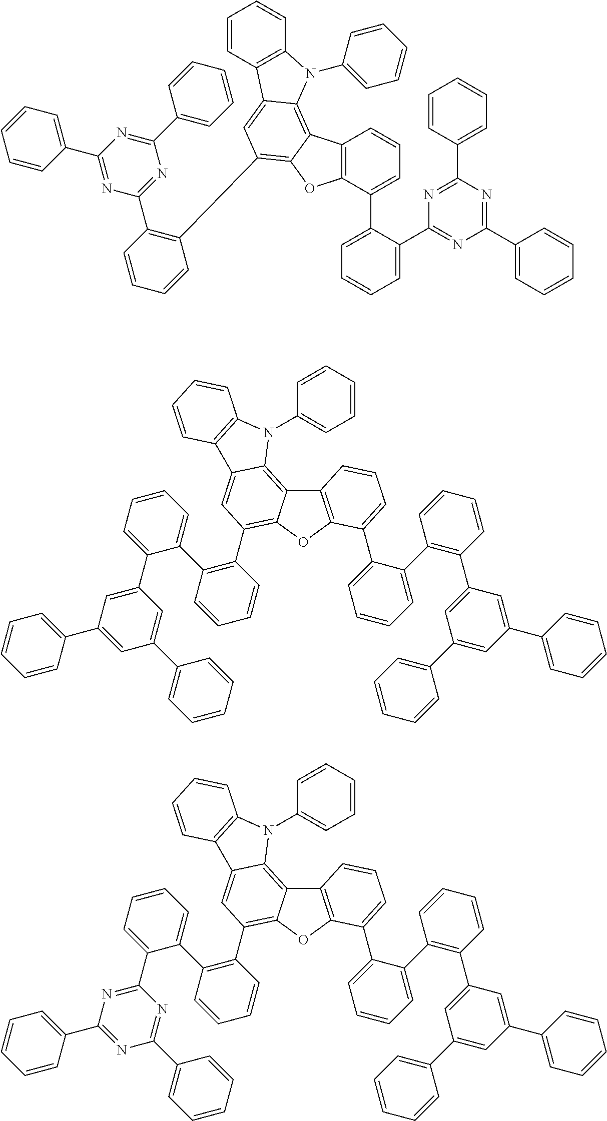

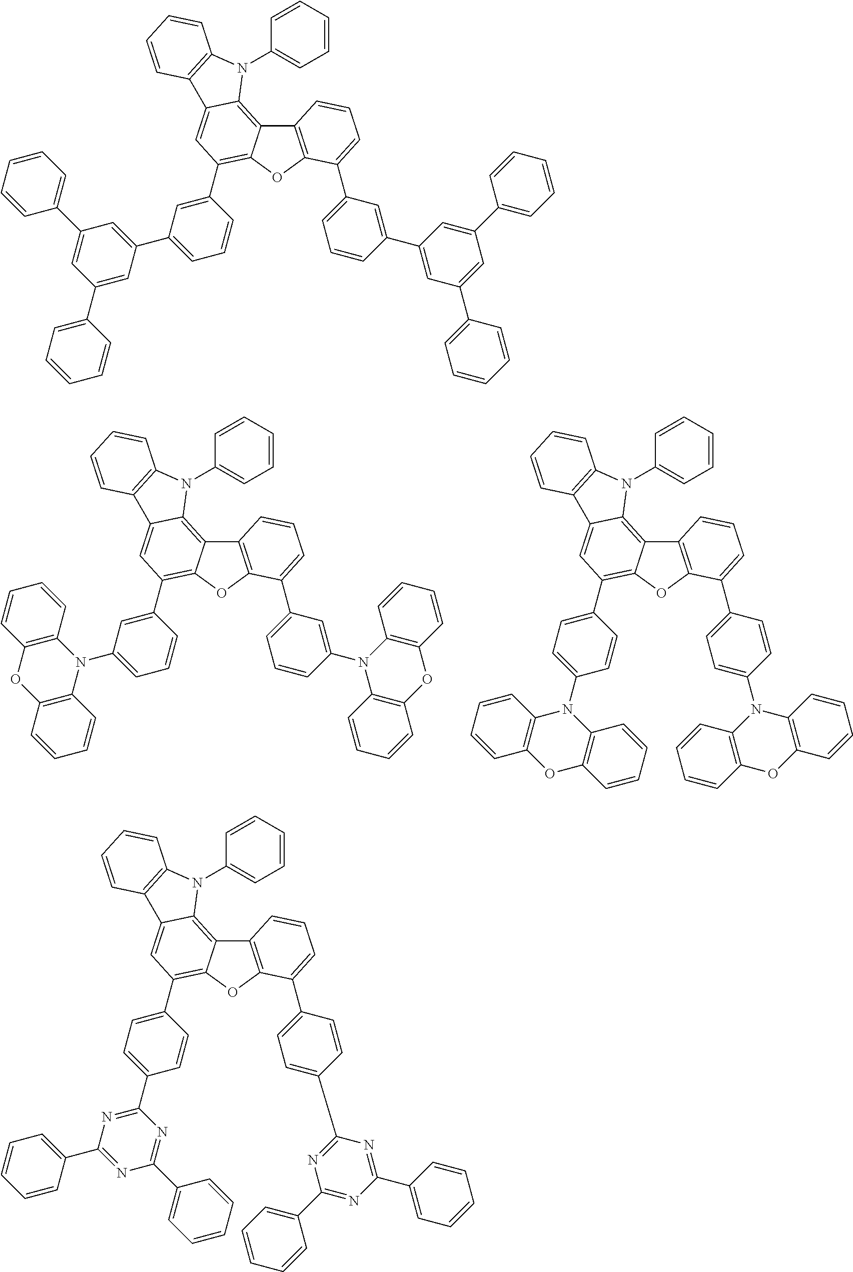

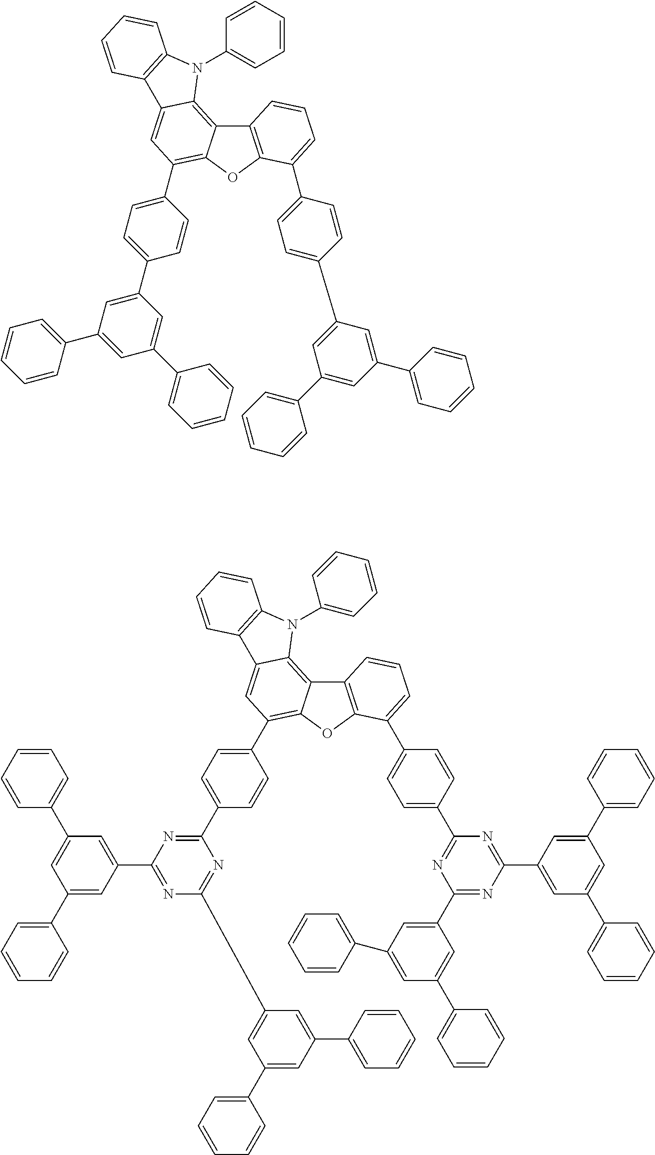

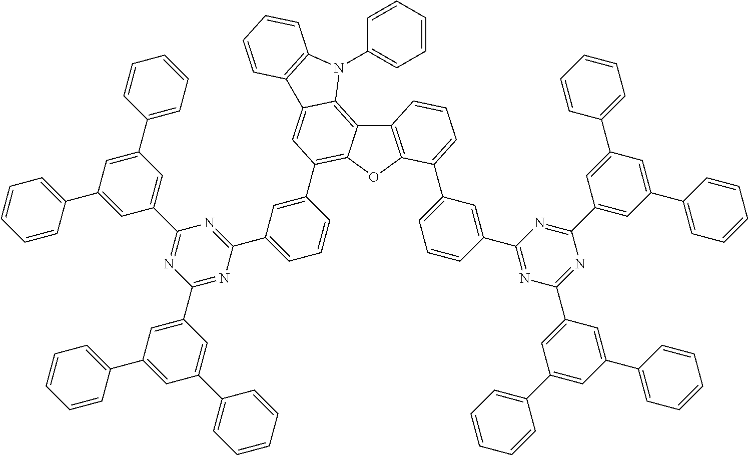

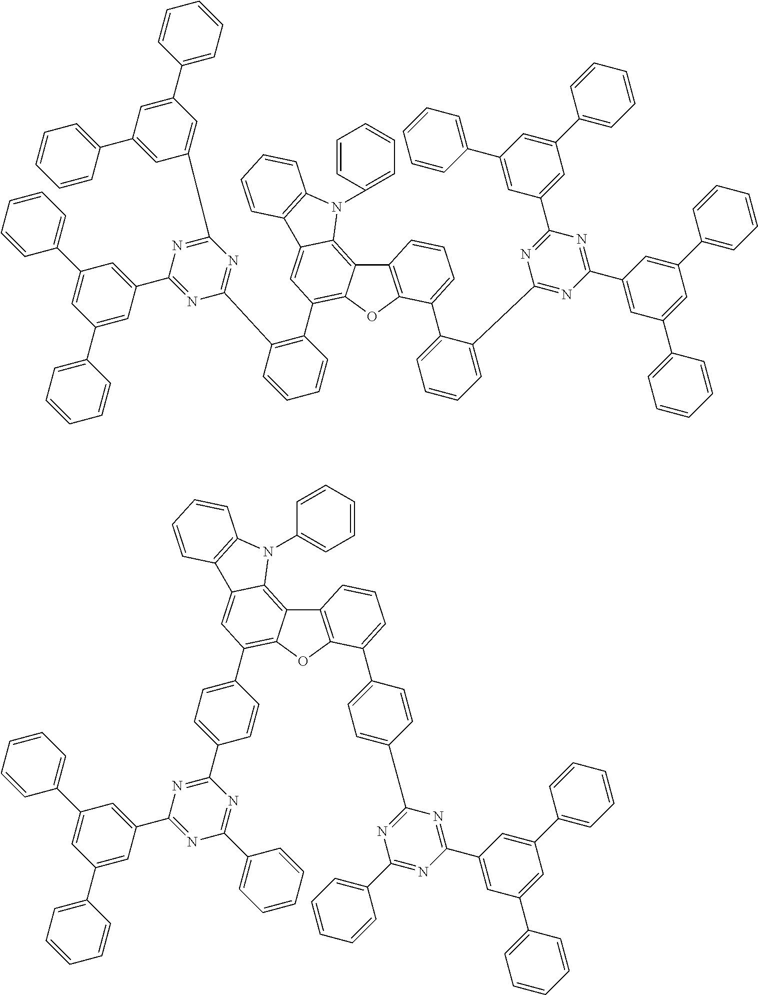

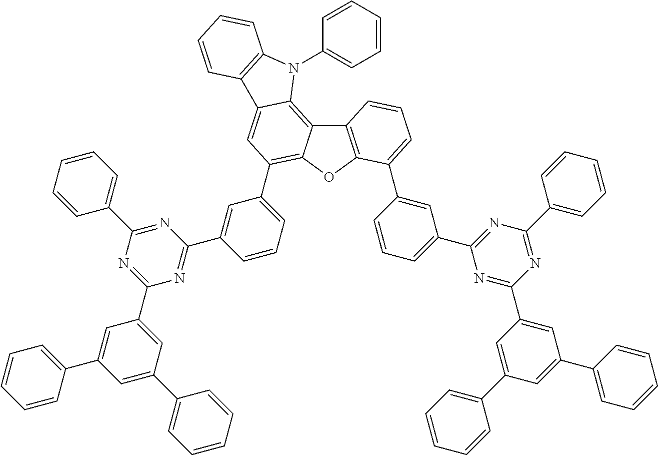

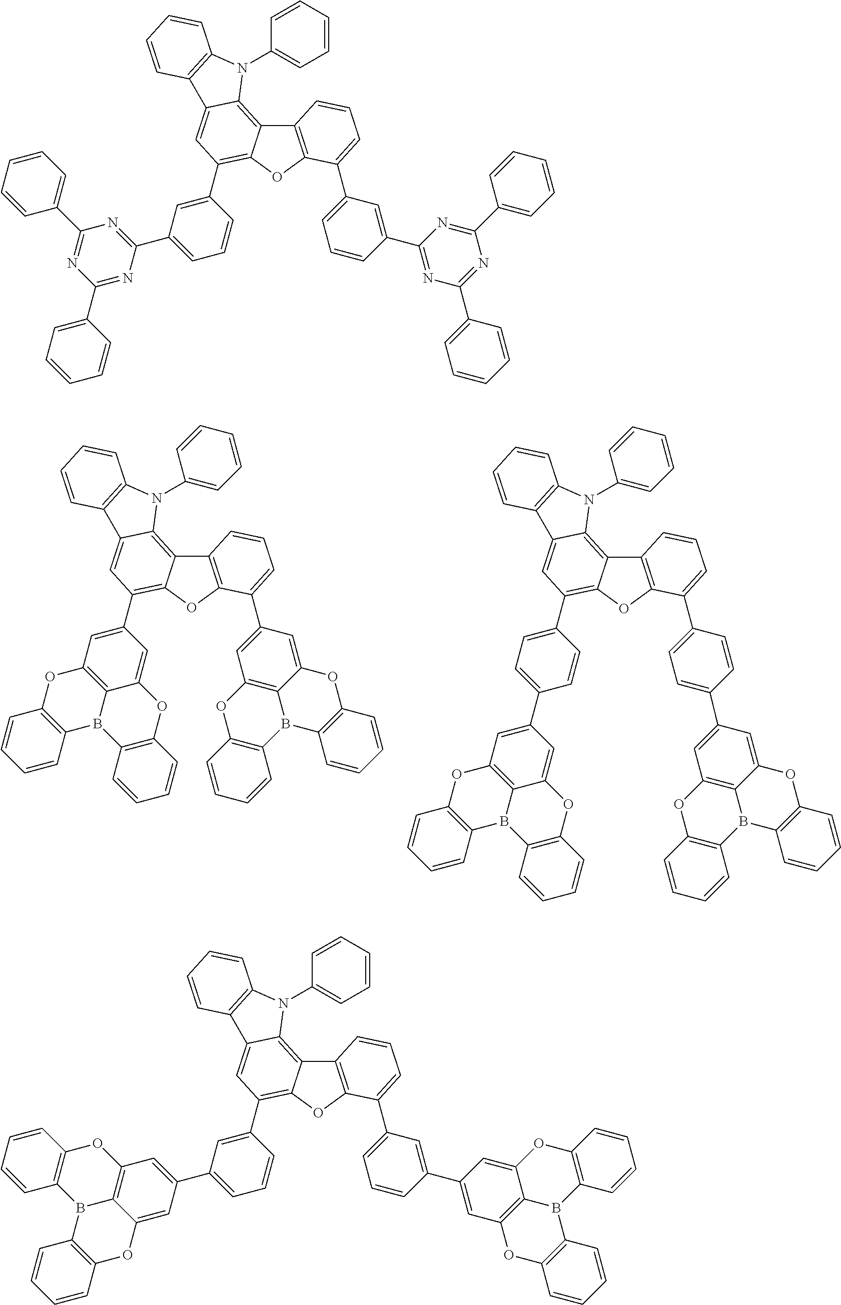

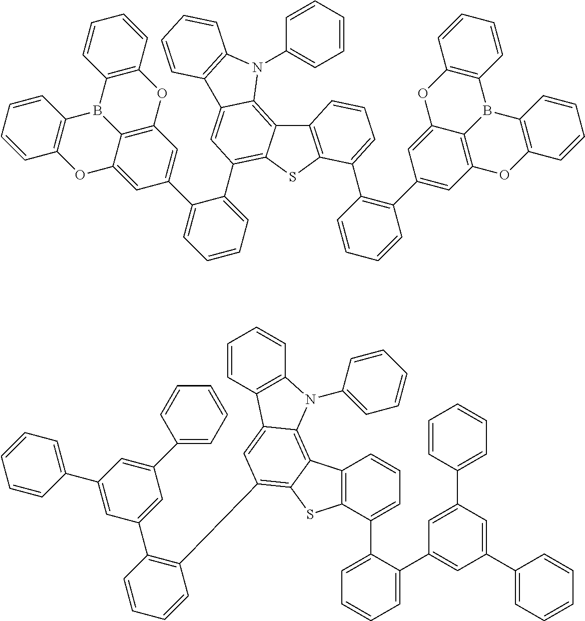

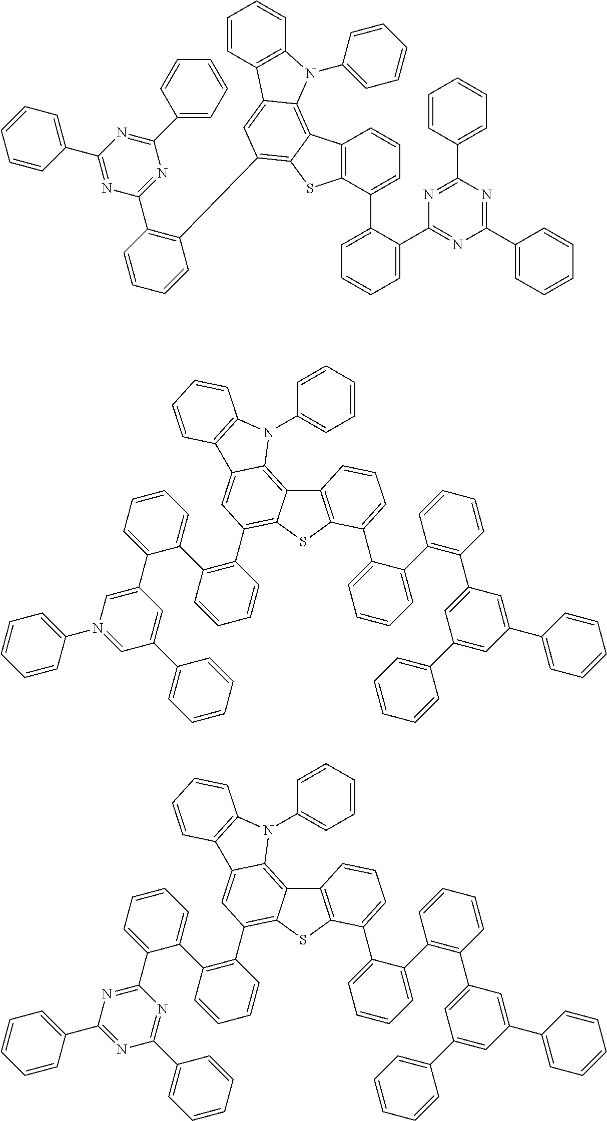

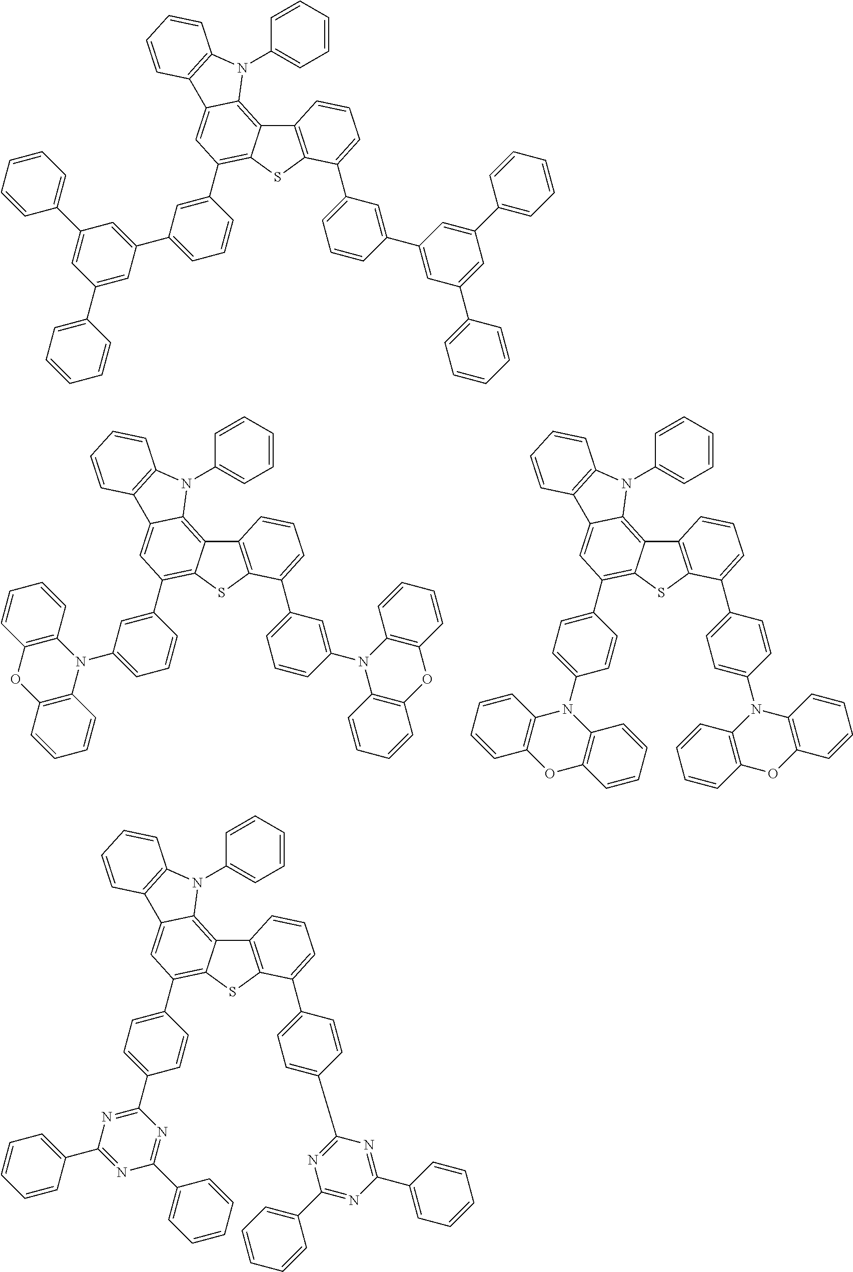

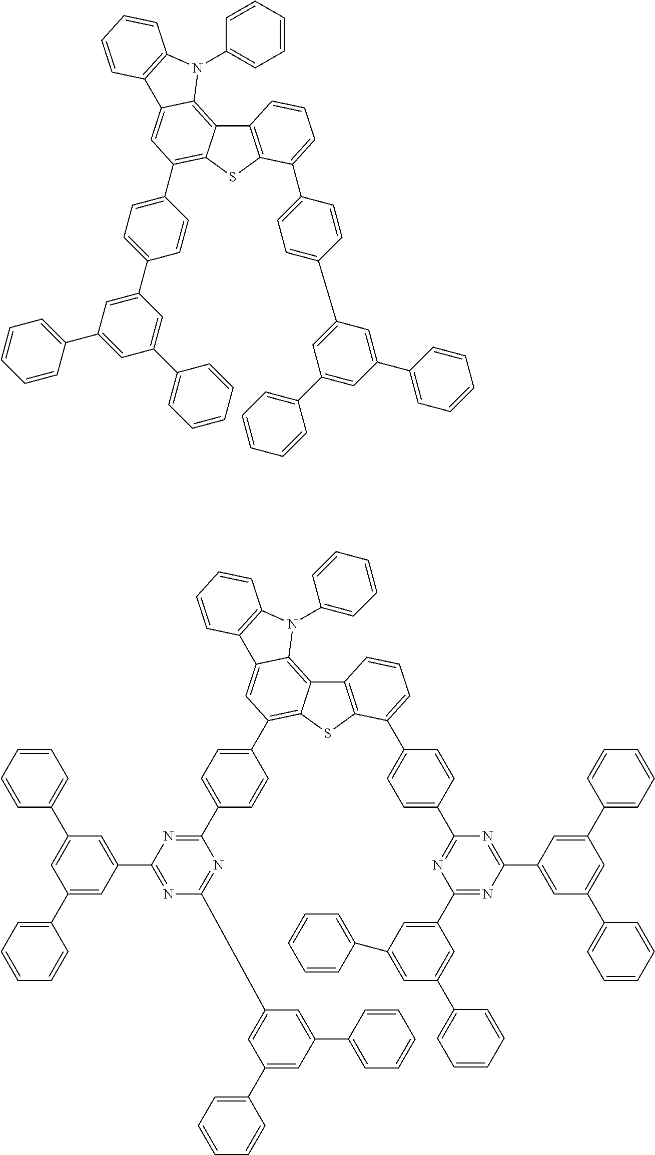

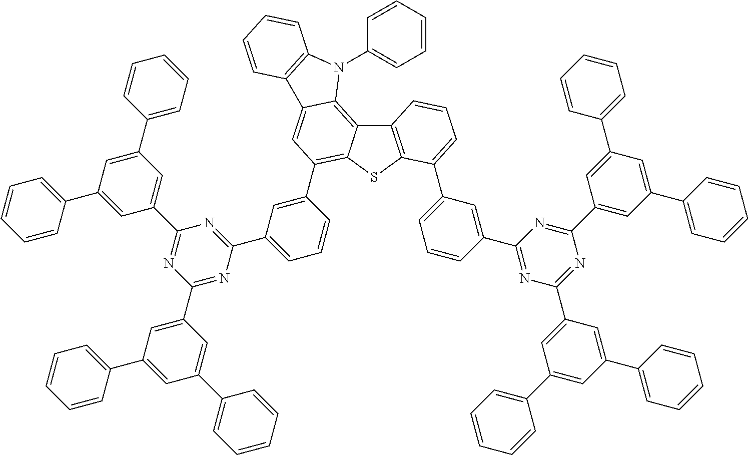

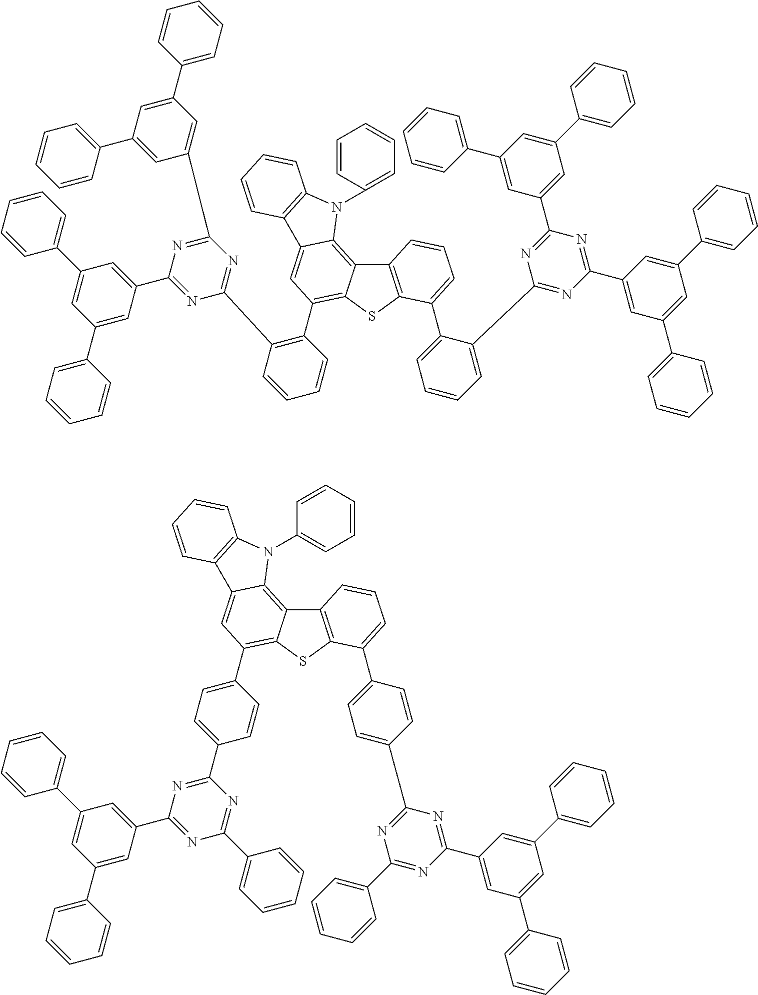

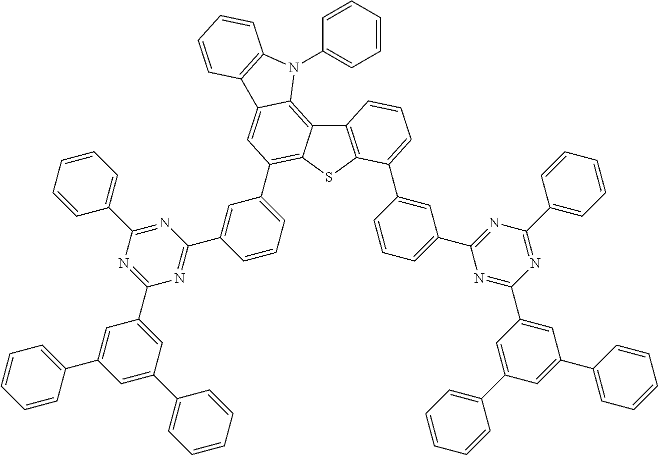

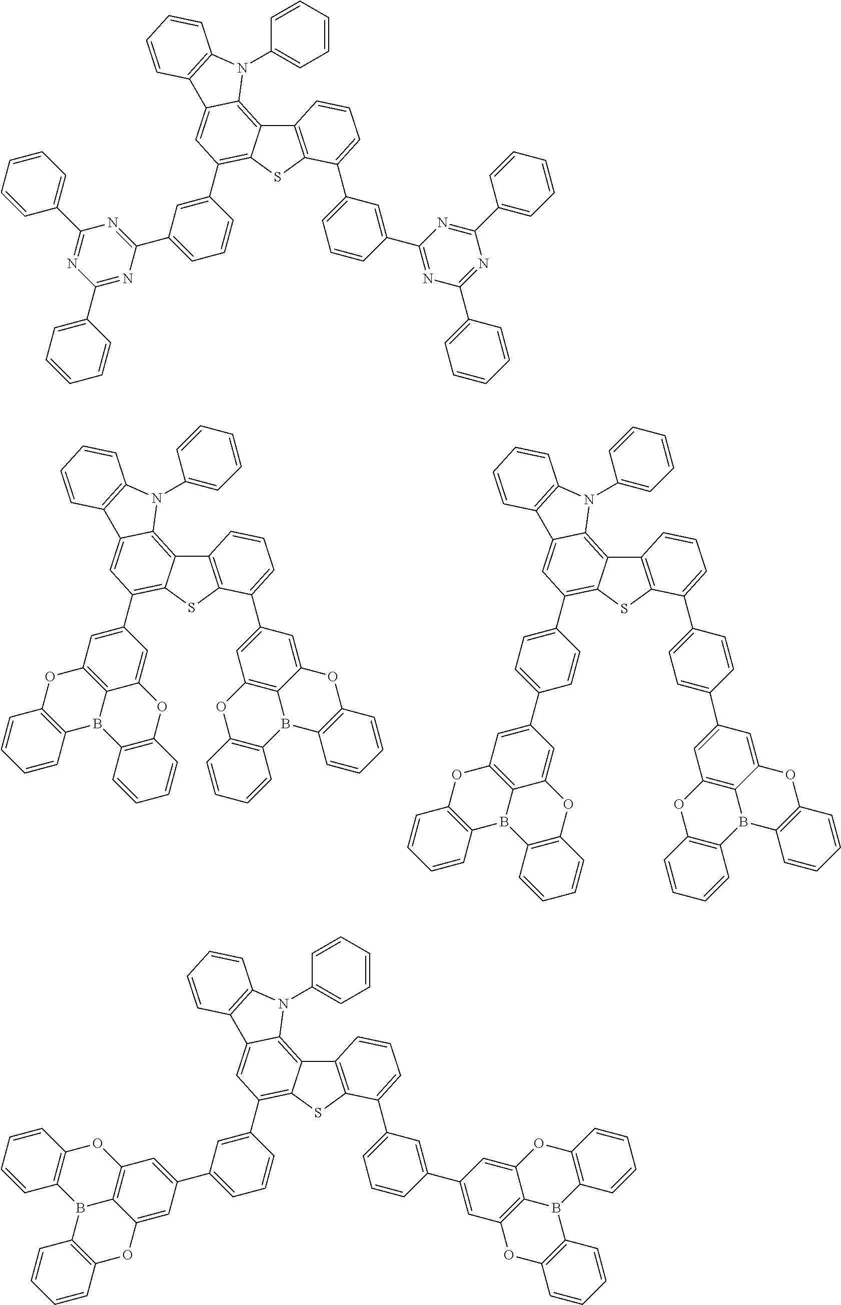

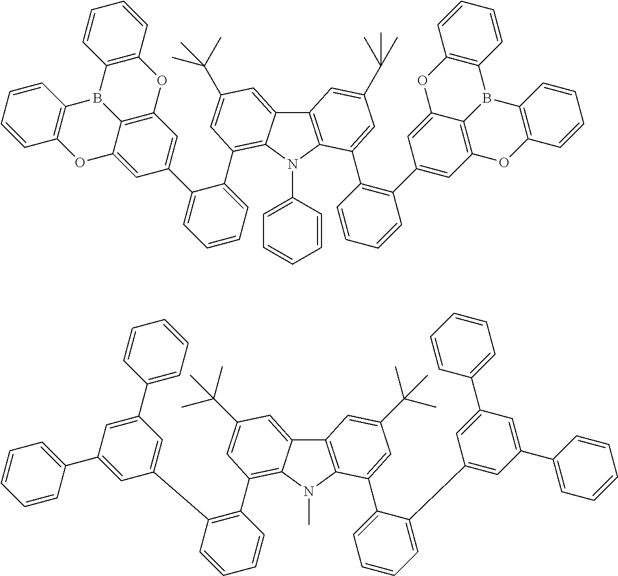

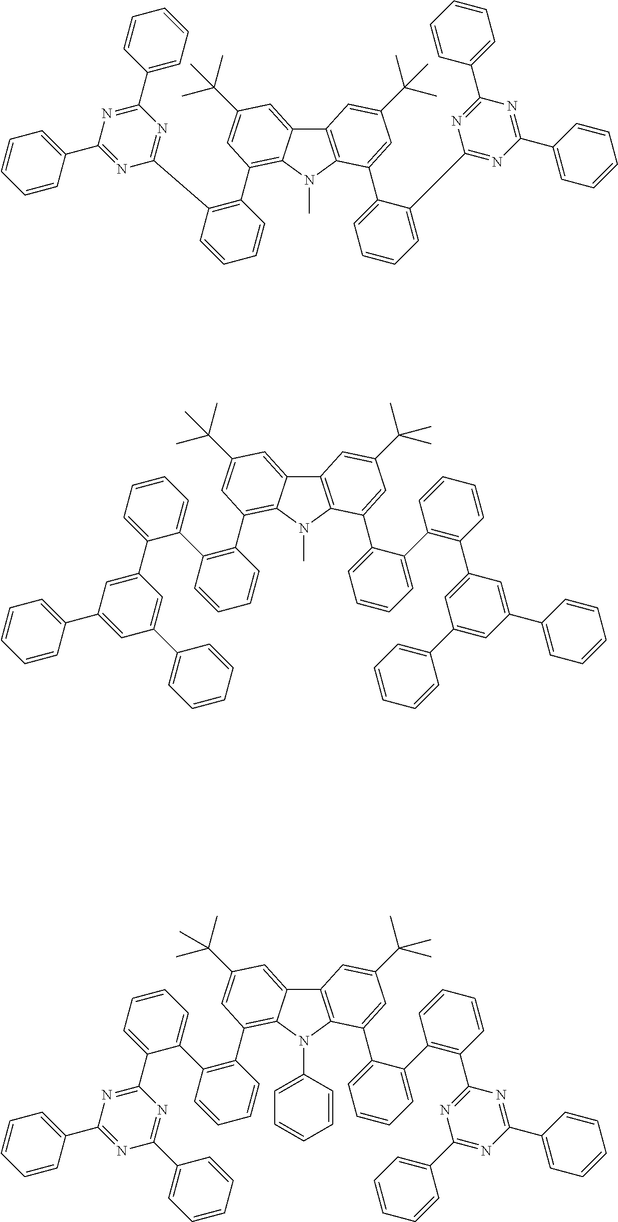

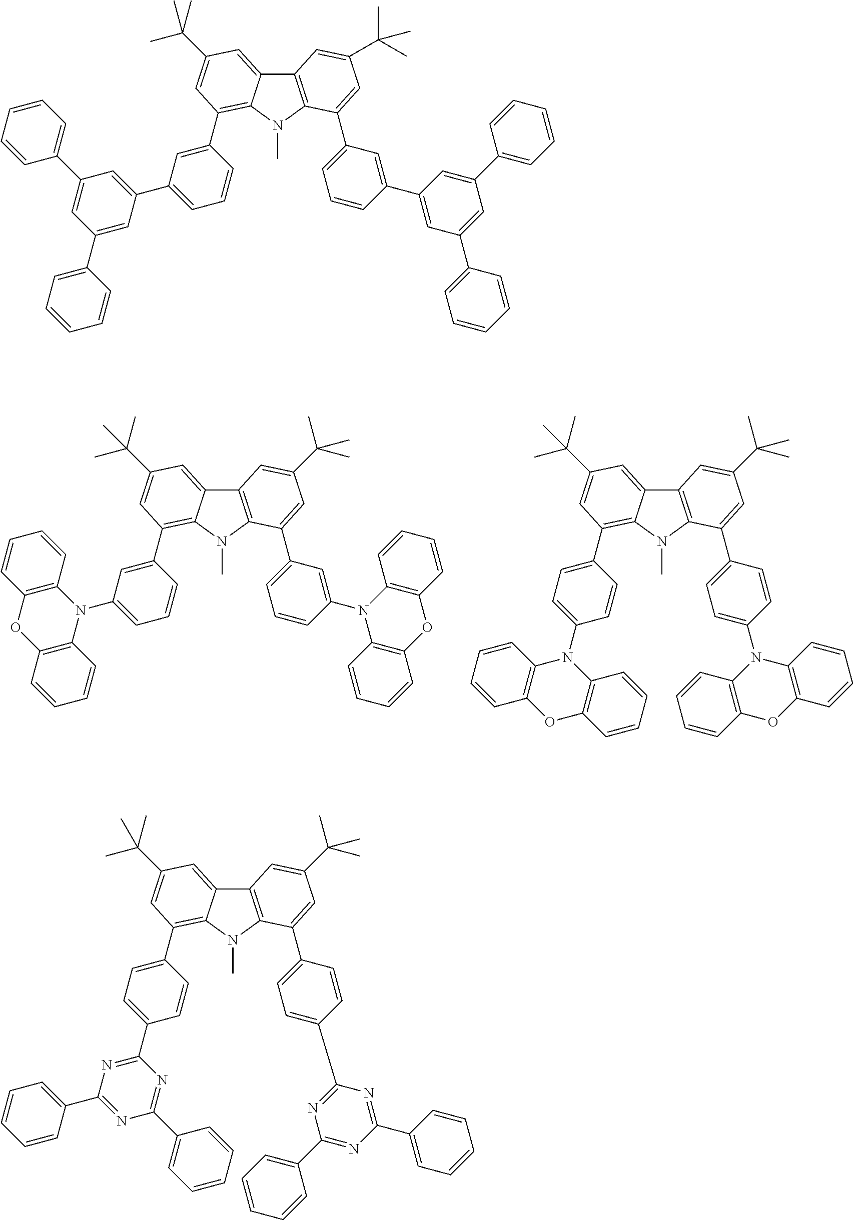

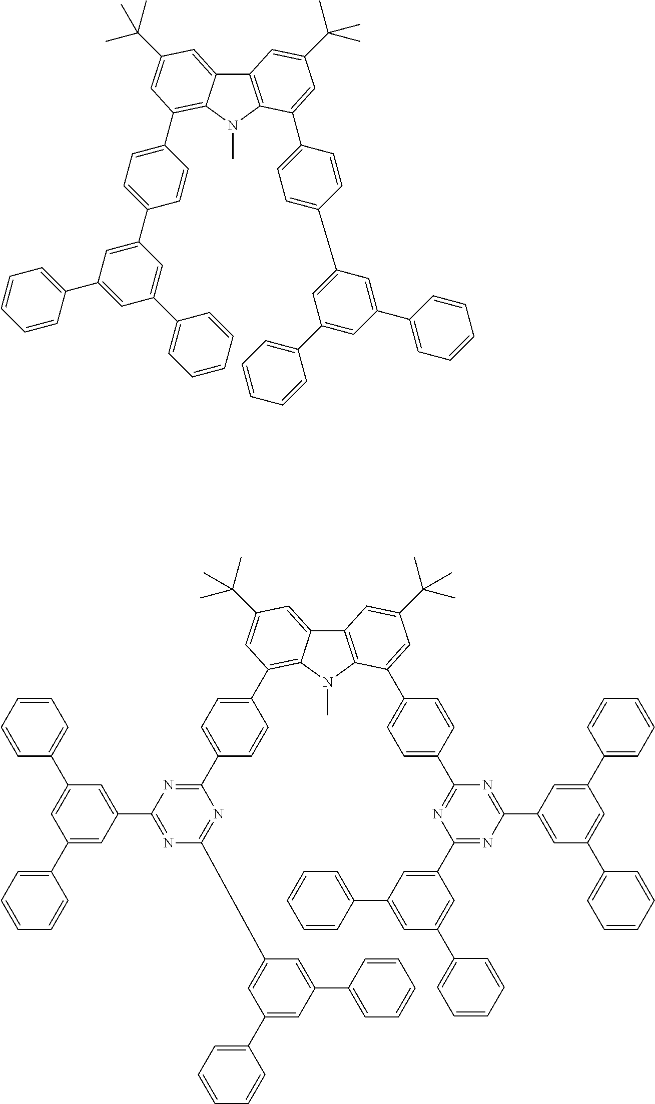

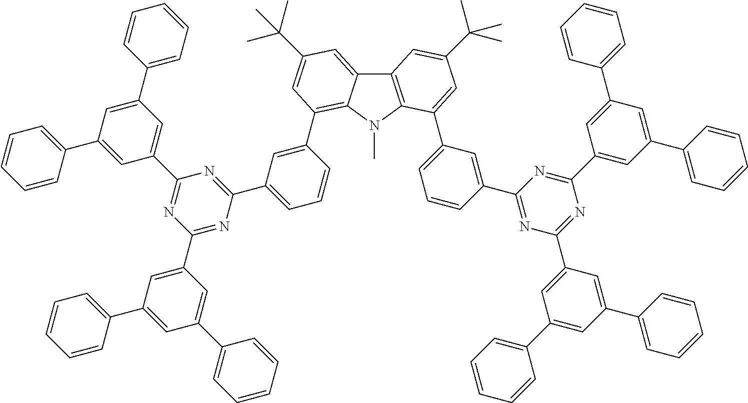

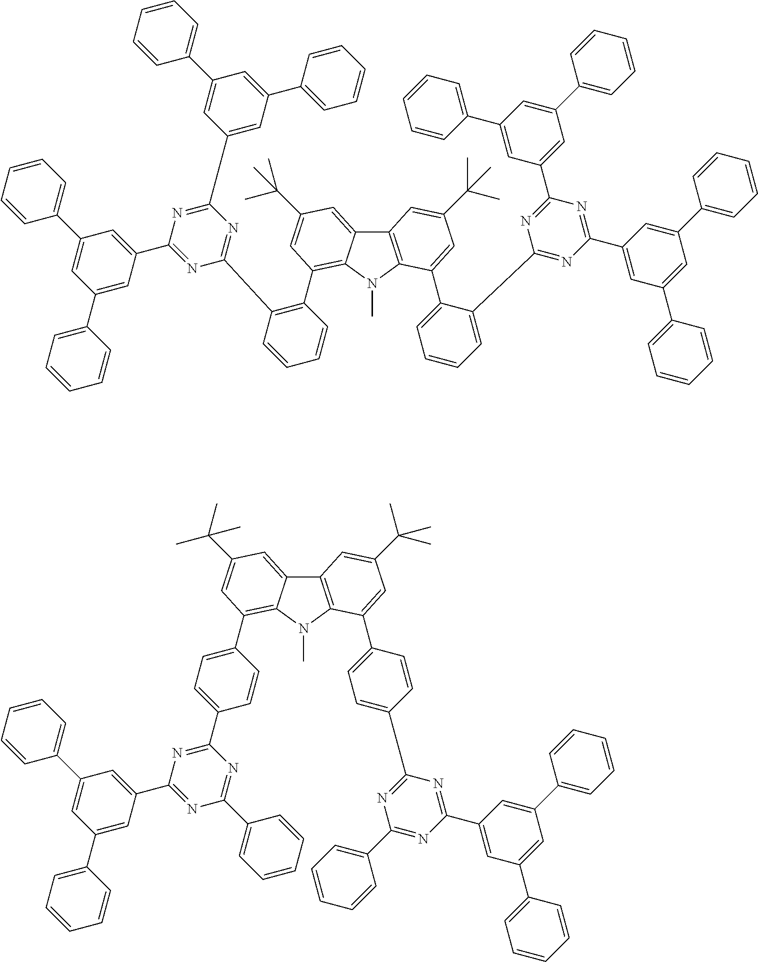

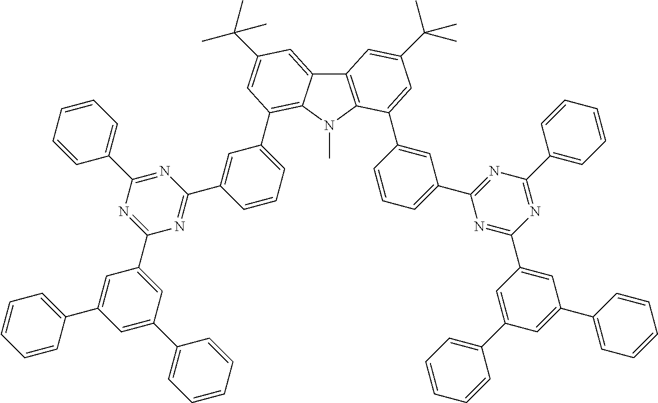

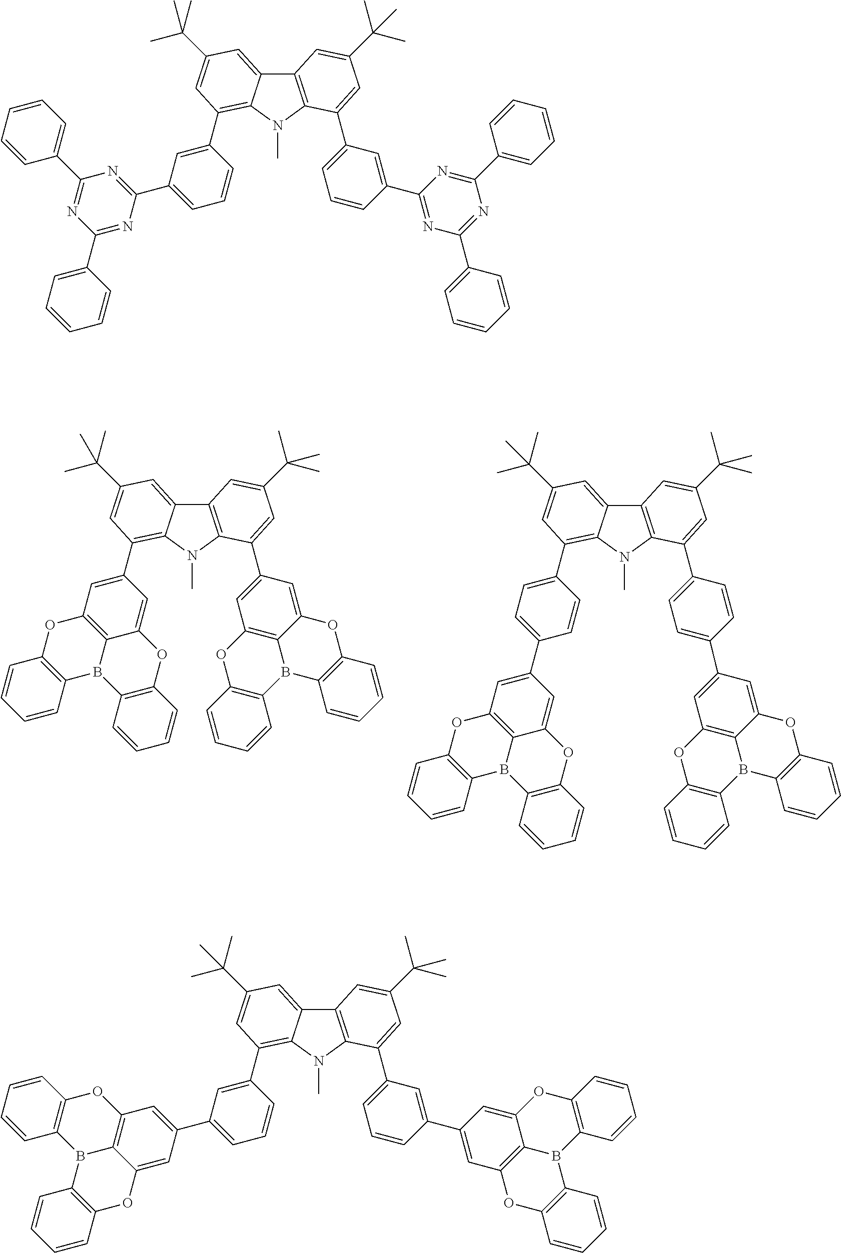

The present disclosure further relates to a polymer in which at least one repeating unit includes the structure represented by the general formula (1).

In one embodiment, the polymer is a non-conjugated polymer in which the structure unit represented by the general formula (1) is located on the side chain.

In another embodiment, the polymer is a conjugated polymer.

The present disclosure further relates to a mixture including the thermally activated delayed fluorescence material as mentioned above or the polymer as mentioned above, and at least one organic functional material.

The organic functional material includes a hole (also called an electron hole) injection material or hole transport material (HIM/HTM), a hole blocking material (HBM), an electron injection material or electron transport material (EIM/ETM), an electron blocking material (EBM), an organic host material (Host), a singlet emitter (fluorescent emitter), a thermally activated delayed fluorescence (TADF) material, and a triplet emitter (phosphorescent emitter), especially a luminescent organometallic complex, and an organic dye. These organic functional materials are described in detail, for example, in WO2010135519A1, US20090134784A1, and WO 2011110277A1. All contents of the three patent documents are specially incorporated herein by reference. The organic functional material may be a small molecule material or a polymer material.

In one embodiment, the mixture includes the thermally activated delayed fluorescence material as mentioned above or the polymer as mentioned above, and a phosphorescent emitter. The thermally activated delayed fluorescence material as mentioned above may be used as a host material herein. The weight percentage of the phosphorescent emitter is smaller than or equal to 30 wt %. In one embodiment, the weight percentage of the phosphorescent emitter is smaller than or equal to 25 wt %. In another embodiment, the weight percentage of the phosphorescent emitter is smaller than or equal to 20 wt %.

In one embodiment, the mixture includes the thermally activated delayed fluorescence material as mentioned above or the polymer as mentioned above, and a host material. The thermally activated delayed fluorescence material as mentioned above may be used as a luminescent material herein, and the weight percentage thereof is smaller than or equal to 30 wt % in one embodiment, is smaller than or equal to 25 wt % in another embodiment, is smaller than or equal to 20 wt % in another embodiment, and is smaller than or equal to 15 wt % in another embodiment.

In one embodiment, the mixture includes the thermally activated delayed fluorescence material as mentioned above or the polymer as mentioned above, a phosphorescent emitter, and a host material. The thermally activated delayed fluorescence material as mentioned above may be used as an auxiliary luminescent material herein. The weight ratio of the thermally activated delayed fluorescence material to the phosphorescent emitter is 1:2 to 2:1.

In one embodiment, T₁ of the thermally activated delayed fluorescence material as mentioned above is higher than T₁ of the phosphorescent emitter.

In one embodiment, the mixture includes the thermally activated delayed fluorescence material as mentioned above or the polymer as mentioned above, and another TADF material.

The following is a more detailed description of the host material, the phosphorescent material, and the TADF material (but not limited thereto).

1. Triplet Host Material (Triplet Host):

Examples of the triplet host are not particularly limited and any metal complex or organic compound can be used as the host material as long as its triplet energy is larger than that of the light emitter, especially than that of the triplet emitter or the phosphorescent emitter. Examples of metal complex that can be used as the triplet host can include, but are not limited to, the general structure as follows:

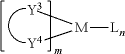

wherein M is a metal; (Y³-Y⁴) is a bidentate ligand, Y³ and Y⁴ are independently selected from C, N, O, P, and S; L is an auxiliary ligand; m is an integer from 1 to a maximum coordination number of the metal; and m+n is the maximum coordination number of the metal.

In one embodiment, the metal complex which can be used as the triplet host has one of the following formulas:

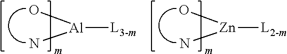

(O—N) is a bidentate ligand, and the metal is coordinated to the 0 and N atoms.

In one embodiment, M may be selected from Ir and Pt.

Examples of organic compounds that can be used as triplet host are selected from: compounds including cyclic aromatic hydrocarbon group, such as benzene, biphenyl, triphenyl, benzo, and fluorene; compounds including a heteroaromatic group, such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, oxazole, dibenzoxazole, benzisoxazole, benzothiazole, quinoline, isoquinoline, cinnoline, quinazoline, quinoxaline, naphthalene, phthalein, pteridine, xanthene, acridine, phenazine, phenothiazine, phenoxazine, benzofuropyridine, furopyridine, benzothienopyridine, thienopyridine, benzoselenolopyridine, and selenophene-benzodipyridine; and groups including 2 to 10 rings which may be the same or different cyclic aromatic hydrocarbon groups or heterocyclic aromatic groups, and may be linked to each other directly or by at least one of the following groups, such as an oxygen atom, a nitrogen atom, a sulfur atom, a silicon atom, a phosphorus atom, a boron atom, a chain structure unit, and an aliphatic ring. Each Ar can be further substituted and the substituents can be selected from hydrogen, alkyl, alkoxy, amino, alkenyl, alkynyl, aralkyl, heteroalkyl, aryl and heteroaryl.

In one embodiment, the triplet host material can be selected from compounds including at least one of the following groups:

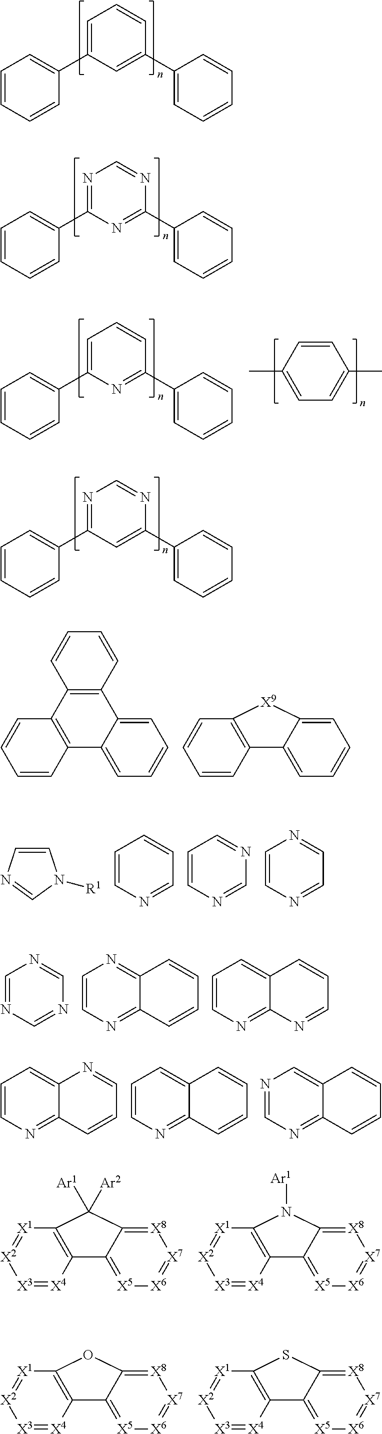

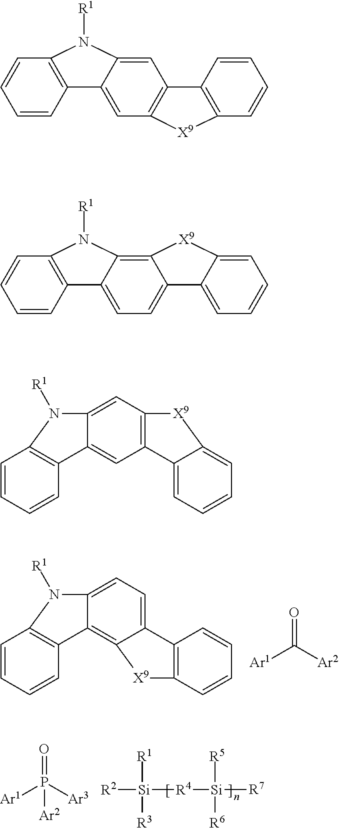

wherein R¹-R⁷ can be each independently selected from the group consisting of hydrogen, alkyl, alkoxy, amino, alkenyl, alkynyl, aralkyl, heteroalkyl, aryl, and heteroaryl; when R¹-R⁷ are aryl or heteroaryl, they have the same meaning as Ar¹ and Ar²; n is an integer from 0 to 20; X¹-X⁸ are each selected from CH or N; and X⁹ is selected from CR¹R² or NR¹.

Examples of suitable triplet host materials are listed in the following table:

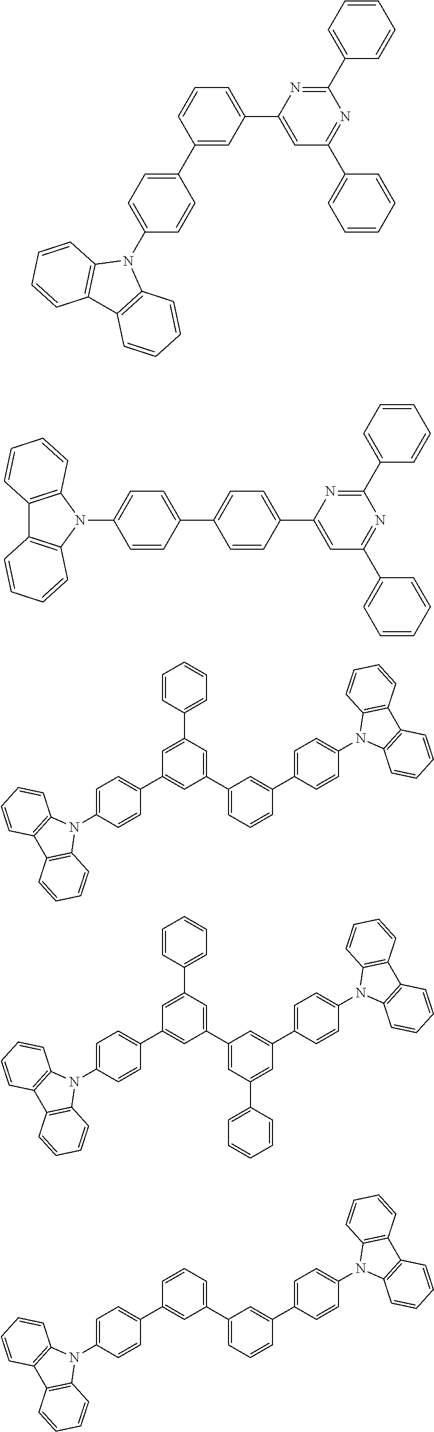

2. Phosphorescent Materials

The phosphorescent material is also called a triplet emitter.

In one embodiment, the triplet emitter is a metal complex having a general formula M(L)n, wherein M is a metal atom; L may be the same or different organic ligands in each occurrence, and may be bonded or coordinated to the metal atom M at one or more positions; n is an integer greater than 1, such as 1, 2, 3, 4, 5 or 6.

Optionally, these metal complexes are linked to a polymer at one or more positions, for example through the organic ligand.

In one embodiment, the metal atom M is selected from transition metal elements or lanthanides or actinides. In another embodiment, the metal atom M is selected from Ir, Pt, Pd, Au, Rh, Ru, Os, Sm, Eu, Gd, Tb, Dy, Re, Cu or Ag. In another embodiment, the metal atom M is selected from Os, Ir, Ru, Rh, Re, Pd, or Pt.

In one embodiment, the triplet emitter includes a chelate ligand, i.e., a ligand coordinated to the metal through at least two bonding sites.

In one embodiment, the triplet emitter includes two or three same or different bidentate ligands or polydentate ligands. The chelate ligands facilitates improving the stability of the metal complex.

Examples of the organic ligand may be selected from phenylpyridine derivatives, 7,8-benzoquinoline derivatives, 2(2-thienyl) pyridine derivatives, 2-(1-naphthyl) pyridine derivatives, or 2-phenylquinoline derivatives. All of these organic ligands may be substituted, for example, with fluoromethyl or trifluoromethyl. The auxiliary ligand can be selected from acetylacetone or picric acid.

In one embodiment, the metal complex which may be used as the triplet emitter has the following formula:

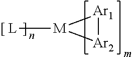

wherein M is a metal selected from transition metal elements, lanthanides, or actinides;

Ar¹ may be the same or different cyclic groups in each occurrence, which comprises at least one donor atom, that is, an atom with a lone pair of electrons, such as nitrogen atom or phosphorus atom, and the cyclic group is coordinately bonded to the metal through the donor atom;

Ar² may be the same or different cyclic groups in each occurrence, Ar² comprises at least one C atom and is linked to the metal through the C atom;

Ar¹ and Ar² are covalently bonded together, wherein each of them may carry one or more substituents, and they may also be linked together by the substituents;

L may be the same or different auxiliary ligands in each occurrence, In one embodiment, L is a bidentate chelate ligand. In one embodiment, L is a single canino bidentate chelate ligand;

m is 1, 2 or 3, such as 2 or 3. In one embodiment, m is 3;

n is 0, 1, or 2, such as 0 or 1. In one embodiment, n is 0.

The triplet emitter is also called a phosphorescent emitter.

In one embodiment, the triplet emitter is a metal complex having the general formula M(L)n, wherein M is a metal atom; L may be the same or different ligands in each occurrence, and may be bonded or coordinated to the metal atom M at one or more positions; n is an integer greater than 1, for example 1, 2, 3, 4, 5 or 6. In one embodiment, these metal complexes are linked to a polymer at one or more positions, for example through the organic ligand.

In one embodiment, the metal atom M is selected from transition metal elements or lanthanides or actinides. In one embodiment, the metal atom M is selected from Ir, Pt, Pd, Au, Rh, Ru, Os, Sm, Eu, Gd, Tb, Dy, Re, Cu or Ag. In another embodiment, the metal atom M is selected from Os, Ir, Ru, Rh, Re, Pd, or Pt.

In one embodiment, the triplet emitter comprises a chelate ligand, i.e., a ligand coordinated to the metal through at least two bonding sites.

In one embodiment, the triplet emitter includes two or three same or different bidentate ligands or polydentate ligands. The chelate ligands facilitates improving the stability of the metal complex.

Examples of organic ligands may be selected from phenylpyridine derivatives, 7,8-benzoquinoline derivatives, 2-(2-thienyl) pyridine derivatives, 2-(1-naphthyl) pyridine derivatives, or 2-phenylquinoline derivatives. All of these organic ligands may be substituted, for example, with fluoromethyl or trifluoromethyl. The auxiliary ligand can be selected from acetylacetone or picric acid.

In one embodiment, the metal complex which may be used as the triplet emitter has the following formula:

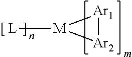

wherein M is a metal selected from transition metal elements, lanthanides, or actinides;

Ar¹ may be the same or different cyclic groups in each occurrence, which comprises at least one donor atom, that is, an atom with a lone pair of electrons, such as nitrogen atom or phosphorus atom, and the cyclic group is coordinately bonded to the metal through the donor atom;

Ar² may be the same or different cyclic groups in each occurrence, Ar² comprises at least one C atom and is linked to the metal through the C atom;

Ar¹ and Ar² are covalently bonded together, wherein each of them may carry one or more substituents, and they may also be linked together by the substituents;

L may be the same or different auxiliary ligand in each occurrence. In one embodiment, L is a bidentate chelate ligand. In one embodiment, L is a single canino bidentate chelate ligand;

m is 1, 2 or 3, such as 2 or 3. In one embodiment, m is 3;

n is 0, 1, or 2, such as 0 or 1. In one embodiment, n is 0.

Examples of the triplet emitter materials and their applications may be found in the following patent documents and references: WO 200070655, WO 200141512, WO 200202714, WO 200215645, EP 1191613, EP 1191612, EP 1191614, WO 2005033244, WO 2005019373, US 2005/0258742, WO 2009146770, WO 2010015307, WO 2010031485, WO 2010054731, WO 2010054728, WO 2010086089, WO 2010099852, WO 2010102709, US 20070087219 A1, US 20090061681 A1, US 20010053462 A1, Baldo, Thompson et al. Nature 403, (2000), 750-753, US 20090061681 A1, US 20090061681 A1, Adachi et al. Appl. Phys. Lett. 78 (2001), 1622-1624, J. Kido et al. Appl. Phys. Lett. 65 (1994), 2124, Kido et al. Chem. Lett. 657, 1990, US 2007/0252517 A1, Johnson et al., JACS 105, 1983, 1795, Wrighton, JACS 96, 1974, 998, Ma et al., Synth. Metals 94, 1998, 245, U.S. Pat. Nos. 6,824,895, 7,029,766, 6,835,469, 6,830,828, US 20010053462 A1, WO 2007095118 A1, US 2012004407A1, WO 2012007088A1, WO2012007087A1, WO 2012007086A1, US 2008027220A1, WO 2011157339A1, CN 102282150A, WO 2009118087A1. All contents of the patent documents and references listed above are specially incorporated herein by reference.

3. TADF Materials

Since the conventional organic fluorescence material can only use singlet state excitons accounting for 25% of the excitons excited by electricity to emit light, the internal quantum efficiency of the device is limited to 25%. The phosphorescent material can effectively use both singlet state excitons and triplet state excitons excited by electricity to emit light because of enhanced intersystem crossing due to strong spin-orbit coupling in centers of heavy atoms, so the internal quantum efficiency of the device may reach 100%. However, the application of the phosphorescent material in OLED is limited by its expensive price, poor stability, and severe roll-off device efficiency. The thermally activated delayed fluorescence material is a third generation of organic luminescent material developed after the organic fluorescence material and the organic phosphorescent material. Duo to small singlet state-triplet state energy level difference (ΔEst) of the TADF material, the triplet state excitons may be transformed to the singlet state excitons by converse intersystem crossing, thus the singlet state excitons and the triplet state excitons excited by electricity can be well utilized, and the internal quantum efficiency of the device may reach 100%. Meanwhile, the metal-free TADF material has a controllable structure, a stable property, and an inexpensive price, therefore may be widely used in OLED field.

The singlet state-triplet state energy level difference (ΔEst) of the TADF material should be small. In one embodiment, the singlet state-triplet state energy level difference (ΔEst) of the TADF material is smaller than 0.3 eV. In another embodiment, the singlet state-triplet state energy level difference (ΔEst) of the TADF material is smaller than 0.2 eV. In another embodiment, the singlet state-triplet state energy level difference (ΔEst) of the TADF material is smaller than 0.1 eV.

In one embodiment, the TADF material has a small ΔEst. In another embodiment, the TADF material has a high fluorescent quantum efficiency. Some TADF materials may be found in the following patent documents CN103483332(A), TW201309696(A), TW201309778(A), TW201343874(A), TW201350558(A), US20120217869(A1), WO2013133359(A1), WO2013154064(A1), Adachi, et. al. Adv. Mater., 21, 2009, 4802, Adachi, et. al. Appl. Phys. Lett., 98, 2011, 083302, Adachi, et. al. Appl. Phys. Lett., 101, 2012, 093306, Adachi, et. al. Chem. Commun., 48, 2012, 11392, Adachi, et. al. Nature Photonics, 6, 2012, 253, Adachi, et. al. Nature, 492, 2012, 234, Adachi, et. al. J. Am. Chem. Soc, 134, 2012, 14706, Adachi, et. al. Angew. Chem. Int. Ed, 51, 2012, 11311, Adachi, et. al. Chem. Commun., 48, 2012, 9580, Adachi, et. al. Chem. Commun., 48, 2013, 10385, Adachi, et. al. Adv. Mater., 25, 2013, 3319, Adachi, et. al. Adv. Mater., 25, 2013, 3707, Adachi, et. al. Chem. Mater., 25, 2013, 3038, Adachi, et. al. Chem. Mater., 25, 2013, 3766, Adachi, et. al. J. Mater. Chem. C., 1, 2013, 4599, Adachi, et. al. J. Phys. Chem. A., 117, 2013, 5607, all contents of which are specially incorporated herein by reference.

Examples of suitable TADF materials are listed in the following table:

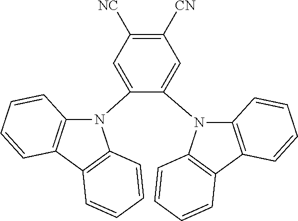 [SMILES: N#Cc1cc(-n2c3ccccc3c3ccccc32)c(-n2c3ccccc3c3ccccc32)cc1C#N]

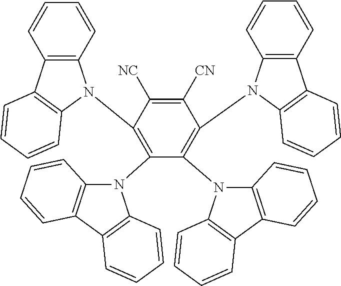 [SMILES: N#Cc1c(C#N)c(-n2c3ccccc3c3ccccc32)c(-n2c3ccccc3c3ccccc32)c(-n2c3ccccc3c3ccccc32)c1-n1c2ccccc2c2ccccc21]

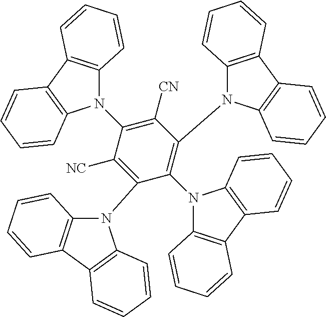 [SMILES: N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

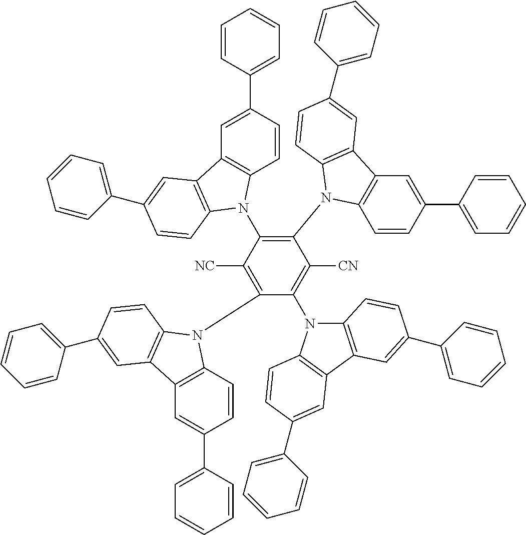 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21]

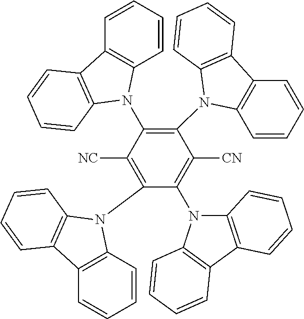 [SMILES: N#Cc1c(-n2c3ccccc3c3ccccc32)c(-n2c3ccccc3c3ccccc32)c(C#N)c(-n2c3ccccc3c3ccccc32)c1-n1c2ccccc2c2ccccc21]

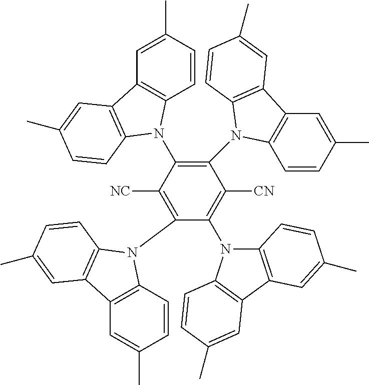

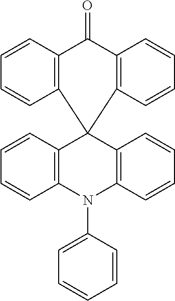 [SMILES: O=C1c2ccccc2C2(c3ccccc31)c1ccccc1N(c1ccccc1)c1ccccc12]

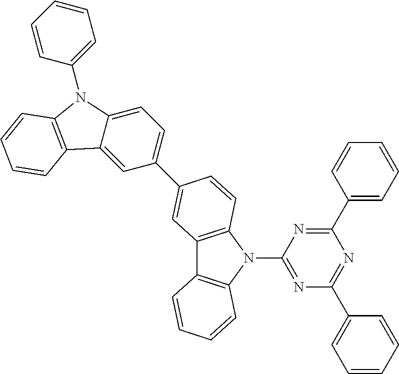 [SMILES: c1ccc(-c2nc(-c3ccccc3)nc(-n3c4ccccc4c4cc(-c5ccc6c(c5)c5ccccc5n6-c5ccccc5)ccc43)n2)cc1]

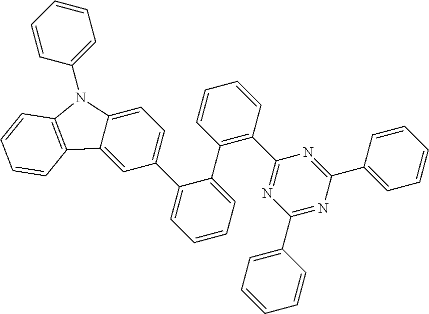 [SMILES: c1ccc(-c2nc(-c3ccccc3)nc(-c3ccccc3-c3ccccc3-c3ccc4c(c3)c3ccccc3n4-c3ccccc3)n2)cc1]

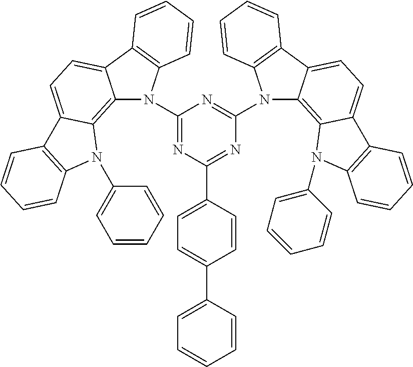 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1]

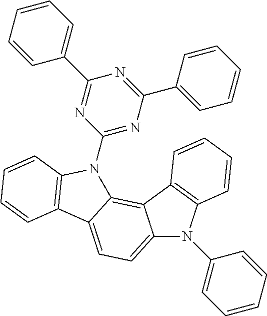 [SMILES: c1ccc(-c2nc(-c3ccccc3)nc(-n3c4ccccc4c4ccc5c(c6ccccc6n5-c5ccccc5)c43)n2)cc1]

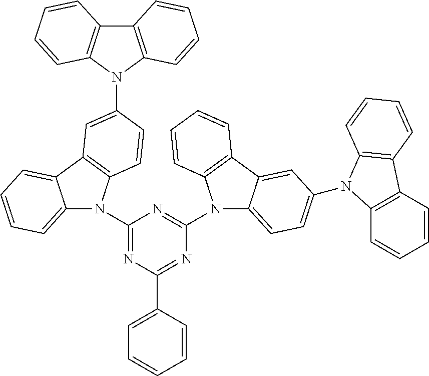 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1]

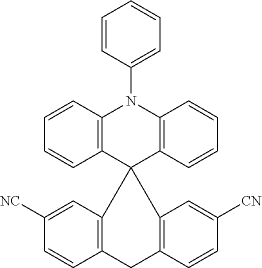 [SMILES: N#Cc1ccc2c(c1)C1(c3cc(C#N)ccc3C2)c2ccccc2N(c2ccccc2)c2ccccc21]

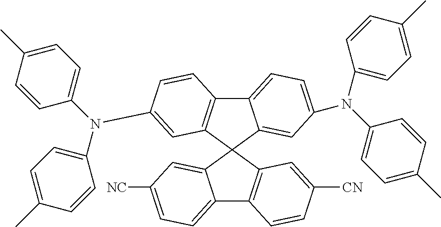

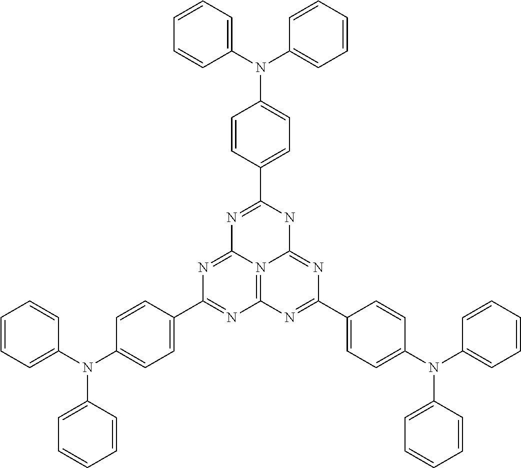

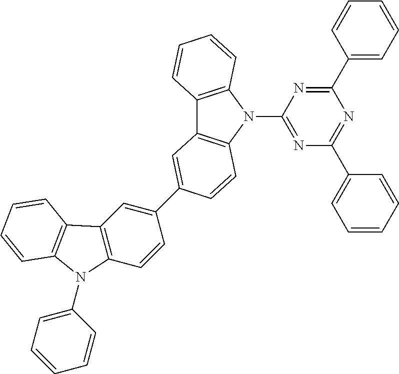 [SMILES: c1ccc(-c2nc(-c3ccccc3)nc(-n3c4ccccc4c4cc(-c5ccc6c(c5)c5ccccc5n6-c5ccccc5)ccc43)n2)cc1]

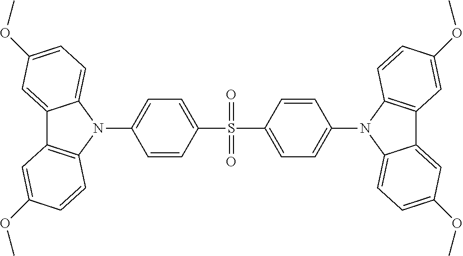 [SMILES: COc1ccc2c(c1)c1cc(OC)ccc1n2-c1ccc(S(=O)(=O)c2ccc(-n3c4ccc(OC)cc4c4cc(OC)ccc43)cc2)cc1]

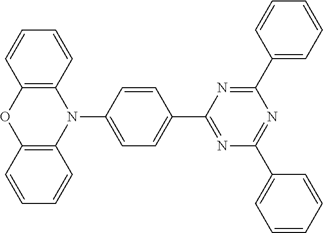 [SMILES: c1ccc(-c2nc(-c3ccccc3)nc(-c3ccc(N4c5ccccc5Oc5ccccc54)cc3)n2)cc1]

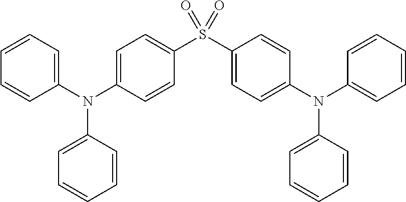 [SMILES: O=S(=O)(c1ccc(N(c2ccccc2)c2ccccc2)cc1)c1ccc(N(c2ccccc2)c2ccccc2)cc1]

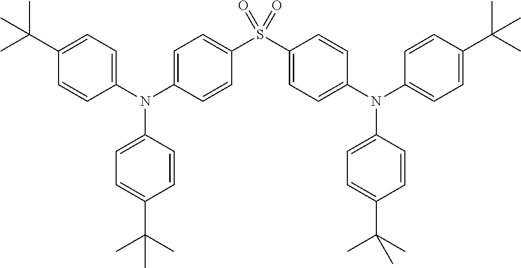 [SMILES: CC(C)(C)c1ccc(N(c2ccc(C(C)(C)C)cc2)c2ccc(S(=O)(=O)c3ccc(N(c4ccc(C(C)(C)C)cc4)c4ccc(C(C)(C)C)cc4)cc3)cc2)cc1]

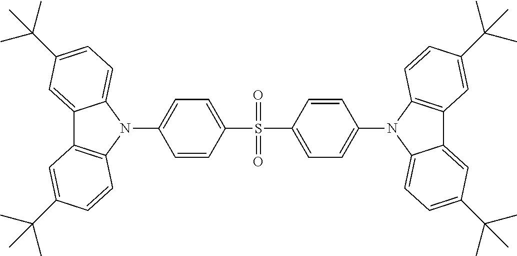 [SMILES: CC(C)(C)c1ccc2c(c1)c1cc(C(C)(C)C)ccc1n2-c1ccc(S(=O)(=O)c2ccc(-n3c4ccc(C(C)(C)C)cc4c4cc(C(C)(C)C)ccc43)cc2)cc1]

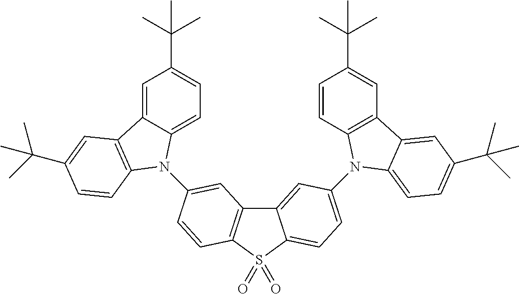 [SMILES: CC(C)(C)c1ccc2c(c1)c1cc(C(C)(C)C)ccc1n2-c1ccc2c(c1)-c1cc(-n3c4ccc(C(C)(C)C)cc4c4cc(C(C)(C)C)ccc43)ccc1S2(=O)=O]

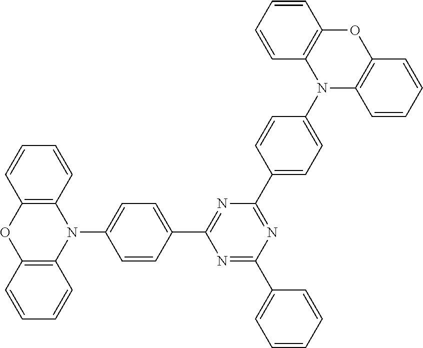 [SMILES: c1ccc(-c2nc(-c3ccc(N4c5ccccc5Oc5ccccc54)cc3)nc(-c3ccc(N4c5ccccc5Oc5ccccc54)cc3)n2)cc1]

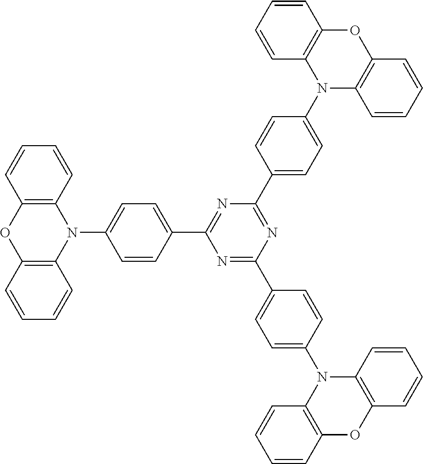 [SMILES: c1ccc2c(c1)Oc1ccccc1N2c1ccc(-c2nc(-c3ccc(N4c5ccccc5Oc5ccccc54)cc3)nc(-c3ccc(N4c5ccccc5Oc5ccccc54)cc3)n2)cc1]

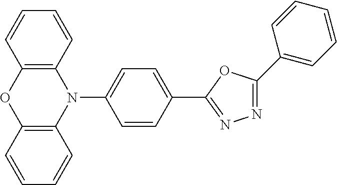 [SMILES: c1ccc(-c2nnc(-c3ccc(N4c5ccccc5Oc5ccccc54)cc3)o2)cc1]

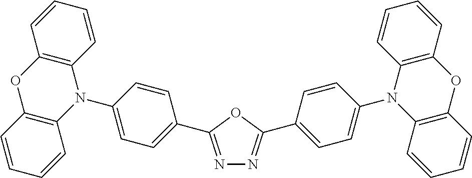 [SMILES: c1ccc2c(c1)Oc1ccccc1N2c1ccc(-c2nnc(-c3ccc(N4c5ccccc5Oc5ccccc54)cc3)o2)cc1]

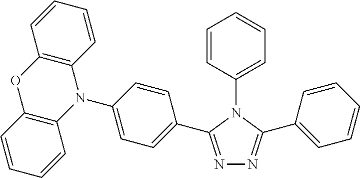 [SMILES: c1ccc(-c2nnc(-c3ccc(N4c5ccccc5Oc5ccccc54)cc3)n2-c2ccccc2)cc1]

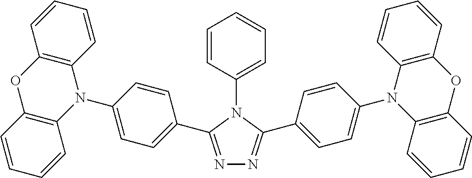 [SMILES: c1ccc(-n2c(-c3ccc(N4c5ccccc5Oc5ccccc54)cc3)nnc2-c2ccc(N3c4ccccc4Oc4ccccc43)cc2)cc1]

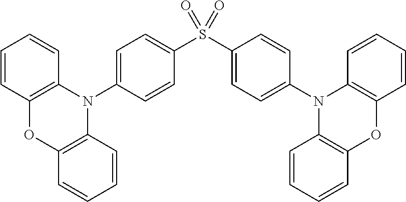 [SMILES: O=S(=O)(c1ccc(N2c3ccccc3Oc3ccccc32)cc1)c1ccc(N2c3ccccc3Oc3ccccc32)cc1]

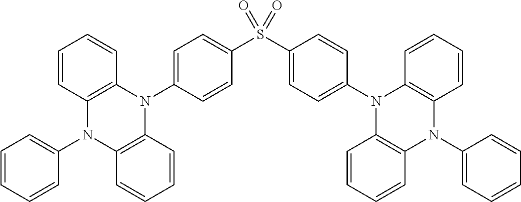 [SMILES: O=S(=O)(c1ccc(N2c3ccccc3N(c3ccccc3)c3ccccc32)cc1)c1ccc(N2c3ccccc3N(c3ccccc3)c3ccccc32)cc1]

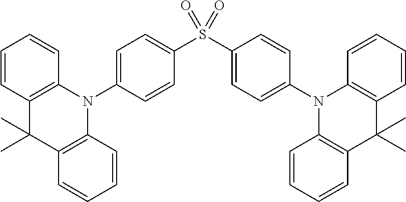 [SMILES: CC1(C)c2ccccc2N(c2ccc(S(=O)(=O)c3ccc(N4c5ccccc5C(C)(C)c5ccccc54)cc3)cc2)c2ccccc21]

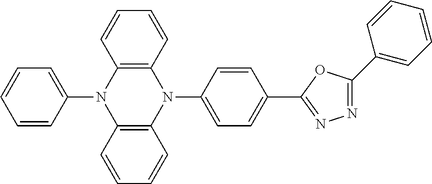 [SMILES: c1ccc(-c2nnc(-c3ccc(N4c5ccccc5N(c5ccccc5)c5ccccc54)cc3)o2)cc1]

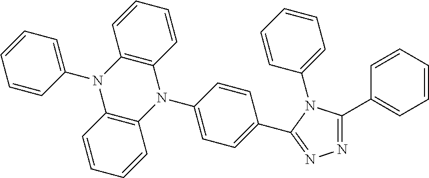 [SMILES: c1ccc(-c2nnc(-c3ccc(N4c5ccccc5N(c5ccccc5)c5ccccc54)cc3)n2-c2ccccc2)cc1]

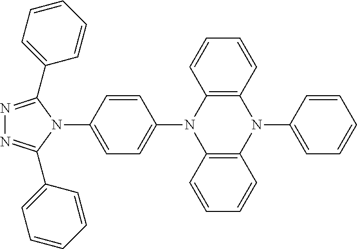 [SMILES: c1ccc(-c2nnc(-c3ccccc3)n2-c2ccc(N3c4ccccc4N(c4ccccc4)c4ccccc43)cc2)cc1]

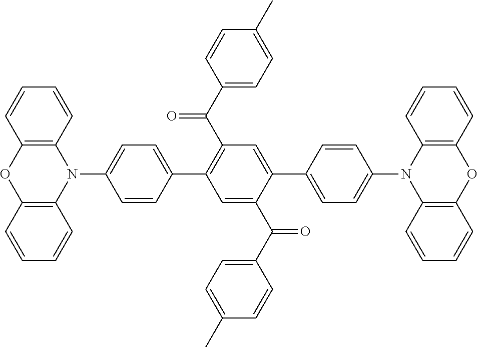 [SMILES: Cc1ccc(C(=O)c2cc(-c3ccc(N4c5ccccc5Oc5ccccc54)cc3)c(C(=O)c3ccc(C)cc3)cc2-c2ccc(N3c4ccccc4Oc4ccccc43)cc2)cc1]

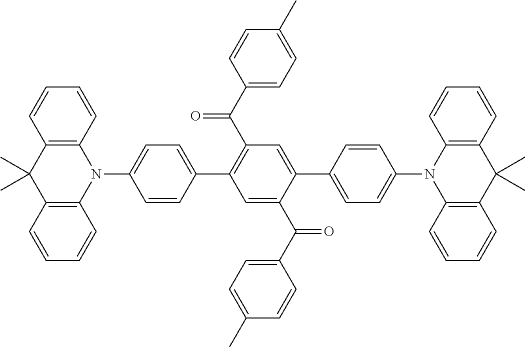

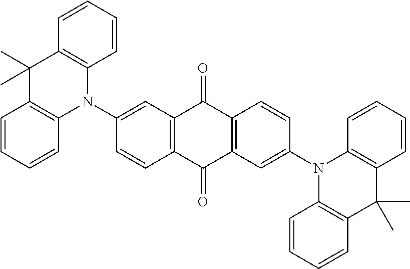 [SMILES: CC1(C)c2ccccc2N(c2ccc3c(c2)C(=O)c2ccc(N4c5ccccc5C(C)(C)c5ccccc54)cc2C3=O)c2ccccc21]

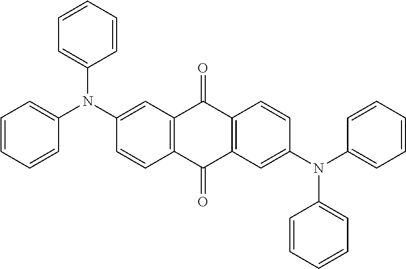 [SMILES: O=C1c2ccc(N(c3ccccc3)c3ccccc3)cc2C(=O)c2ccc(N(c3ccccc3)c3ccccc3)cc21]

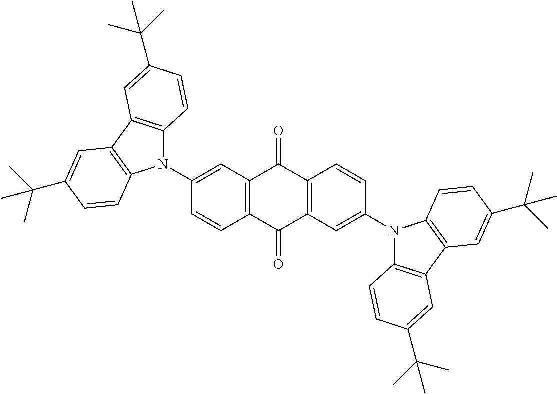 [SMILES: CC(C)(C)c1ccc2c(c1)c1cc(C(C)(C)C)ccc1n2-c1ccc2c(c1)C(=O)c1ccc(-n3c4ccc(C(C)(C)C)cc4c4cc(C(C)(C)C)ccc43)cc1C2=O]

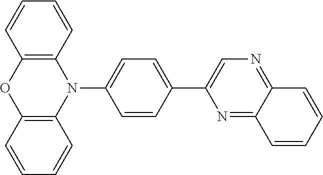 [SMILES: c1ccc2c(c1)Oc1ccccc1N2c1ccc(-c2cnc3ccccc3n2)cc1]

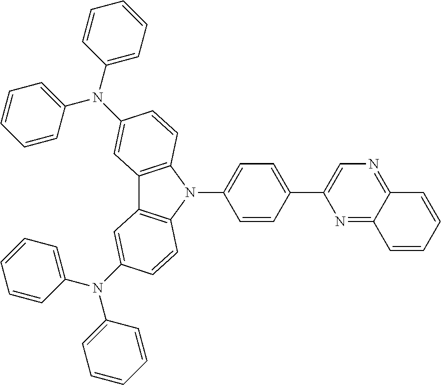 [SMILES: c1ccc(N(c2ccccc2)c2ccc3c(c2)c2cc(N(c4ccccc4)c4ccccc4)ccc2n3-c2ccc(-c3cnc4ccccc4n3)cc2)cc1]

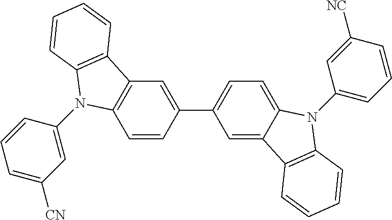 [SMILES: N#Cc1cccc(-n2c3ccccc3c3cc(-c4ccc5c(c4)c4ccccc4n5-c4cccc(C#N)c4)ccc32)c1]

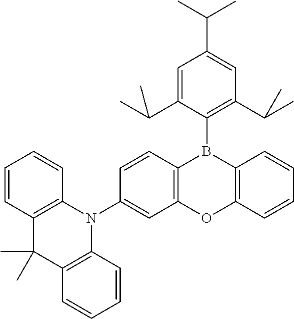 [SMILES: CC(C)c1cc(C(C)C)c(B2c3ccccc3Oc3cc(N4c5ccccc5C(C)(C)c5ccccc54)ccc32)c(C(C)C)c1]

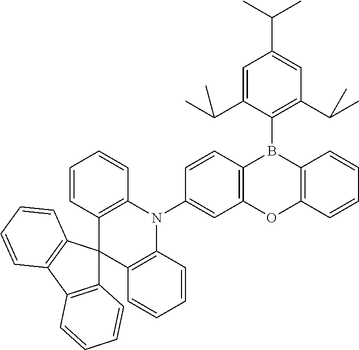 [SMILES: CC(C)c1cc(C(C)C)c(B2c3ccccc3Oc3cc(N4c5ccccc5C5(c6ccccc6-c6ccccc65)c5ccccc54)ccc32)c(C(C)C)c1]

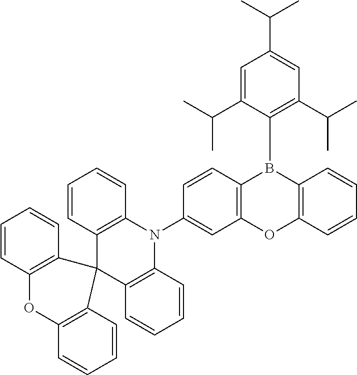 [SMILES: CC(C)c1cc(C(C)C)c(B2c3ccccc3Oc3cc(N4c5ccccc5C5(c6ccccc6Oc6ccccc65)c5ccccc54)ccc32)c(C(C)C)c1]

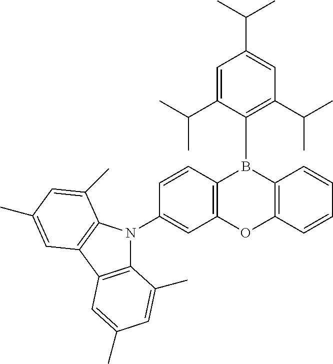 [SMILES: Cc1cc(C)c2c(c1)c1cc(C)cc(C)c1n2-c1ccc2c(c1)Oc1ccccc1B2c1c(C(C)C)cc(C(C)C)cc1C(C)C]

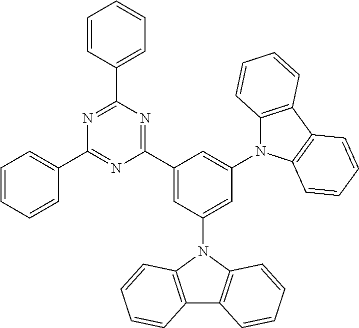 [SMILES: c1ccc(-c2nc(-c3ccccc3)nc(-c3cc(-n4c5ccccc5c5ccccc54)cc(-n4c5ccccc5c5ccccc54)c3)n2)cc1]

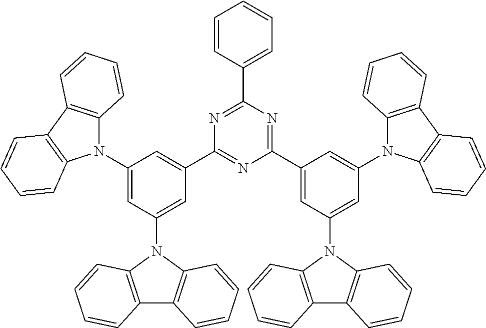 [SMILES: c1ccc(-c2nc(-c3cc(-n4c5ccccc5c5ccccc54)cc(-n4c5ccccc5c5ccccc54)c3)nc(-c3cc(-n4c5ccccc5c5ccccc54)cc(-n4c5ccccc5c5ccccc54)c3)n2)cc1]

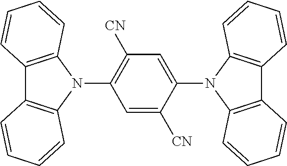 [SMILES: N#Cc1cc(-n2c3ccccc3c3ccccc32)c(C#N)cc1-n1c2ccccc2c2ccccc21]

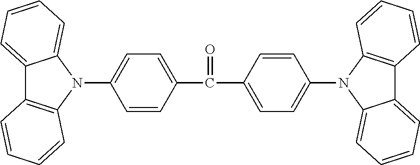 [SMILES: O=C(c1ccc(-n2c3ccccc3c3ccccc32)cc1)c1ccc(-n2c3ccccc3c3ccccc32)cc1]

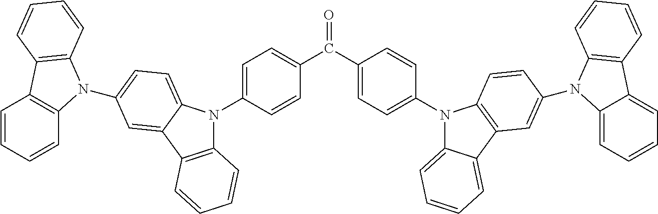 [SMILES: O=C(c1ccc(-n2c3ccccc3c3cc(-n4c5ccccc5c5ccccc54)ccc32)cc1)c1ccc(-n2c3ccccc3c3cc(-n4c5ccccc5c5ccccc54)ccc32)cc1]

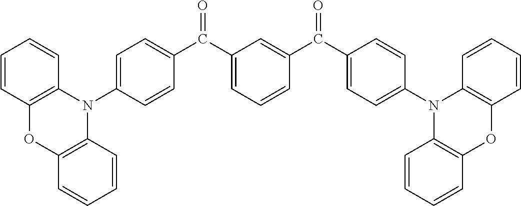 [SMILES: O=C(c1ccc(N2c3ccccc3Oc3ccccc32)cc1)c1cccc(C(=O)c2ccc(N3c4ccccc4Oc4ccccc43)cc2)c1]

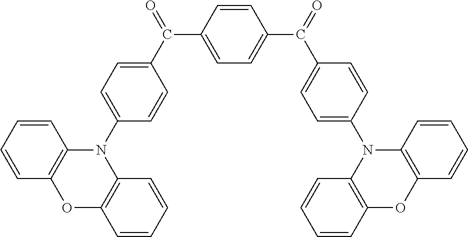 [SMILES: O=C(c1ccc(C(=O)c2ccc(N3c4ccccc4Oc4ccccc43)cc2)cc1)c1ccc(N2c3ccccc3Oc3ccccc32)cc1]

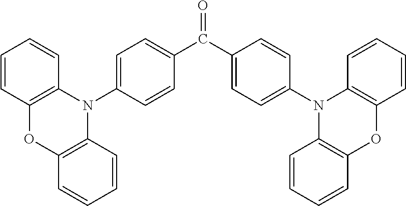 [SMILES: O=C(c1ccc(N2c3ccccc3Oc3ccccc32)cc1)c1ccc(N2c3ccccc3Oc3ccccc32)cc1]

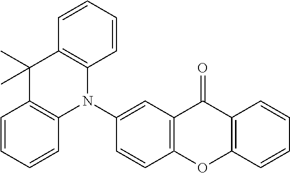 [SMILES: CC1(C)c2ccccc2N(c2ccc3oc4ccccc4c(=O)c3c2)c2ccccc21]

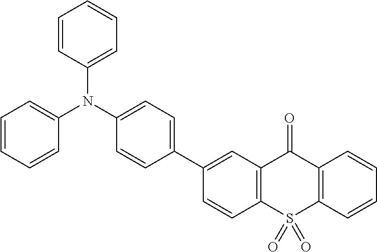 [SMILES: O=C1c2ccccc2S(=O)(=O)c2ccc(-c3ccc(N(c4ccccc4)c4ccccc4)cc3)cc21]

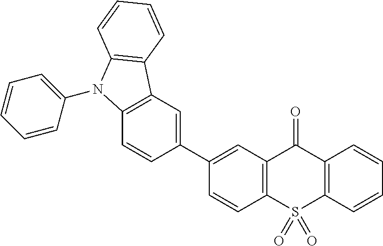 [SMILES: O=C1c2ccccc2S(=O)(=O)c2ccc(-c3ccc4c(c3)c3ccccc3n4-c3ccccc3)cc21]

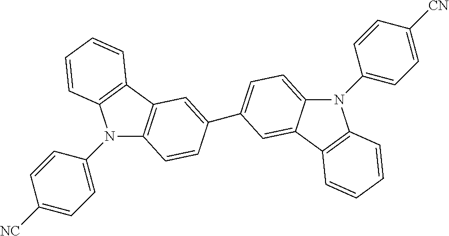 [SMILES: N#Cc1ccc(-n2c3ccccc3c3cc(-c4ccc5c(c4)c4ccccc4n5-c4ccc(C#N)cc4)ccc32)cc1]

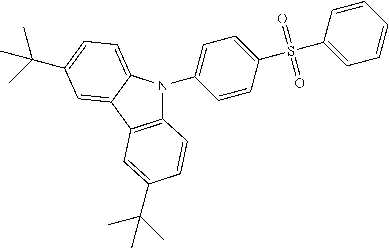 [SMILES: CC(C)(C)c1ccc2c(c1)c1cc(C(C)(C)C)ccc1n2-c1ccc(S(=O)(=O)c2ccccc2)cc1]

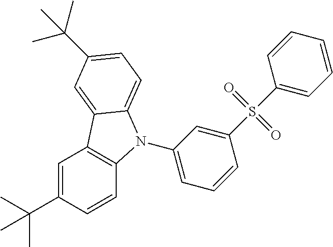 [SMILES: CC(C)(C)c1ccc2c(c1)c1cc(C(C)(C)C)ccc1n2-c1cccc(S(=O)(=O)c2ccccc2)c1]

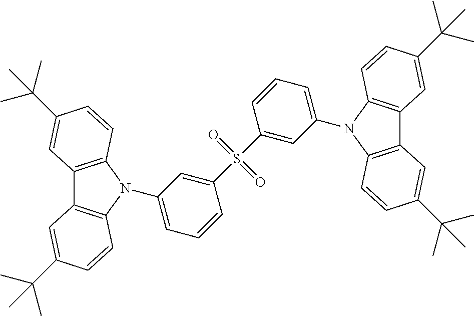 [SMILES: CC(C)(C)c1ccc2c(c1)c1cc(C(C)(C)C)ccc1n2-c1cccc(S(=O)(=O)c2cccc(-n3c4ccc(C(C)(C)C)cc4c4cc(C(C)(C)C)ccc43)c2)c1]

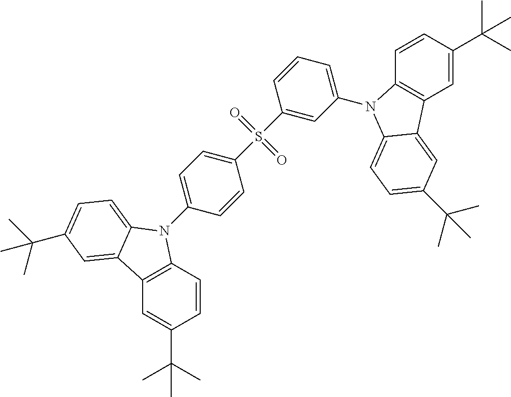 [SMILES: CC(C)(C)c1ccc2c(c1)c1cc(C(C)(C)C)ccc1n2-c1ccc(S(=O)(=O)c2cccc(-n3c4ccc(C(C)(C)C)cc4c4cc(C(C)(C)C)ccc43)c2)cc1]

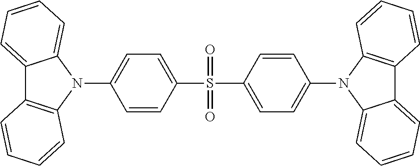 [SMILES: O=S(=O)(c1ccc(-n2c3ccccc3c3ccccc32)cc1)c1ccc(-n2c3ccccc3c3ccccc32)cc1]

Another object of the present disclosure is to provide a technical solution for OLED printing.

In one embodiment, the molecular weight of the thermally activated delayed fluorescence material is larger than or equal to 700 g/mol. In another embodiment, the molecular weight of the thermally activated delayed fluorescence material is larger than or equal to 900 g/mol. In another embodiment, the molecular weight of the thermally activated delayed fluorescence material is larger than or equal to 900 g/mol. In another embodiment, the molecular weight of the thermally activated delayed fluorescence material is larger than or equal to 1000 g/mol. In another embodiment, the molecular weight of the thermally activated delayed fluorescence material is larger than or equal to 1100 g/mol.

In one embodiment, the solubility of the thermally activated delayed fluorescence material in methylbenzene at 25° C. is larger than or equal to 10 mg/mL. In another embodiment, the solubility of the thermally activated delayed fluorescence material in methylbenzene at 25° C. is larger than or equal to 15 mg/mL. In another embodiments, the solubility of the thermally activated delayed fluorescence material in methylbenzene at 25° C. is larger than or equal to 20 mg/mL.

The present disclosure further relates to a formulation or an ink including the thermally activated delayed fluorescence material described above or the polymer described above, and an organic solvent.

The viscosity and the surface tension of the ink are important parameters in the printing process. Appropriate surface tension parameter of the ink is suitable for the specific substrate and the specific printing method.

In one embodiment, the surface tension of the ink at the working temperature or at 25° C. is in a range from about 19 dyne/cm to 50 dyne/cm. In anther embodiment, the surface tension of the ink at the working temperature or at 25° C. is in a range from 22 dyne/cm to 35 dyne/cm. In another embodiment, the surface tension of the ink at the working temperature or at 25° C. is in a range from 25 dyne/cm to 33 dyne/cm.

In one embodiment, the viscosity of the ink at the working temperature or at 25° C. is in a range from about 1 cps to 100 cps. In another embodiment, the viscosity of the ink at the working temperature or at 25° C. is in a range from 1 cps to 50 cps. In another embodiment, the viscosity of the ink at the working temperature or at 25° C. is in a range from 1.5 cps to 20 cps. In another embodiment, the viscosity of the ink at the working temperature or at 25° C. is in a range from 4.0 cps to 20 cps.

The formulation so prepared is suitable for inkjet printing.

The viscosity can be adjusted by various methods, such as by selecting an appropriate solvent and the concentration of the functional material in the ink. According to the ink including the thermally activated delayed fluorescence material described above and the polymer described above, the ink may be conveniently adjusted in the appropriate range according to the used printing method.

In general, the weight ratio of the functional material included in the formulation is in a range from 0.3 wt % to 30 wt %. In one embodiment, the weight ratio of the functional material included in the formulation is in a range from 0.5 wt % to 20 wt %. In another embodiment, the weight ratio of the functional material included in the formulation is in a range from 0.5 wt % to 15 wt %. In another embodiment, the weight ratio of the functional material included in the formulation is in a range from 0.5 wt % to 10 wt %. In another embodiment, the weight ratio of the functional material included in the formulation is in a range from 1 wt % to 5 wt %.

In one embodiment, the organic solvent in the ink is selected from solvents based on aromatics or heteroaromatics, such as aliphatic chain/ring substituted aromatic solvents, or aromatic ketone solvents, or aromatic ether solvents.

In one embodiment, the organic solvent is selected from: the solvents based on aromatics or heteroaromatics, such as p-diisopropylbenzene, pentylbenzene, tetrahydronaphthalene, cyclohexyl benzene, chloronaphthalene, 1,4-dimethylnaphthalene, 3-isopropylbiphenyl, p-cymene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, 1-methoxynaphthalene, cyclohexylbenzene, dimethylnaphthalene, 3-isopropylbiphenyl, p-cymene, 1-methylnaphthalene, 1,2,4-trichlorobenzene, 1,3-dipropoxybenzene, 4,4-difluorodiphenylmethane, 1,2-dimethoxy-4-(1-propenyl)benzene, diphenylmethane, 2-phenylpyridine, 3-phenylpyridine, N-methyldiphenylamine 4-isopropylbiphenyl, α,α-dichlorodiphenylmethane, 4-(3-phenylpropyl)pyridine, benzylbenzoate, 1,1-di(3,4-dimethylphenyl)ethane, 2-isopropylnaphthalene, dibenzylether, and etc.; solvents based on ketones, such as 1-tetralone, 2-tetralone, 2-(phenylepoxy)tetralone, 6-(methoxyl)tetralone, acetophenone, phenylacetone, benzophenone, and derivatives thereof, such as 4-methylacetophenone, 3-methylacetophenone, 2-methylacetophenone, 4-methylphenylacetone, 3-methylphenylacetone, 2-methylphenylacetone, isophorone, 2,6,8-trimethyl-4-nonanone, fenchone, 2-nonanone, 3-nonanone, 5-nonanone, 2-demayone, 2,5-hexanedione, phorone, di-n-amyl ketone; aromatic ether solvents, such as 3-phenoxytoluene, butoxybenzene, benzylbutylbenzene, p-anisaldehyde dimethyl acetal, tetrahydro-2-phenoxy-2H-pyran, 1,2-dimethoxy 4-(1-propenyl)benzene, 1,4-benzodioxane, 1,3-dipropylbenzene, 2,5-dimethoxytoluene, 4-ethylphenetole, 1,2,4-trimethoxybenzene, 4-(1-propenyl)-1,2-dimethoxybenzene, 1,3-dimethoxybenzene, glycidyl phenyl ether, dibenzyl ether, 4-tert-butylanisole, trans-p-propenylanisole, 1,2-dimethoxybenzene, 1-methoxynaphthalene, diphenyl ether, 2-phenoxymethyl ether, 2-phenoxytetrahydrofuran, ethyl-2-naphthyl ether, pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether; and ester solvents, such as alkyl octoate, alkyl sebacate, alkyl stearate, alkyl benzoate, alkyl phenylacetate, alkyl cinnamate, alkyl oxalate, alkyl maleate, alkyl lactone, alkyl oleate, and etc.

In one embodiment, the organic solvent is selected from aliphatic ketones, such as 2-nonanone, 3-nonanone, 5-nonanone, 2-demayone, 2,5-hexanedione, 2,6,8-trimethyl-4-nonanone, phorone, di-n-pentyl ketone, and etc.; or aliphatic ethers, such as amyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethyl ether alcohol ethyl methyl ether, triethylene glycol butyl methyl ether, tripropylene glycol dimethyl ether, tetraethylene glycol dimethyl ether, and etc.

In one embodiment, the printing ink further includes another organic solvent. Examples of the another organic solvent include methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxy toluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, decalin, indene, and/or mixtures thereof.

In one embodiment, the formulation is a solution.

In another embodiment, the formulation is a suspension liquid.

In one embodiment, the formulation includes 0.01 wt % to 20 wt % of the thermally activated delayed fluorescence material (or mixtures thereof). In another embodiment, the formulation includes 0.1 wt % to 15 wt % of the thermally activated delayed fluorescence material (or mixtures thereof). In another embodiment, the formulation includes 0.2 wt % to 10 wt % of the thermally activated delayed fluorescence material (or mixtures thereof). In another embodiment, the formulation includes 0.25 wt % to 5 wt % of the thermally activated delayed fluorescence material (or mixtures thereof).

The present disclosure further relates to the application of the formulation as the paint or the printing ink to make an organic electronic device, such as by a printing method or a coating method.

The appropriate printing technology or coating technology includes, but is not limited to inkjet printing, nozzle printing, typographic printing, screen printing, dip coating, spin coating, blade coating, roller printing, torsion roller printing, lithographic printing, flexographic printing, rotary printing, spray coating, brush coating, pad printing, slot die coating, and etc. The first preference is inkjet printing, nozzle printing, and typographic printing. The solution or the suspension liquid may further includes one or more components, such as a surfactant compound, a lubricant, a wetting agent, a dispersant, a hydrophobic agent, a binder, to adjust the viscosity and the film forming property and to improve the adhesion property. The detailed information relevant to the printing technology and requirements of the printing technology to the solution, such as solvent, concentration, and viscosity, may be referred to Handbook of Print Media: Technologies and Production Methods, Helmut Kipphan, ISBN 3-540-67326-1.

According to the thermally activated delayed fluorescence material described above, the present disclosure further provides an application of the thermally activated delayed fluorescence material described above or the polymer described above in the organic electronic device.

The organic electronic device may be selected from organic light emitting diode (OLED), organic photovoltaic cell (OPV), organic light emitting electrochemical cell (OLEEC), organic field effect transistor (OFET), organic light emitting field effect transistor, organic laser, organic spin electron device, organic sensor, organic plasmon emitting diode, and etc.

In one embodiment, the organic electronic device is OLED, OLEEC, or organic light emitting field effect transistor.

In one embodiment, the thermally activated delayed fluorescence material is applied in a light emitting layer of the organic electronic device.

The present disclosure further relates to an organic electronic device including the thermally activated delayed fluorescence material described above or the polymer described above.

In general, the organic electronic device at least includes a cathode, an anode, and a functional layer located therebetween. The functional layer includes the thermally activated delayed fluorescence material described above or the polymer described above.

The organic electronic device may be selected from organic light emitting diode (OLED), organic photovoltaic cell (OPV), organic light emitting electrochemical cell (OLEEC), organic field effect transistor (OFET), organic light emitting field effect transistor, organic laser, organic spin electron device, organic sensor, organic plasmon emitting diode, and etc.

In one embodiment, the organic electronic device is an organic electroluminescent device, such as OLED, OLEEC, or organic light emitting field effect transistor.

In some embodiments, the light emitting layer of the organic electroluminescent device includes the thermally activated delayed fluorescence material described above or the polymer described above; or includes the thermally activated delayed fluorescence or the polymer described above, and the phosphorescent emitter; or includes the thermally activated delayed fluorescence material described above or the polymer described above, and the host material; or includes the thermally activated delayed fluorescence material described above or the polymer described above, the phosphorescent emitter, and the host material.

The organic electroluminescent device, such as OLED, includes a substrate, an anode, a light emitting layer, and a cathode.

The substrate can be opaque or transparent. The transparent substrate can be used to make a transparent luminescent device, which can be referred to, for example, Bulovic et al., Nature, 1996, 380, page 29 and Gu et al., Appl. Phys. Lett., 1996, 68, page 2606. The substrate can be rigid or flexible. The substrate can be plastic, metal, a semiconductor wafer, or glass. In one embodiment, the substrate has a smooth surface. The substrate without any surface defects is the particular ideal selection. In one embodiment, the substrate is flexible and can be selected from a polymer thin film or a plastic which have the glass transition temperature Tg larger than 150° C. in one embodiment, larger than 200° C. in another embodiment, larger than 250° C. in another embodiment, and larger than 300° C. in another embodiment. Suitable examples of the flexible substrate include polyethylene terephthalate (PET) and polyethylene 2,6-naphthalate (PEN).

The anode may include a conductive metal, a metallic oxide, or a conductive polymer. The anode can inject holes easily into the hole injection layer (HIL), the hole transport layer (HTL), or the light emitting layer. In one embodiment, the absolute value of the difference between the work function of the anode and the HOMO energy level or the valence band energy level of the p type semiconductor material as the HIL or the HTL is smaller than 0.5 eV in one embodiment, is smaller than 0.3 eV in another embodiment, and is smaller than 0.2 eV in another embodiment. Examples of the anode material include, but are not limited to Al, Cu, Au, Ag, Mg, Fe, Co, Ni, Mn, Pd, Pt, ITO, aluminum-doped zinc oxide (AZO), and etc. Other suitable anode materials are known and can be easily selected by one of ordinary skilled in the art. The anode material can be deposited by any suitable technologies, such as the suitable physical vapor deposition method which includes a radio frequency magnetron sputtering, a vacuum thermal evaporation, an electron beam, and etc. In some embodiments, the anode is patterned and structured. A patterned ITO conductive substrate may be purchased from market to prepare the device according to the present disclosure.

The cathode can include a conductive metal or metal oxide. The cathode can inject electrons easily into the electron injection layer (EIL) or the electron transport layer (ETL), or directly injected into the light emitting layer. In one embodiment, the absolute value of the difference between the work function of the cathode and the LUMO energy level or the valence band energy level of the n type semiconductor material as the EIL or the ETL is smaller than 0.5 eV, such as smaller than 0.3 eV, and smaller than 0.2 eV. In principle, all materials capable of using as the cathode of the OLED can be used as the cathode material of the device of the present disclosure. Examples of the cathode material include, but are not limited to, Al, Au, Ag, Ca, Ba, Mg, LiF/Al, Mg—Ag alloy, BaF₂/Al, Cu, Fe, Co, Ni, Mn, Pd, Pt, ITO, and etc. The cathode material may be deposited by any suitable technologies, such as the suitable physical vapor deposition method which includes a radio frequency magnetron sputtering, a vacuum thermal evaporation, an electron beam, and etc.

The OLED can further include other functional layers, such as a hole injection layer (HIL), a hole transport layer (HTL), an electron blocking layer (EBL), an electron injection layer (EIL), an electron transport layer (ETL), and a hole blocking layer (HBL). Materials suitable for these functional layers are described in detail in the above and in WO2010135519A1, US20090134784A1, and WO2011110277A1, all contents of which are specially incorporated herein by reference.

In one embodiment, the light emitting layer of the organic electroluminescent device is prepared by the same way as the formulation described above.

In one embodiment, the emission wavelength of the organic electroluminescent device is in a range from 300 nm to 1000 nm. In another embodiment, the emission wavelength of the organic electroluminescent device is in a range from 350 nm to 900 nm. In another embodiment, the emission wavelength of the organic electroluminescent device is in a range from about 400 nm to about 800 nm.

The present disclosure further relates to the application of the organic electronic device in various electronic apparatuses, such as display apparatus, lighting apparatus, light source, sensor, etc.

The present disclosure further relates to electronic apparatuses, such as display apparatus, lighting apparatus, light source, sensor, and the like, including the organic electronic device described above.

The disclosure will now be described with reference to the preferred embodiments, but the disclosure is not to be construed as being limited to the following examples. It is to be understood that the appended claims are intended to cover the scope of the disclosure. Those skilled in the art will understand that modifications may be made to various embodiments of the disclosure with the teaching of the present disclosure, which will be covered by the spirit and scope of the claims of the disclosure.

### Example 1

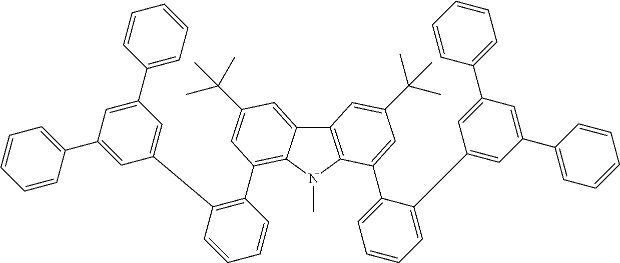 [SMILES: Cn1c2c(-c3ccccc3-c3cc(-c4ccccc4)cc(-c4ccccc4)c3)cc(C(C)(C)C)cc2c2cc(C(C)(C)C)cc(-c3ccccc3-c3cc(-c4ccccc4)cc(-c4ccccc4)c3)c21]

### Preparation of 1,8-bis(2-(3 phenylphenyl))phenyl-3,6-di-tert-butyl-9-methyl carbazole

4.5 g (10 mmol) of 1,8-dibromo-3,6-di-tert-butyl-9-methyl carbazole, 7.7 g (22 mmol) of 2-(3′,5′-diphenylphenyl)phenylboronic acid, 6.9 g (50 mmol) of potassium carbonate, 1.15 g (1 mmol) of Pd(PPh₃)₄, 100 mL of methylbenzene, 25 mL of water, and 25 mL of ethanol are added into a 250 mL three-necked flask and reacted at 110° C. in N₂ atmosphere, during which TLC is used to monitor the reaction process. After the reaction, the reacted liquid is cooled to room temperature, poured into water to wash away K₂CO₃, and suction filtrated to obtain a solid product. The solid product is washed by dichloromethane and recrystallized by ethanol to obtain 7.2 g of white solid powder product, i.e., 1,8-bis(2-(3′,5′-diphenylphenyl))phenyl-3,6-di-tert-butyl-9-methyl carbazole. MS(ASAP)=902.4.

### Example 2

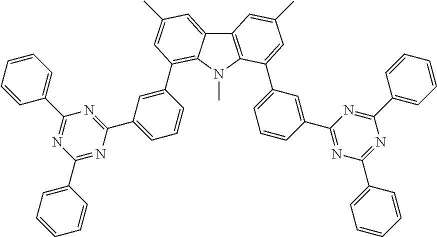 [SMILES: Cc1cc(-c2cccc(-c3nc(-c4ccccc4)nc(-c4ccccc4)n3)c2)c2c(c1)c1cc(C)cc(-c3cccc(-c4nc(-c5ccccc5)nc(-c5ccccc5)n4)c3)c1n2C]

### Preparation of 1,8-bis(3-(4′ 6′-diphenyl-1′,3′,5′-triazinyl))phenyl-3,6-dimethyl-9-methyl carbazole

3.65 g (10 mmol) of 1,8-dibromo-3,6-di-tert-butyl-9-methyl carbazole, 7.73 g (22 mmmol) of 3-(4′,6′-diphenyl-1′,3′,5′-triazinyl)phenylboronic acid, 6.9 g (50 mmol) of potassium carbonate, 1.15 g (1 mmol) of Pd(PPh₃)₄, 100 mL of methylbenzene, 25 mL of water, and 25 mL of ethanol are added into a 250 mL three-necked flask and reacted at 110° C. in N₂ atmosphere, during which TLC is used to monitor the reaction process. After the reaction, the reacted liquid is cooled to room temperature, poured into water to wash away K₂CO₃, and suction filtrated to obtain a solid product. The solid product is washed by dichloromethane and recrystallized by methylbenzene/methanol mixture solvent to obtain white solid powder product, i.e., 1,8-bis(3-(4′,6′-diphenyl-1′,3′,5′-triazinyl))phenyl-3,6-dimethyl-9-methyl carbazole. MS(ASAP)=908.2.

### Example 3

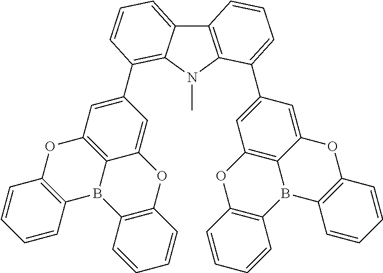 [SMILES: Cn1c2c(-c3cc4c5c(c3)Oc3ccccc3B5c3ccccc3O4)cccc2c2cccc(-c3cc4c5c(c3)Oc3ccccc3B5c3ccccc3O4)c21]

### Preparation of 1,8-bis(4-(diphenylboryl-3′,5′-dioxy))phenyl-9-methyl carbazole

3.38 g (10 mmol) of 1,8-dibromo-3,6-dimethyl-9-methyl carbazole, 6.82 g (22 mmmol) of 4-(diphenylboryl-3′,5′-dioxy)phenylboronic acid, 6.9 g (50 mmol) of potassium carbonate, 1.15 g (1 mmol) of Pd(PPh₃)₄, 100 mL of methylbenzene, 25 mL of water, and 25 mL of ethanol are added into a 250 mL three-necked flask and reacted at 110° C. in N₂ atmosphere, during which TLC is used to monitor the reaction process. After the reaction, the reacted liquid is cooled to room temperature, poured into water to wash away K₂CO₃, and suction filtrated to obtain a solid product. The solid product is washed by dichloromethane and recrystallized by methylbenzene/petroleum ether mixture solvent to obtain white solid powder product, i.e., 1,8-bis(4-(diphenylboryl-3′,5′-dioxy))phenyl-9-methyl carbazole. MS(ASAP)=718.4.

### Example 4

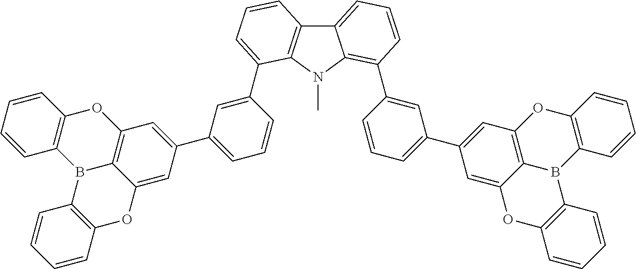 [SMILES: Cn1c2c(-c3cccc(-c4cc5c6c(c4)Oc4ccccc4B6c4ccccc4O5)c3)cccc2c2cccc(-c3cccc(-c4cc5c6c(c4)Oc4ccccc4B6c4ccccc4O5)c3)c21]

### Preparation of 1,8-bis(3-(4-(diphenylboryl-3′,5′-dioxy))phenyl)phenyl-9-methyl carbazole

1,8-bis(3-(4-(diphenylboryl-3′,5′-dioxy))phenyl)phenyl-9-methyl carbazole of Example 4 is prepared by the substantially same way as 1,8-bis(4-(diphenylboryl-3′,5′-dioxy))phenyl-9-methyl carbazole of Example 3, except that one intermediate is changed from 4-(diphenylboryl-3′,5′-dioxy)phenylboronic acid to (3-(4-(diphenylboryl-3′,5′-dioxy))phenyl)phenylboronic acid. The reaction temperatures and the reaction times in the reaction processes of are all the same. The final product 1,8-bis(3-(4-(diphenylboryl-3′,5′-dioxy))phenyl)phenyl-9-methyl carbazole is obtained by Suzuki reaction with the Pd(0) catalyst. MS(ASAP)=870.5.

### Example 5

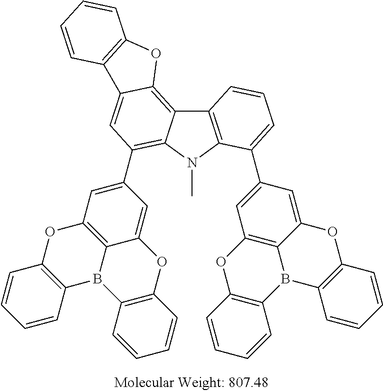 [SMILES: Cn1c2c(-c3cc4c5c(c3)Oc3ccccc3B5c3ccccc3O4)cccc2c2c3oc4ccccc4c3cc(-c3cc4c5c(c3)Oc3ccccc3B5c3ccccc3O4)c21]

### Preparation of 4,6-bis(5,9-dioxy-13b-borylnaphtho[3,2,1-de]heteroanthryl-7-yl)-5-methyl-5H-benzofuro[3,2-c] carbazole

4,6-bis(5,9-dioxy-13b-borylnaphtho[3,2,1-de]heteroanthryl-7-yl)-5-methyl-5H-benzofuro[3,2-c]carbazole of Example 5 is prepared by the substantially same way as 1,8-bis(4-(diphenylboryl-3′,5′-dioxy))phenyl-9-methyl carbazole of Example 3, except that one intermediate is changed from 1,8-dibromo-3,6-dimethyl-9-methyl carbazole to 4,6-dibromo-5-methyl-5H-benzofuro[3,2-c]carbazole. The reaction temperatures and the reaction times in the reaction processes are all the same. The final product 4,6-bis(5,9-dioxy-13b-borylnaphtho[3,2,1-de]heteroanthryl-7-yl)-5-methyl-5H-benzofuro[3,2-c]carbazole is obtained by Suzuki reaction with the Pd(0) catalyst. MS(ASAP)=807.24.

### Example 6

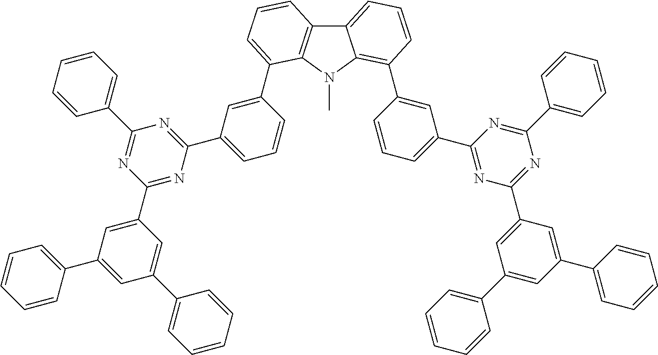 [SMILES: Cn1c2c(-c3cccc(-c4nc(-c5ccccc5)nc(-c5cc(-c6ccccc6)cc(-c6ccccc6)c5)n4)c3)cccc2c2cccc(-c3cccc(-c4nc(-c5ccccc5)nc(-c5cc(-c6ccccc6)cc(-c6ccccc6)c5)n4)c3)c21]

### Preparation of 1,8-bis(3-(6′-phenyl-4′-(3″,5″-diphenyl)phenyl-1′,3′,5′-triazinyl)) phenyl-9-methyl carbazole

1,8-bis(3-(6′-phenyl-4′-(3″,5″-diphenyl)phenyl-1′,3′,5′-triazinyl))phenyl-9-methyl carbazole of Example 6 is prepared by the substantially same way as 1,8-bis(3-(4′,6′-diphenyl-1′,3′,5′-triazinyl))phenyl-3,6-dimethyl-9-methyl carbazole of Example 2, except that one intermediate is changed from 3-(4′,6′-diphenyl-1′,3′,5′-triazinyl)phenylboronic acid to (3-(6′-phenyl-4′-(3″,5″-diphenyl)phenyl-1′,3′,5′-triazinyl)) phenylboronic acid. The reaction temperatures and the reaction times in the reaction processes are all the same. The final product 1,8-bis(3-(6′-phenyl-4′-(3″,5″-diphenyl)phenyl-1′,3′,5′-triazinyl))phenyl-9-methyl carbazole is obtained by Suzuki reaction with the Pd(0) catalyst. MS(ASAP)=1100.4.

The energy level of the organic compound material may be obtained by quantum computing, such as by Gaussian09W (Gaussian Inc.) using TD-DFT (Time-Dependent Density Functional Theory). The detailed simulation method may be referred to WO2011141110. First, the molecular geometry structure may be optimized by a semi-empirical method “Ground State/Semi-empirical/Default Spin/AM1” (Charge 0/Spin Singlet). Then the energy structure of the organic molecule is calculated by using the TD-DFT method for “TD-SCF/DFT/Default Spin/B3PW91” and the base group “6-31 G(d)” (Charge 0/Spin Singlet). The HOMO and LUMO energy levels are calculated according to the following calibration equations. S₁, T₁, and resonant factor f(S₁) are directly used.

HOMO (eV)=((HOMO(G)×27.212)−0.9899)/1.1206

LUMO (eV)=((LUMO(G)×27.212)−2.0041)/1.385

The HOMO(G) and LUMO(G) are direct computed results of Gaussian 09W, whose unit are Hartree. The results are listed in Table 1:

TABLE 1

| | HOMO | LUMO | f | T₁ | S₁ | |
|---|---|---|---|---|---|---|
| Product | [eV] | [eV] | (S₁) | [eV] | [eV] | ΔE_{ST} |
| Example 1 | −5.39 | −2.13 | 0.025 | 3.00 | 3.09 | 0.09 |
| Example 2 | −5.59 | −2.79 | 0.004 | 2.86 | 3.00 | 0.14 |
| Example 3 | −5.81 | −2.78 | 0.11 | 2.89 | 2.99 | 0.10 |
| Example 4 | −5.75 | −2.81 | 0.003 | 2.90 | 3.14 | 0.14 |
| Example 5 | −5.69 | −2.79 | 0.083 | 2.80 | 2.95 | 0.15 |
| Example 6 | −5.59 | −2.80 | 0.0024 | 2.95 | 3.00 | 0.05 |

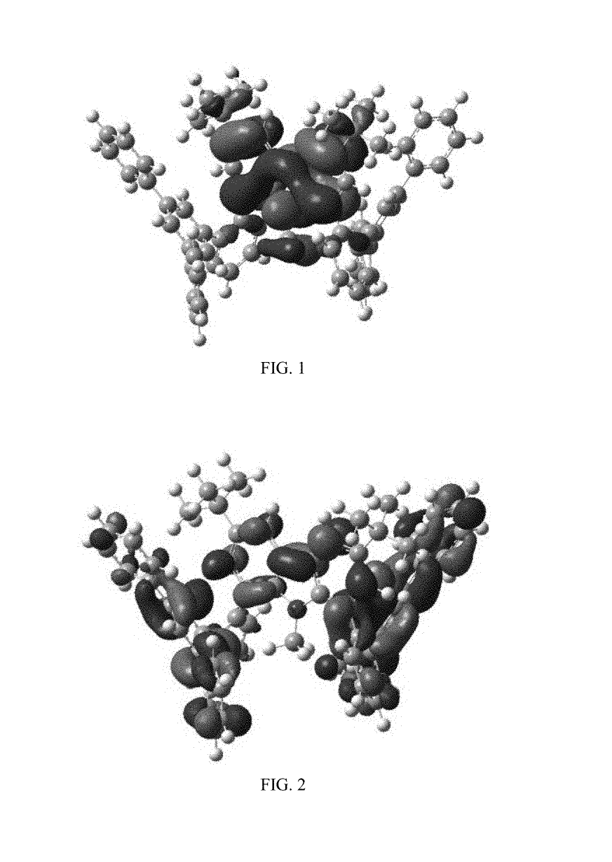

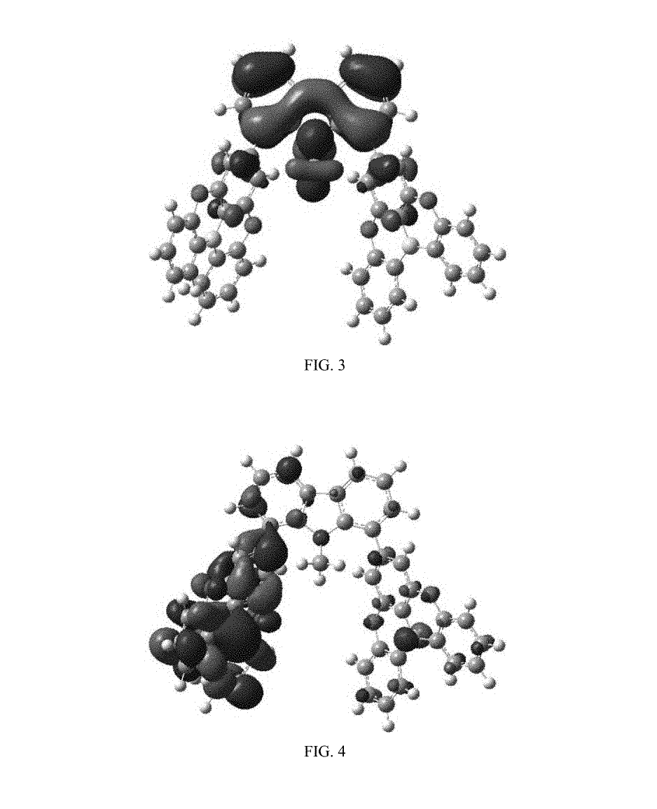

FIG. 1 to FIG. 4 show the HOMO and LUMO orbital distributions of the products obtained in Example 1 and Example 3.

It can be seen from Table 1 and FIG. 1 to FIG. 4 that the HOMO and LUMO orbital distributions of the product of Example 1 are preferably overlapped, so as to Example 3, so the resonant factors f(S₁) of the products of Example 1 and Example 3 are relatively high.

In the above Examples, the resonant factors f(S₁) are all ranged between 0.001 to 0.11, which can obviously improve the fluorescence quantum efficiencies of the materials. Meanwhile, the ΔE(S₁−T₁) values are not larger than 0.15 eV, which satisfy the condition of delayed fluorescence (ΔE(S₁−T₁)<0.30 eV).

A delayed fluorescent material labeled Ref 1 with D-A system structure is used to compare with the delayed fluorescent material described above:

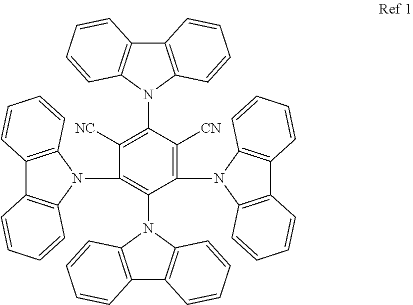 [SMILES: N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

Preparation of the OLED Devices:

OLED devices with structures of ITO/NPD(35 nm)/5%(1)˜(5):mCP(15 nm)/TPBi(65 nm)/LiF (1 nm)/A1 (150 nm)/cathode are prepared as follows:

a. cleaning the conductive glass substrate by various solvents such as chloroform, ketone, and isopropanol when first used, and then treating the conductive glass substrate with ultraviolet ozone plasma;

b. preparing HTL (35 nm), EML (15 nm), and ETL (65 nm) by thermal evaporation in a high vacuum (1×10⁻⁶ mbar);

c. preparing cathode LiF/A1 (1 nm/150 nm) by thermal evaporation in a high vacuum (1×10⁻⁶ mbar);

d. encapsulating the device with UV curable resin in a glove box filled with nitrogen gas.

The current-voltage (J-V) characteristics of the OLED devices are tested, and important parameters such as efficiency, lifetime, and external quantum efficiency are recorded. The results show that the luminous efficiency and the lifetime of the OLED 1 (corresponding to raw material (1)) are both more than three times that of the OLED Ref 1 (corresponding to raw material (Ref 1)), the luminous efficiency of the OLED 3 (corresponding to raw material (3)) is four times that of the OLED Ref 1 while the lifetime is more than six times, particularly the maximum external quantum efficiency of the OLED 3 is above 10%. It can be concluded that the OLED device prepared with the organic mixture of the present disclosure has significantly improved luminous efficiency and lifetime, and remarkably increased external quantum efficiency.

Although the above embodiments are described in detail, they are only a few embodiments of the present disclosure and may not be understood as limiting the scope of the present disclosure. It should be understood by those skilled in the art that various modifications and improvements may be made therein without departing from the theory of the present disclosure, which should also be seen in the scope of the present disclosure. The scope of the present disclosure should be defined by the appended claims.

1-22. (canceled)

23. A thermally activated delayed fluorescence material, comprising a structure unit represented by the following general formula (1): 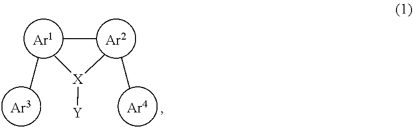
wherein Ar¹, Ar², Ar³, or Ar⁴ is selected from aromatic ring containing 3 to 20 carbon atoms, R₁ substituted aromatic ring containing 3 to 20 carbon atoms, heteroaromatic ring containing 2 to 20 carbon atoms, R₁ substituted heteroaromatic ring containing 2 to 20 carbon atoms, non-aromatic ring containing 1 to 20 carbon atoms, or R₁ substituted non-aromatic ring containing 1 to 20 carbon atoms;
X is a doubly bridging group or a triply bridging group linked to Ar¹, Ar², or Y by single bond or double bond;
Y is linked to X by single bond or double bond;
Y is selected from the group consisting of nothing, aromatic ring containing 3 to 20 carbon atoms, heteroaromatic ring containing 2 to 20 carbon atoms, non-aromatic ring containing 1 to 20 carbon atoms, B(OR₂)₂, Si(R₂)₃, alkyl ether group, alkyl thioether group containing 1 to 10 carbon atoms, alkyl, alkoxy, alkenyl, alkynyl, deuterides thereof, fluorides thereof, and R₁ substituted groups thereof, and a combination of two, three, or four thereof;
R₁ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, or cycloalkyl;
R₂ is selected from H, D, aliphatic alkyl containing 1 to 10 carbon atoms, aromatic hydrocarbon group containing 1 to 10 carbon atoms, aromatic ring containing 5 to 10 ring atoms, substituted aromatic ring containing 5 to 10 ring atoms, heteroaryl containing 5 to 10 ring atoms, or substituted heteroaryl containing 5 to 10 ring atoms.

24. The thermally activated delayed fluorescence material of claim 23, wherein an energy difference ΔE(S₁−T₁) between a singlet state and a triplet state of the thermally activated delayed fluorescence material is smaller than or equal to 0.30 eV.

25. The thermally activated delayed fluorescence material of claim 23, wherein X is selected from one of the following groups: 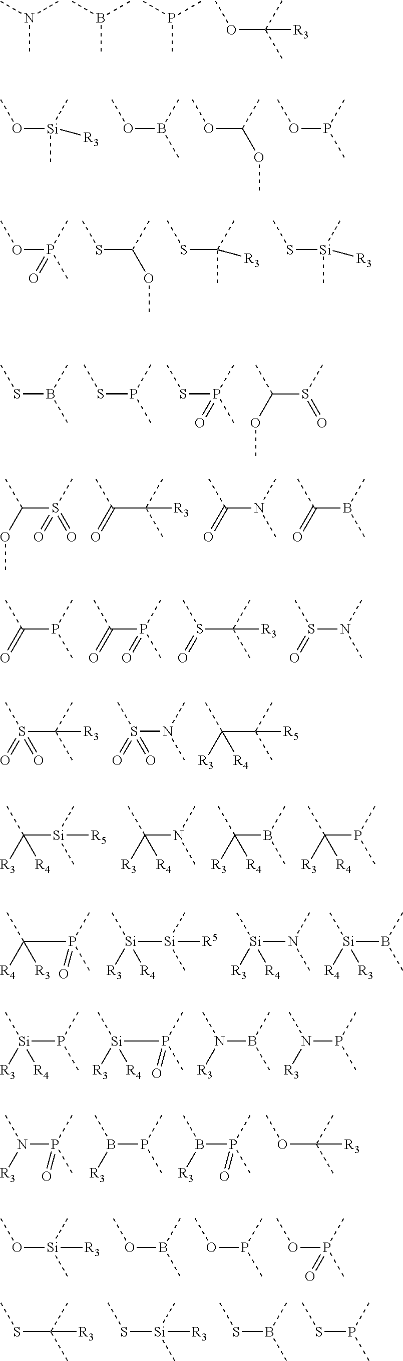 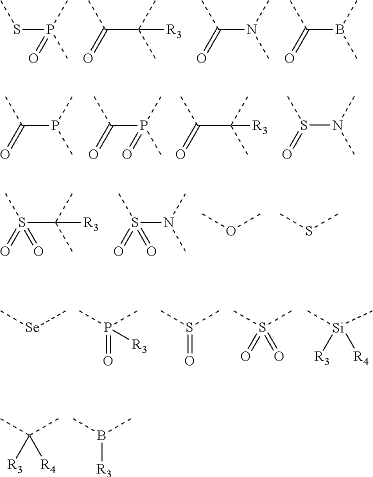
wherein R₃, R₄, or R₅ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, or cycloalkyl;
dashed lines in the above groups represent bonds linked to Ar¹, Ar², and Y.

26. The thermally activated delayed fluorescence material of claim 23, wherein Ar¹ or Ar² is selected from one of following groups: 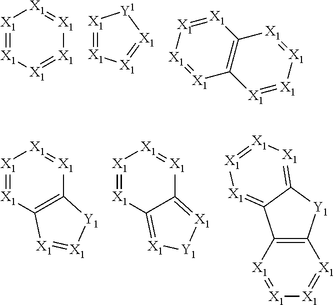
wherein X₁ is CR⁶ or N;
Y₁ is selected from CR⁷R⁸, SiR⁹R¹⁰, NR¹¹, C(═O), S, or O;
R₆, R₇, R₈, R₉, R₁₀, or R₁₁ is selected from H, D, straight chain alkyl containing 1 to 20 carbon atoms, alkoxy containing 1 to 20 carbon atoms, thioalkoxy containing 1 to 20 carbon atoms, branched chain alkyl containing 3 to 20 carbon atoms, cyclic alkyl containing 3 to 20 carbon atoms, silyl containing 3 to 20 carbon atoms, substituted ketone group containing 1 to 20 carbon atoms, alkoxycarbonyl containing 2 to 20 carbon atoms, aryloxycarbonyl containing 7 to 20 carbon atoms, cyano group, carbamoyl, haloformyl, formyl, isocyano group, isocyanate, thiocyanate, isothiocyanate, hydroxy, nitryl, CF₃, Cl, Br, F, crosslinkable group, aromatic ring containing 5 to 40 carbon atoms, substituted aromatic ring containing 5 to 40 carbon atoms, heteroaromatic ring containing 5 to 40 carbon atoms, substituted heteroaromatic ring containing 5 to 40 carbon atoms, aryloxy containing 5 to 40 carbon atoms, heteroaryloxy containing 5 to 40 carbon atoms, and combinations thereof.

27. The thermally activated delayed fluorescence material of claim 23, wherein Ar¹ or Ar² is selected from the following groups and the following groups substituted with R₁:

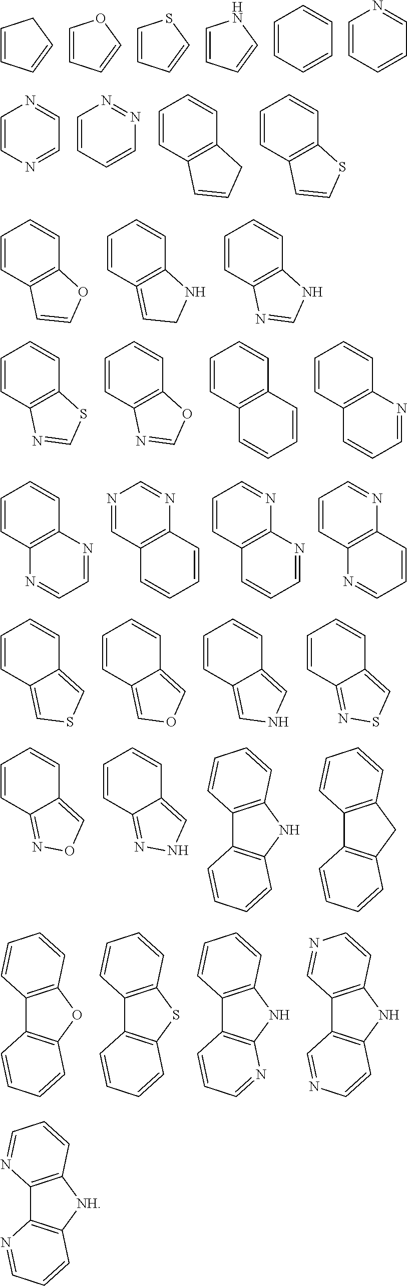

28. The thermally activated delayed fluorescence material of claim 23, wherein the thermally activated delayed fluorescence material is selected from one of compounds represented by the following general formulas: 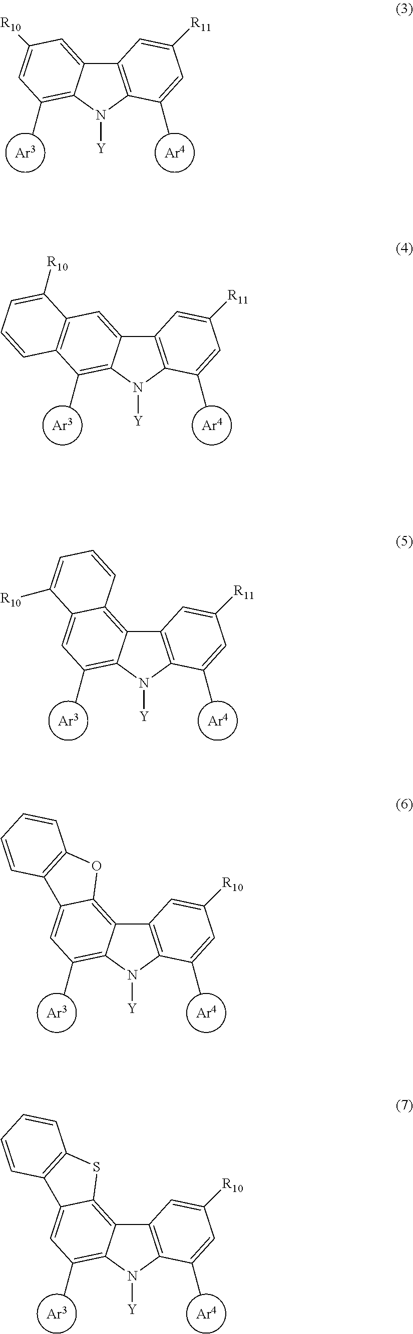 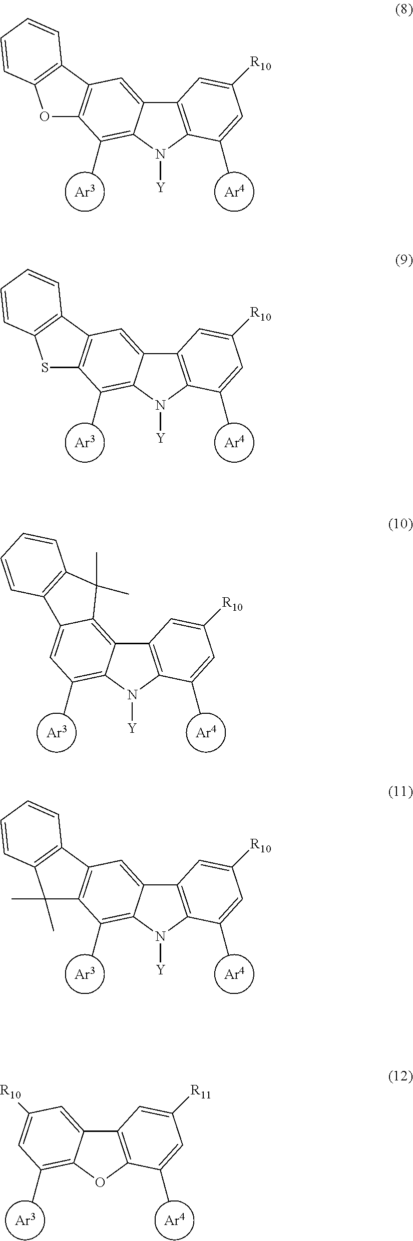 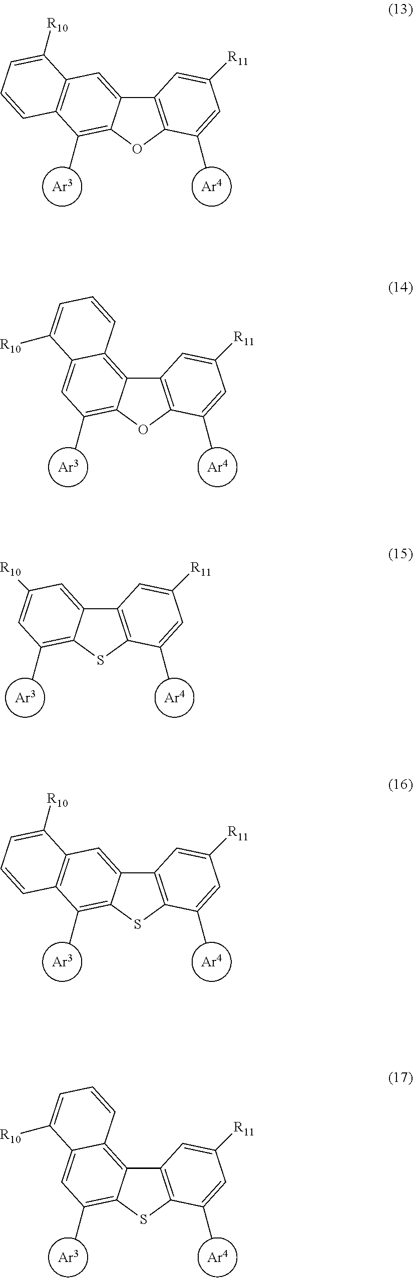 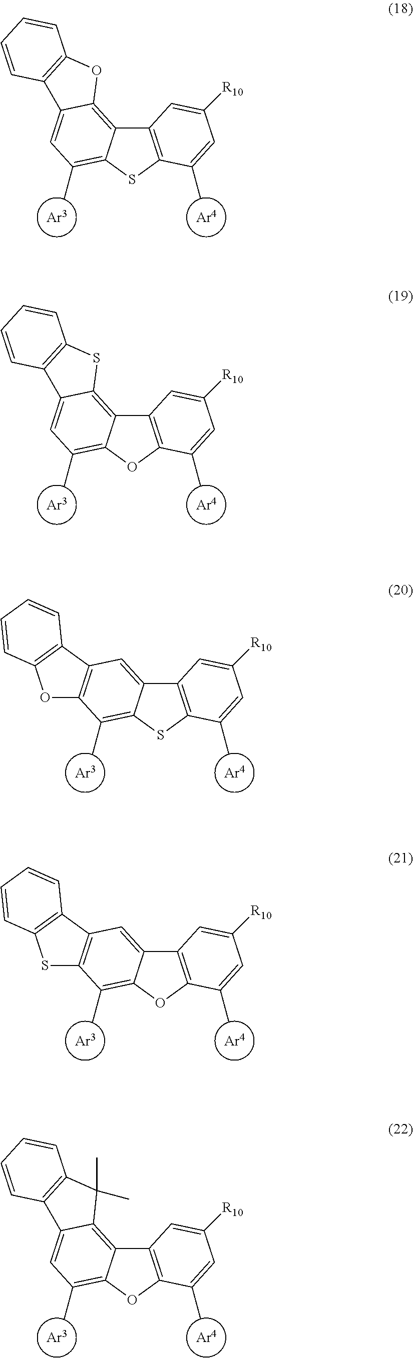 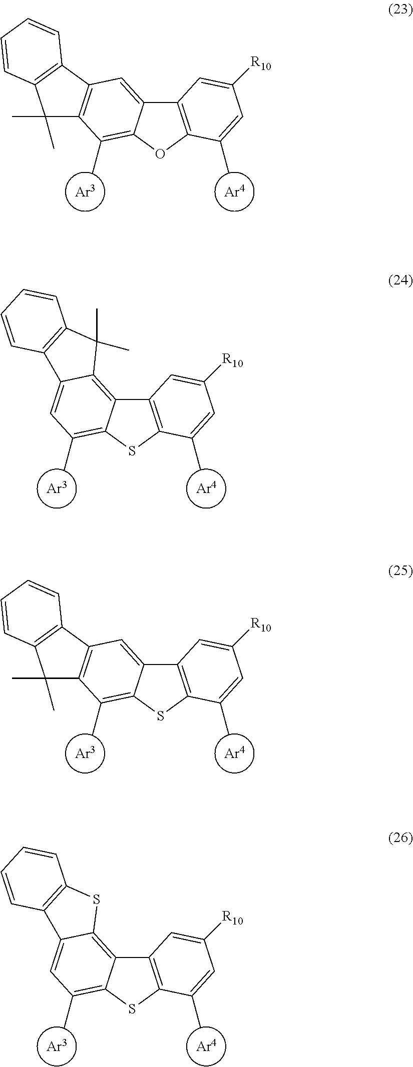
wherein R₁₀ or R₁₁ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, or cycloalkyl.

29. The thermally activated delayed fluorescence material of claim 23, wherein Ar³ or Ar⁴ comprises one or combinations of the following structure units or substituted following structure units: 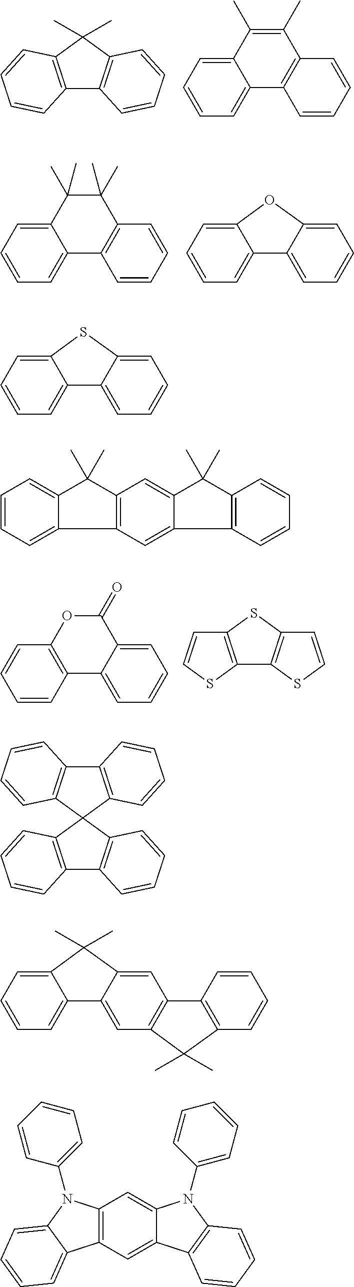 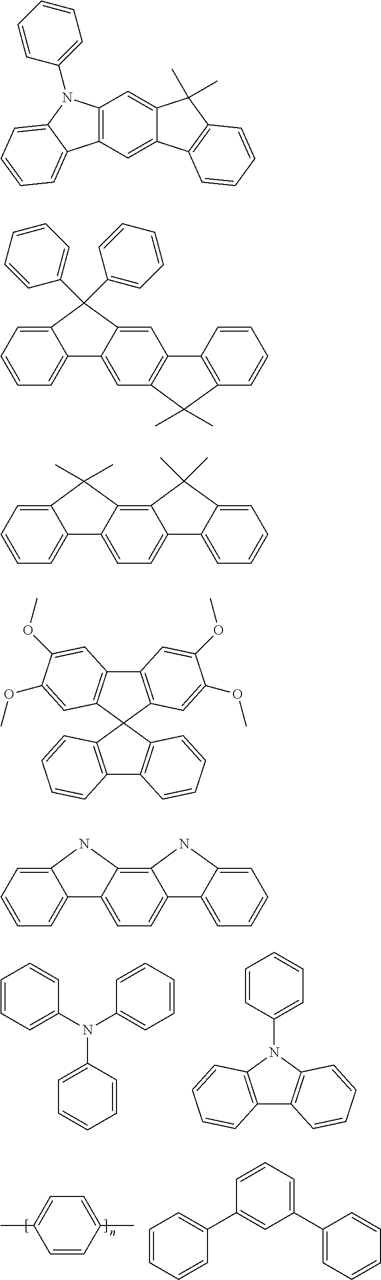 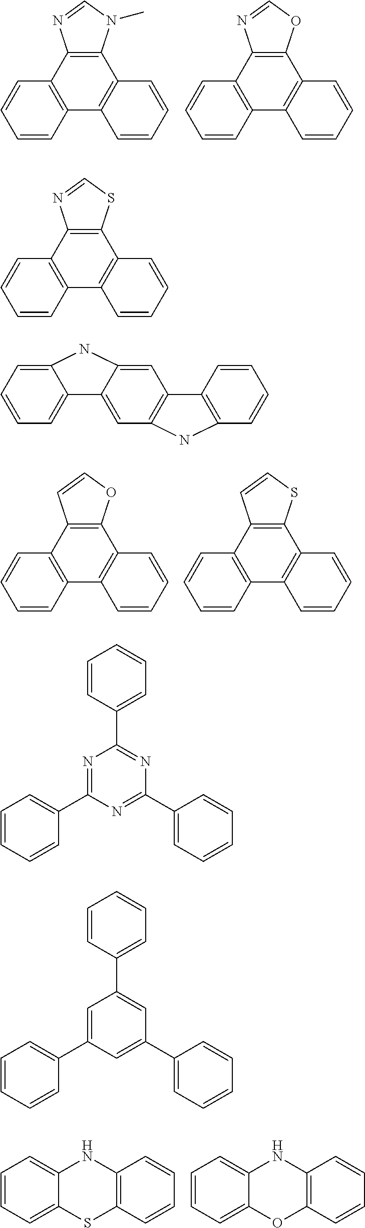
wherein n is 1, 2, 3, or 4.

30. The thermally activated delayed fluorescence material of claim 23, wherein at least one of Ar³ and Ar⁴ comprises at least one of a donor group and an acceptor group.

31. The thermally activated delayed fluorescence material of claim 30, wherein the donor group is selected from one of the following groups or substituted following groups:

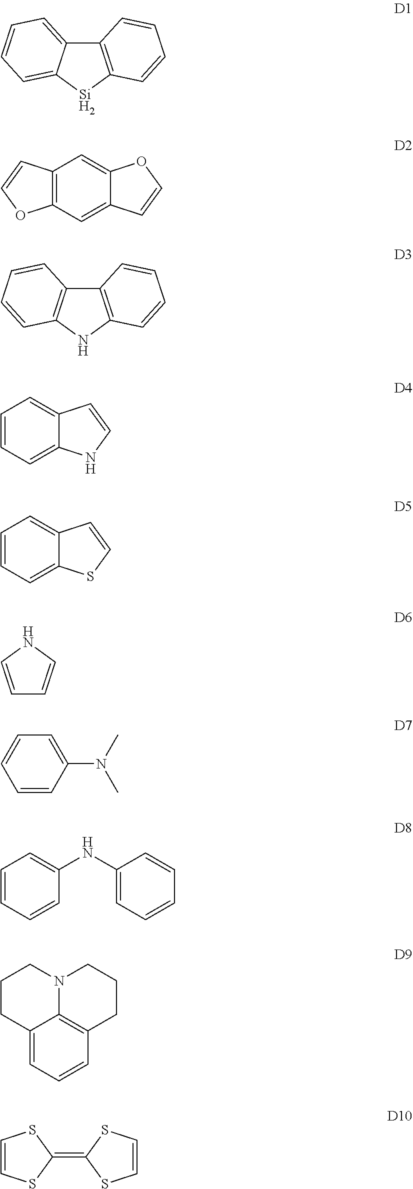

32. The thermally activated delayed fluorescence material of claim 30, wherein the acceptor group is selected from F, cyano group, and one of structure units comprising the following groups: 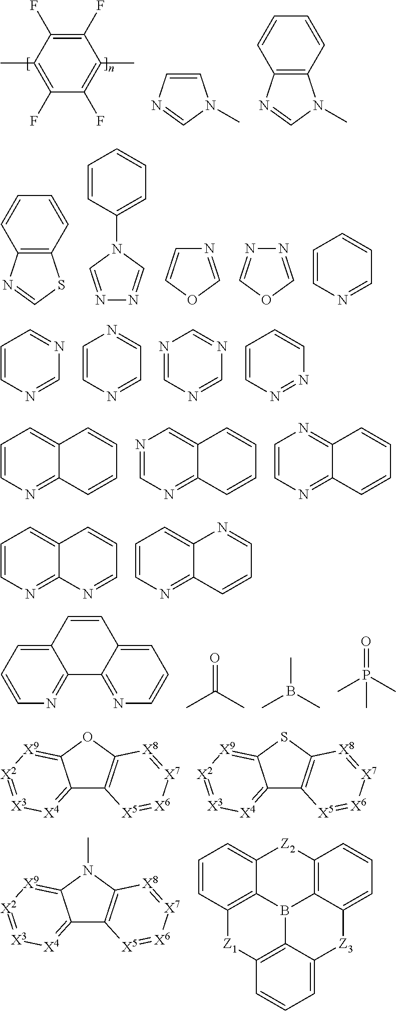
wherein n is 1, 2, 3, or 4;
X², X³, X⁴, X⁵, X⁶, X⁷, X⁸, or X⁹ is selected from CR or N, at least one of X², X³, X⁴, X⁵, X⁶, X⁷, X⁸, and X⁹ is N;
Z₁, Z₂, or Z₃ is N(R), C(R)₂, Si(R)₂, O, C═N(R), C═C(R)₂, P(R), P(═O)R, S, S═O, SO₂, or nothing, at least one of Z₁, Z₂, or Z₃ is not nothing;
R is selected from one of hydrogen, alkyl, alkoxy, amino, alkenyl, alkynyl, aralkyl, heteroaralkyl, aromatic ring, and heteroaromatic ring.

33. The thermally activated delayed fluorescence material of claim 23, wherein Y is selected from one of C1˜C10 alkyl, C1˜C10 alkoxy, C2˜C10 alkenyl, C2˜C10 alkynyl, C3˜C10 aromatic ring, C2˜C10 heteroaromatic ring, R₁ substituted C3˜C10 aromatic ring, R₁ substituted C2˜C10 heteroaromatic ring, deuterides thereof and fluorides thereof.

34. The thermally activated delayed fluorescence material of claim 33, wherein Y is selected from —CH₃, —CD₃, —CF₃, ethyl, n-propyl, isopropyl, cyclopropyl, n-butyl, isobutyl, sec-butyl, tert-butyl, cyclobutyl, 2-methylbutyl n-pentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, octynyl, methoxy, ethoxy, n-propoxy, isopropoxy, n-butoxy, isobutoxy, sec-butoxy, tert-butoxy, 2-methylbutoxy, benzene, naphthalene, pyrene, dihydropyrene, chrysene, perylene, naphthacene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthaimidazole, phenanthroimidazole, imidazopyridine, imidazopyrazine, imidazoquinoxaline, oxazole, benzoxazole, naphthoxazole, anthraxoxazole, phenanthroxazole, isoxazole, 1,2-thiazole, 1,3-thiazole, benzothiazole, pyridazine, benzopyridazine, pyrimidine, benzopyrimidine, quinoxaline, pyrazine, 1,5-naphthyridine, nitrocarbazole, benzoporphyrin, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole. 1,2,4,5-tetrazine, 1,2,3,4-tetrazine, 1,2,3,5-tetrazine, purine, pteridine, indolizine, or benzothiadiazole.

35. A polymer, wherein one repeating unit of the polymer comprises the thermally activated delayed fluorescence material of claim 23.

36. The polymer of claim 35, wherein the polymer is a non-conjugated polymer, the thermally activated delayed fluorescence material is located on side chains of the polymer.

37. The polymer of claim 35, wherein the polymer is a conjugated polymer.

38. An organic electronic device, comprising the thermally activated delayed fluorescence material of claim 23.

39. The organic electronic device of claim 38, wherein the organic electronic device is selected from one of an organic light emitting diode, an organic photovoltaic cell, an organic light emitting cell, an organic field effect transistor, an organic light emitting field effect transistor, an organic sensor, and an organic plasmon emitting diode.

40. The organic electronic device of claim 38, wherein the organic electronic device is an electroluminescent device;
a light emitting layer of the electroluminescent device comprises the thermally activated delayed fluorescence material comprising a structure unit represented by the following general formula (1): 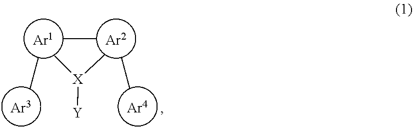
wherein Ar¹, Ar², Ar³, or Ar⁴ is selected from aromatic ring containing 3 to 20 carbon atoms, R₁ substituted aromatic ring containing 3 to 20 carbon atoms, heteroaromatic ring containing 2 to 20 carbon atoms, R₁ substituted heteroaromatic ring containing 2 to 20 carbon atoms, non-aromatic ring containing 1 to 20 carbon atoms, or R₁ substituted non-aromatic ring containing 1 to 20 carbon atoms;
X is a doubly bridging group or a triply bridging group linked to Ar¹, Ar², or Y by single bond or double bond;
Y is linked to X by single bond or double bond;
Y is selected from the group consisting of nothing, aromatic ring containing 3 to 20 carbon atoms, heteroaromatic ring containing 2 to 20 carbon atoms, non-aromatic ring containing 1 to 20 carbon atoms, B(OR₂)₂, Si(R₂)₃, alkyl ether group, alkyl thioether group containing 1 to 10 carbon atoms, alkyl, alkoxy, alkenyl, alkynyl, deuterides thereof, fluorides thereof, and R₁ substituted groups thereof, and a combination of two, three, or four thereof;
R₁ is selected from H, F, Cl, Br, I, D, CN, NO₂, CF₃, B(OR₂)₂, Si(R₂)₃, straight chain alkyl, alkyl ether group, alkyl thioether group containing 1 to 10 carbon atoms, branched chain alkyl, or cycloalkyl;
R₂ is selected from H, D, aliphatic alkyl containing 1 to 10 carbon atoms, aromatic hydrocarbon group containing 1 to 10 carbon atoms, aromatic ring containing 5 to 10 ring atoms, substituted aromatic ring containing 5 to 10 ring atoms, heteroaryl containing 5 to 10 ring atoms, or substituted heteroaryl containing 5 to 10 ring atoms.

41. The organic electronic device of claim 40, wherein the light emitting layer of the electroluminescent device further comprises one or two of a phosphorescent host or a host material.
